(19) United States
(12) Patent Application Publication
(10) Pub. No.: US 2022/0151962 A1
(43) Pub. Date: May 19, 2022

(54) NOVEL INHIBITORS OF HISTONE DEACETYLASE 10

(71) Applicant: Deutsches Krebsforschungszentrum, Heidelberg (DE)

(72) Inventors: Aubry Miller, Heidelberg (DE); Raphael Steimbach, Heidelberg (DE); Magalie Geraldy, Mairy sur Marne (FR)

(21) Appl. No.: 17/440,147

(22) PCT Filed: Mar. 20, 2020
(86) PCT No.: PCT/EP2020/057867

§ 371 Date: Sep. 16, 2021

(30) Foreign Application Priority Data
Mar. 22, 2019 (EP) 19164703.1
Mar. 22, 2019 (EP) 19164715.5

(51) Int. Cl. A61K 31/167; A61K 31/166; A61K 31/404; A61K 31/416; A61K 31/18; A61K 31/4184

(52) U.S. Cl. CPC A61K 31/167; A61K 31/166; A61K 31/4184; A61K 31/416; A61K 31/18; A61K 31/404

(57) ABSTRACT

The present invention relates to novel inhibitors of histone deacetylase 10 (HDAC10), novel pharmaceutical compositions comprising such inhibitors, and to novel methods of treating diseases, such as cancer, autoimmune disorders or neurodegeneration, using such novel inhibitors or methods of using such novel inhibitors in organ transplantation.

The present invention relates to novel inhibitors of histone deacetylase 10 (HDAC10), novel pharmaceutical compositions comprising such inhibitors, and to novel methods of treating diseases, such as cancer, autoimmune disorders or neurodegeneration, using such novel inhibitors or methods of using such novel inhibitors in organ transplantation.

### BACKGROUND

The discovery of histone deacetylase 1 (HDAC1) by Taunton and Schreiber in 1996¹ provided the long sought-after enzyme target for substances like trichostatin A (TSA (1), FIG. 1) and suberanilohydroxamic acid (SAHA (2)). At the time, 1 and 2 were reported to increase histone lysine acetylation levels, thereby inducing cellular differentiation, but their mechanism(s) of action were unknown.² Taunton and Schreiber's disclosure launched a now two-decade's long effort to discover inhibitors of HDACs, currently a family of 18 functionally related isozymes.^{3_4} During this time it has also become clear that HDACs have a broader role than catalyzing the hydrolysis of acetylated histone lysines: not only do they act in both the nucleus and the cytoplasm, but they catalyze the removal of acyl groups from a variety of different proteins.^{5_9} The 18 HDACs are grouped into four different classes based on homology to their yeast orthologs as follows: Class I (HDAC1, -2, -3, and -8); Class II, which is subdivided into Class IIA (HDAC4, -5, -7, and -9) and Class IIB (HDAC6 and -10); Class III (sirtuin1-7); and Class IV (HDAC11).¹⁰ While Classes I, IIA-B, and IV are Zn²⁺-dependent amidohydrolases, the Class III sirtuins are mechanistically distinct NAD+-dependent enzymes. For this reason, the sirtuins are often considered separately in discussions of “HDAC inhibitors”. Currently, there are four HDAC inhibitor drugs approved in the U.S. (2-5), one in China (6), many other candidates undergoing clinical trials, and dozens of reported inhibitors (FIG. 1). The approved drugs are used as anti-cancer agents, but HDAC inhibitors are also investigated in the treatment of autoimmune disorders, and neurodegeneration.³ Clinically used pan-HDAC inhibitor drugs (e.g. 2-5) can cause severe side effects, caused in part by their lack of selectivity. More isozyme-selective inhibitors are expected to overcome these liabilities and are likely to improve the clinical value of this target class.^{10_11} Moreover, the development of isozyme-selective chemical probes will be critical to further disentangle the biological role(s) of individual HDAC isozymes.¹² To date, the most significant focus and success in the development of selective inhibitors has been with the Class I enzymes, where selective inhibitors of HDAC1/2, HDAC3, and HDAC8 have been disclosed, and with the Class IIB enzyme HDAC6.³ Far fewer selective inhibitors of the Class IIA, HDAC10 or HDAC11 subtypes have been reported.^{13_17} In 2003, tubacin (7) was described as the first selective HDAC6 inhibitor (FIG. 1).¹⁸ In the intervening years, many additional HDAC6 inhibitors with good selectivity profiles have been described, the most well-known (besides tubacin) being tubastatin A (8).¹⁹ Indeed, both 7 and 8, along with ACY-738 (9)²⁰, are designated as HDAC6 chemical probes in the Chemical Probes Portal.²¹ Most HDAC6-selective inhibitors, like 8 and 9, achieve selectivity over Class I enzymes by incorporating a relatively bulky phenyl hydroxamate “linker” moiety in addition to a “cap group” that can make specific interactions with the HDAC6 protein surface (see 8, FIG. 1).

HDAC10 was first isolated in 2002 and annotated as a Class IIB HDAC based on its high similarity to HDAC6.^{22_24} Like HDAC6, HDAC10 appears to localize to both the nucleus and cytoplasm, and has been reported to interact with proteins having a variety of functions including transcription factors²⁵ and cyclins,²⁶ and to play a prominent role in homologous recombination²⁷ and Hsp-mediated VEGFR regulation.²⁸ While some clinical correlation studies have indicated an apparent tumor-suppressor function for HDAC10,^{29_33} a number of other studies highlight HDAC10 as a potential cancer drug target.^{34_37} In one study, high HDAC10 expression levels were found to correlate with poor clinical outcome for advanced stage 4 neuroblastoma patients who received chemotherapy.³⁷ Consistent with these findings, HDAC10 depletion in neuroblastoma cells interrupts autophagic flux and sensitizes cells for chemotherapy, and enforced HDAC10 expression protects neuroblastoma cells against doxorubicin treatment.³⁸

Additionally, it has been found that HDAC10 is involved in the regulation of PD-L1 Expression and immune tolerance mediated by antigen presenting cells (APCs).⁴² It could be shown that in macrophages isolated from HDAC10 knock-out mice exhibited an increased expression of MHC II molecules and a decreased expression of PD-L1. In an in vivo model, tumor growth was delayed in HDAC10 knock-out mice when compared to wild-type mice. Thus, the disruption of the HDAC10/PD-L1 axis could provide a novel target for cancer immunotherapy.

Recent investigations revealed HDAC10 involvement in the regulation of immune response by affecting Foxp3⁺ T-regulatory (Treg) cells. It was shown that HDAC10^{-/-} but not wild-type mice receiving fully MHC-mismatched cardiac transplants became tolerant and showed long-term allograft survival (>100 d) with only low dose rapamycin immunosuppression therapy (0.1 mg kg⁻¹ d⁻¹ i.p. for 14 days post-transplant). This suggests that selective HDAC10i can be useful therapeutic immunosuppression/immunomodulation agents for inflammatory disorders including colitis and also for transplantation.⁴⁶

Recently, Christianson and co-workers solved the x-ray crystal structure of zebrafish HDAC10 (zHDAC10).⁶ They elegantly demonstrated that both the zebrafish and human HDAC10 (hHDAC10) enzymes are highly active polyamine deacetylases (PDAC), while being poor lysine deacetylases. Therefore, it appears as if HDAC10 may not act on proteins, but on polyamine metabolites, e.g. spermidine and putrescine. Two specific structural features near the active site of the enzyme(s) were identified as being responsible for PDAC activity. First, the negatively charged Glu272 (hHDAC10 numbering) amino acid was demonstrated to be a gatekeeper residue, which establishes specificity for cationic polyamine substrates over acetylated lysines. In all other HDAC isozymes except the first catalytic domain of HDAC6, this amino acid is hydrophobic, usually a leucine. Second, the L1 loop of HDAC10 contains a two-residue insertion relative to HDAC6 in both zebrafish and humans. Christianson et al. found that, in zHDAC10, these inserted residues plus a two-residue mutation create a unique 310-helix that constricts the active site, making an acetylated lysine side chain, but not an acetylated polyamine, too short to reach the active site zinc atom. In hHDAC10, these four residues, numbered 21-24, are Pro-Glu-Cys-Glu. Both an E272L HDAC10 mutant and a mutant lacking the two-residue loop insertion were found to have increased HDAC activity and diminished PDAC activity in enzymatic assays. Interestingly, this model suggests that bulky (e.g. 8) Class IIB inhibitors cannot fit into the constricted binding pocket of HDAC10 without significant movement of the L1 loop. The fact that an E272L mutation alone is sufficient to enable deacetylation of lysines, however, suggests that the L1 loop may have this flexibility.

Based on that work, the X-ray crystal structures of zebrafish HDAC10 complexed with eight different analogues of N⁸-acetylspermidine have been determined, and it has been suggested that the interactions between residues in the different N⁸-acetylspermidine compound and HDAC10 may be useful for the future design of compounds selective for HDAC10 inhibition.⁴⁴

After it had been found that some known HDAC inhibitors bind HDAC10 as well,^{38_40} Géraldy et al. could show an unexpectedly potent HDAC10 binding of tubastatin A, which had previously been described as a highly selective HDAC6 inhibitor.⁴¹ Analysis of a targeted selection of tubastatin A derivatives revealed that a basic amine in the cap group was required for strong HDAC10, but not HDAC6, binding. Only potent HDAC10 binders mimicked HDAC10 knockdown by causing dose-dependent accumulation of acidic vesicles in the BE(2)-C neuroblastoma cell line. Docking of inhibitors into human HDAC10 homology models indicated that a hydrogen-bond between a basic cap group nitrogen and the HDAC10 gatekeeper residue Glu272 was responsible for potent HDAC10 binding.

While the work of Géraldy et al. has shown the possibility of identifying potent HDAC10 inhibitors, there was still an unmet need for finding such a potent inhibitor, which simultaneously was no longer significantly binding to HDAC6 and/or to other HDACs.

### SUMMARY OF THE INVENTION

The present invention is based on the surprising observation that variants of suberanilohydroxamic acid (SAHA), which comprise the substitution of a particular methylene group by an amino group, result in the formation of potent and specific HDAC10 inhibitors.

Thus, in a first aspect, the present invention relates to an HDAC10 inhibitor of Formula (I)

CAP-(CR^{y*}R^{y*′})ₙ—NR²—CR³R^{3′}—CR⁴R^{4′}—CR⁵R^{5′}—ZBD (I)
wherein:
CAP is a capping group selected from the groups of aryl-X— and heteroaryl-X—, wherein X is absent or is selected from —C(═O)—NR¹—, —NR—C(═O)—, —S(═O)₂—NR¹—, —NR—S(═O)₂—, —S(═O)(═NR)—NR′—, —NR—S(═O)(═NR)—, —C(═NR)—, —O—, —S—, —S(═O)—, —S(═O)₂—, and —C(═O)—,
wherein
R and R¹ are each independently a residue selected from H and a substituent selected from linear or branched C₁₋₄-alkyl, cyclopropyl, benzyl, aryl and heteroaryl, wherein said substituent is optionally further substituted, in particular by a further substituent selected from the list of —F, —OH, —OR, and —NR₂,
n is an integer selected from 1, 2 and 3;
y is an integer taking the values from the range of 1 to n;
ZBD is a zinc-binding domain selected from the group of:
—C(═O)—NH—OH, —C(═S)—NH—OH, —C(═N—OH)—NHOH, —C(═O)NH—R⁶, —C(═N—OH)—C(═O)NH—R⁶, —C(═O)CF₃, —C(═O)CH₂SH, C(═S)CH₂SH, —SH, —C(═NH)—NH—OH, and —C(═N—OH)—NH₂, wherein R⁶ is a residue selected from H, linear or branched C₁₋₄-alkyl, -cyclopropyl, and benzyl;
and
R² is a residue selected from H and a substituent selected from linear or branched C₁₋₄-alkyl, cyclopropyl, benzyl, aryl and heteroaryl, wherein said substituent is optionally further substituted, in particular by a further substituent selected from the list of —F, —OH, —OR, and —NR₂, and each R^{y*}, each R^{y*′}, R³, R^{3′}, R⁴, R^{4′}, R⁵, and R^{5′} are independently selected from residues H, CH₃, F, CFH₂, CF₂H and CF₃, provided that in total not more than three of said residues are different from —H;
or
two residues selected from the R¹, R^{y*}, R^{y*′} and R² residues, together with the atoms they are attached to, form a three to six-membered ring, wherein said three to six-membered ring is optionally further substituted, in particular by a further substituent selected from the list of —F, —OH, —OR, and —NR₂, and the remaining residues R¹, each R^{y*}, each R^{y*′}, R³, R^{3′}, R⁴, R^{4′}, R⁵, and R^{5′} are independently selected from residues H, CH₃, F, CFH₂, CF₂H and CF₃, provided that in total not more than three of said residues are different from —H;
or
two residues selected from the R², R³, R^{3′}, R⁴, R⁵, and R^{5″} residues, together with the atoms they are attached to, form a three to six-membered ring, and the remaining residues R¹, each R^{y*}, each R³, R^{3′}, R⁴, R^{4′}, R⁵, and R^{5′} are independently selected from residues H, CH₃, F, CFH₂, CF₂H and CF₃, provided that in total not more than three of said residues are different from —H.

In a second aspect, the present invention relates to a pharmaceutically acceptable salt form of the HDAC10 inhibitor of the present invention.

In a third aspect, the present invention relates to a pharmaceutical composition comprising an HDAC10 inhibitor of the present invention, or a pharmaceutically acceptable salt form of an HDAC10 inhibitor of the present invention.

In a fourth aspect, the present invention relates to an HDAC10 inhibitor of the present invention, a pharmaceutically acceptable salt form of an HDAC10 inhibitor of the present invention or a pharmaceutical composition of the present invention, for use in the treatment of a disease, such as cancer, autoimmune disorders or neurodegeneration, in particular cancer, of for use in organ transplantation.

In a fifth aspect, the present invention relates to a method of treating a disease, such as cancer, autoimmune disorders or neurodegeneration, comprising the step of administering an HDAC10 inhibitor of the present invention, a pharmaceutically acceptable salt form of an HDAC10 inhibitor of the present invention, or a pharmaceutical composition of the present invention to a patient suffering from said disease, in particular cancer.

In a sixth aspect, the present invention relates to a method of preventing donor organ rejection, comprising the step of administering an HDAC10 inhibitor of the present invention, a pharmaceutically acceptable salt form of an HDAC10 inhibitor of the present invention, or a pharmaceutical composition of the present invention to a patient after organ transplantation.

### FIGURE

FIG. 1 shows the structures of different HDAC inhibitors.

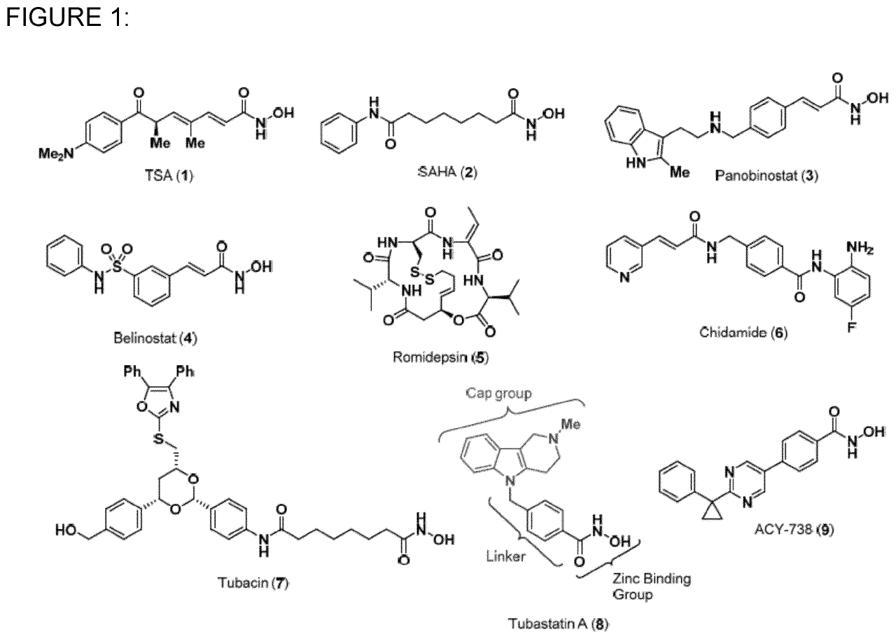

### DETAILED DESCRIPTION OF THE INVENTION

In a first aspect, the present invention relates to an HDAC10 inhibitor of Formula (I)

CAP-(CR^{y*}R^{y*′})ₙ—NR²—CR³R^{3′}—CR⁴R^{4′}—CR⁵R^{5′}—ZBD (I)
wherein:
CAP is a capping group selected from the groups of aryl-X— and heteroaryl-X—, wherein X is absent or is selected from —C(═O)—NR¹—, —NR—C(═O)—, —S(═O)₂—NR¹—, —NR—S(═O)₂—, —S(═O)(═NR)—NR′—, —NR—S(═O)(═NR)—, —C(═NR)—, —O—, —S—, —S(═O)—, —S(═O)₂—, and —C(═O)—,
wherein
R and R¹ are each independently a residue selected from H and a substituent selected from linear or branched C₁₋₄-alkyl, cyclopropyl, benzyl, aryl and heteroaryl, wherein said substituent is optionally further substituted, in particular by a further substituent selected from the list of —F, —OH, —OR, and —NR₂,
n is an integer selected from 1, 2 and 3;
y is an integer taking the values from the range of 1 to n;
ZBD is a zinc-binding domain selected from the group of:
—C(═O)—NH—OH, —C(═S)—NH—OH, —C(═N—OH)—NHOH, —C(═O)NH—R⁶, —C(═N—OH)—C(═O)NH—R⁶, —C(═O)CF₃, —C(═O)CH₂SH, C(═S)CH₂SH, —SH, —C(═NH)—NH—OH, and —C(═N—OH)—NH₂, wherein R⁶ is a residue selected from H, linear or branched C₁₋₄-alkyl, -cyclopropyl, and benzyl;
and
R² is a residue selected from H and a substituent selected from linear or branched C₁₋₄-alkyl, cyclopropyl, benzyl, aryl and heteroaryl, wherein said substituent is optionally further substituted, in particular by a further substituent selected from the list of —F, —OH, —OR, and —NR₂, and each R^{y*}, each R^{y*′}, R³, R^{3′}, R⁴, R^{4′}, R⁵, and R^{5′} are independently selected from residues H, CH₃, F, CFH₂, CF₂H and CF₃, provided that in total not more than three of said residues are different from —H;
or
two residues selected from the R¹, R^{y*}, R^{y*′} and R² residues, together with the atoms they are attached to, form a three to six-membered ring, wherein said three to six-membered ring is optionally further substituted, in particular by a further substituent selected from the list of —F, —OH, —OR, and —NR₂, and the remaining residues R¹, each R^{y*}, each R^{y*′}, R³, R^{3′}, R⁴, R^{4′}, R⁵, and R^{5′} are independently selected from residues H, CH₃, F, CFH₂, CF₂H and CF₃, provided that in total not more than three of said residues are different from —H;
or
two residues selected from the R², R³, R^{3′}, R⁴, R^{4′}, R⁵, and R^{5″} residues, together with the atoms they are attached to, form a three to six-membered ring, and the remaining residues R¹, each R^{y*}, each R^{y*′} R³, R^{3′}, R⁴, R^{4′}, R⁵, and R^{5′} are independently selected from residues H, CH₃, F, CFH₂, CF₂H and CF₃, provided that in total not more than three of said residues are different from —H.

In particular embodiments, ZBD is selected from —C(═O)—NH—OH, —C(═N—OH)—C(═O)NH—R⁶, —C(═O)CF₃, —C(═O)CH₂SH, —SH, —C(═NH)—NH—OH, and —C(═N—OH)—NH₂, wherein R⁶ is a residue selected from H, linear or branched C₁₋₄-alkyl, -cyclopropyl, and benzyl;

In an alternative first aspect, the present invention relates to an HDAC10 inhibitor of Formula (Ia)

CAP-(CR^{y*}R^{y*′})ₙ—NR²—CR³R^{3′}—CR⁴R^{4′}—CR⁵R^{5′}—ZBD (Ia)
wherein:
CAP is a capping group selected from the groups of aryl-X— and heteroaryl-X—, wherein X is —CH₂—NR¹—,
and wherein the other residues are as defined in the first aspect.

In a second alternative aspect, the present invention relates to an HDAC10 inhibitor of Formula (Ib)

CAP-(CR^{y*}R^{y*′})ₙ—NR²—CR³R^{3′}—CR⁴R^{4′}—CR⁵R^{5′}—ZBD (Ib)
wherein:
CAP is a capping group selected from the groups of aryl-X— and heteroaryl-X—, wherein X is absent or is selected from —C(═O)—NR¹—, —NR—C(═O)—, —S(═O)₂—NR¹—, —NR—S(═O)₂—, —S(═O)(═NR)—NR′—, —NR—S(═O)(═NR)—, —C(═NR)—, —O—, —S—, —S(═O)—, —S(═O)₂—, —C(═O)—, and —CH₂—NR¹—,
wherein
R and R¹ are each independently a residue selected from H and a substituent selected from linear or branched C₁₋₄-alkyl, cyclopropyl, benzyl, aryl and heteroaryl, wherein said substituent is optionally further substituted, in particular by a further substituent selected from the list of —F, —OH, —OR, and —NR₂;
n is an integer selected from 1, 2 and 3;
y is an integer taking the values from the range of 1 to n;
ZBD is a zinc-binding domain selected from the group of:
—C(═O)—NH—OH, —C(═S)—NH—OH, —C(═N—OH)—NHOH, —C(═O)NH—R⁶, —C(═N—OH)—C(═O)NH—R⁶, —C(═O)CF₃, —C(═O)CH₂SH, C(═S)CH₂SH, —C(═NH)—NH—OH, and —C(═N—OH)—NH₂, wherein R⁶ is a residue selected from H, linear or branched C₁₋₄-alkyl, -cyclopropyl, and benzyl, and
and
R² and R⁵ are each a residue independently selected from H and a substituent selected from linear or branched C₁₋₄-alkyl, cyclopropyl, benzyl, aryl and heteroaryl, wherein said substituent is optionally further substituted, in particular by a further substituent selected from the list of —F, —OH, —OR, and —NR₂, and each R^{y*}, each R^{y*′}, R³, R^{3′}, R⁴, and R^{4′} are independently selected from residues H, CH₃, F, CFH₂, CF₂H and CF₃, provided that in total not more than three of said residues are different from —H;
or
two residues selected from the R¹, R^{y*}, R^{y*′} and R² residues, together with the atoms they are attached to, form a three to six-membered ring, wherein said three to six-membered ring is optionally further substituted, in particular by a further substituent selected from the list of —F, —OH, —OR, and —NR₂, and the remaining residues R¹, each R^{y*}, each R^{y*′}, R³, R^{3′}, R⁴, and R^{4′} are independently selected from residues H, CH₃, F, CFH₂, CF₂H and CF₃, provided that in total not more than three of said residues are different from —H;
or
two residues selected from the R², R³, R^{3′}, R⁴, R^{4′}, and R⁵ residues, together with the atoms they are attached to, form a three to six-membered ring, and the remaining residues R¹, each R^{y*}, each R^{y*′} R³, R^{3′}, R⁴, R^{4′}, and R⁵ are independently selected from residues H, CH₃, F, CFH₂, CF₂H and CF₃, provided that in total not more than three of said residues are different from —H.

The fact that structures according to formulae (I), (Ia) and/or (Ib) exhibit selectivity for HDAC10 could not have been expected. In the prior art, the design and synthesis of a series of HDAC inhibitors has been described, including compounds of generic structure A, which were tested in an HDAC1 inhibition assay, a cell proliferation inhibition assay, and in an in vivo anticancer assay.⁴³ Apparently, no additional HDACs were tested, and no data on HDAC selectivity and/or specificity are available.

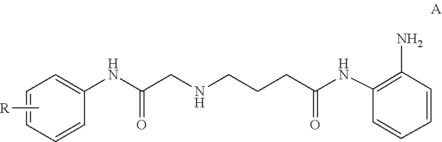

The present invention is intended to include all isotopes of atoms occurring on the present compound. Isotopes are atoms having the same atomic number but different mass numbers. By way of general example and without limitation, isotopes of hydrogen include deuterium and tritium, in particular deuterium. Isotopes of carbon include ¹²C and ¹⁴C.

In the context of the present invention, the term “aryl” is intended to mean a ring or ring system being part of any stable monocyclic or polycyclic system, where such ring or ring system has between 3 and about 20 carbon atoms, but has no heteroatom, and where the ring or ring system consists of an aromatic moiety as defined by the “(4n+2)π electron rule”. For the sake of clarity, if a substituent is a polycyclic system wherein one ring or ring system comprised in said polycyclic system consists of an aromatic moiety as defined herein, then such substituent will be referred to as “aryl”, if substitution occurs via said aromatic moiety. This includes, but is not limited to, phenyl and fused benzene ring systems, for example, naphthalene, anthracene, or phenanthrene ring systems, or, for example, a benzene ring fused to one or more cycloalkyl moieties to form, for example, indanyl, fluorenyl or tetrahydronaphthyl (tetralin), or fused to one or more heterocycloalklyl rings, e.g. as in indolenyl, provided, however, that in each such case, such fused system is linked as a substituent via the aromatic moiety. As used herein, the term “heteroaryl” refers to a ring or ring system being part of any stable mono- or polycyclic system, where such ring or ring system has between 3 and about 20 atoms, which ring or ring system consists of an aromatic moiety as defined by the “(4n+2)π electron rule” and which contains carbon atoms and one or more nitrogen, sulfur, and/or oxygen heteroatoms. For the sake of clarity, if a substituent is a polycyclic system wherein one ring or ring system comprised in said polycyclic system consists of an aromatic moiety containing a heteroatom as defined herein, then such substituent will be referred to as “heteroaryl”, if substitution occurs via the aromatic moiety containing the heteroatom. In certain embodiments, the total number of N, S and O atoms in the heteroaryl is between 1 and about 4. In certain embodiments, the total number of S and O atoms in the aromatic heteroaryl is not more than 1. In certain embodiments, a nitrogen in the heterocycle may be quaternized or oxidized to an N-oxide. Examples of heteroaryls include, but are not limited to, pyrrolyl, pyrazolyl, imidazolyl, indolyl, benzimidazolyl, furanyl, benzofuranyl, thiophenyl, benzothiophenyl, pyridinyl, pyrimidinyl, pyrazinyl, triazinyl, quinolinyl, quinazolinyl. Also included in the term heteroaryl are fused heteroaryls containing, for example, the above heteroaryls fused to cycloalkyls or heterocycloalkyls (provided, in each case, that such fused system is linked as a substituent via the aromatic moiety containing at least one heteroatom).

In the context of the present invention, the term “substituted” is intended to indicate that one or more hydrogens on the atom or group indicated in the expression using “substituted” is replaced with a selection from the indicated group(s), provided that the indicated atom's normal valency, or that of the appropriate atom of the group that is substituted, is not exceeded, and that the substitution results in a stable compound. The terms “substituted or unsubstituted” or “optionally substituted” are intended to mean that a given compound, or substructure of a compound, is either unsubstituted, or substituted, as defined herein, with one or more substituents as indicated.

In one embodiment of the present invention, the compound of the invention has a purity of more than 90%, more than 95%, more than 98%, or more than 99%. The compound may exist in one or more crystalline forms, including two or more polymorphic forms, and may exist as a dry solid or as a solvate including a defined amount of a solvent, including a hydrate including defined amounts of water.

In another embodiment, the compound of the invention is the planned and deliberate product of a synthetic chemistry scheme, i.e., produced by specific and planned chemical processes conducted in reaction vessels, and not by degradation, metabolism or fermentation, or produced as impurity or by-product in the synthesis of other compounds.

In certain embodiments, the compound of the invention is purified or isolated, e.g., to have a purity of at least 80%, preferably at least 90%, more preferably at least 95%, such as at least 97%, at least 98% or even at least 99%. Purity, as used herein, can refer to either absolute or relative purity. Absolute purity refers to the amount of the compound of the invention obtained as the product of a synthetic chemistry scheme, either before or after one or more purification steps. Relative purity refers to the amount of the compound of the invention relative to one or more impurities such as by-products, degradation products (e.g., metabolites, products of oxidation or hydrolysis, etc.) and/or compounds that degrade to form the compound of the invention (e.g., precursors or prodrugs), e.g., that may be present in the product of a synthetic chemistry scheme. Thus, absolute purity refers to the amount of a compound relative to all others, while relative purity is generally unaffected by the addition of unrelated compounds, such as excipients, stabilizers, or other medicaments for conjoint administration. Purity can be assessed based upon weight, volume or molar ratios of one compound relative to others. Purity can be measured by a variety of analytical techniques, including elemental abundance, UV-visible spectrometry, HPLC, GC-MS, NMR, mass spectrometry, and thin layer chromatography, preferably by HPLC, GC-MS, or NMR.

In particular embodiments, CAP is a capping group selected from the group of: aryl-C(═O)—NH—, heteroaryl-C(═O)—NH—, and aryl-NH—C(═O)—, and heteroaryl-NH—C(═O)—.

In particular such embodiments, CAP is a capping group selected from the group of phenyl-NH—C(═O)—, phenyl-C(═O)—NH—, 1-naphthyl-C(═O)—NH—, and 7-indazolyl-C(═O)—NH—. In particular embodiments, CAP is phenyl-NH—C(═O)— or phenyl-C(═O)—NH—. In particular embodiments, CAP is phenyl-NH—C(═O)—. In other particular embodiments, CAP is phenyl-C(═O)—NH—.

In particular embodiments, R¹ and the aryl or heteroaryl group of the CAP group, together with the atoms they are attached to, form a five- or six-membered ring. In particular such embodiments, CAP is benzimidazol-2-yl.

In particular embodiments, n is an integer selected from 2 and 3, in particular n is 2.

In particular embodiments, R¹ is a residue selected from H and a substituent selected from linear or branched C₁₋₄-alkyl, cyclopropyl, benzyl, aryl and heteroaryl, wherein said substituent is optionally further substituted, in particular by a further substituent selected from the list of —F, —OH, —OR, and —NR₂.

In particular embodiments, R² is a residue selected from H, methyl, ethyl, n-propyl, i-propyl, n-butyl, cyclopropyl, and benzyl. In a particular embodiment, R² is methyl.

In particular embodiments, each R^{y*}, each R^{y*′}, R³, R^{3′}, R⁴, R^{4′}, R⁵, and R^{5′} are each —H.

In particular embodiments, ZBD is —C(═O)—NH—OH. In particular such embodiments, the HDAC10 inhibitor is of Formula (I) or (Ia).

In particular other embodiments, ZBD is —C(═O)CH₂SH. In particular such embodiments, the HDAC10 inhibitor is of Formula (Ib).

In particular embodiments, where CAP is phenyl-NH—C(═O)—, n is 2, each R^{y*}, each R^{y*′}, R³, R^{3′}, R⁴, R^{4′}, R⁵, and R^{5′} are each —H and ZBD is —C(═O)—NH—OH, R² is different from —H. In particular other embodiments, where CAP is phenyl-C(═O)—NH—, n is 2, each R^{y*}, each R^{y*′}, R³, R^{3′}, R⁴, R^{4′}, R⁵, and R^{5′} are each —H and ZBD is —C(═O)—NH—OH, R² is different from —H.

In particular embodiments, the HDAC10 inhibitor is selected from the group of DKFZ-711, DKFZ-775, DKFZ-772, DKFZ-728, DKFZ-777, DKFZ-773, DKFZ-748, DKFZ-750, DKFZ-757, DKFZ-771, DKFZ-774, DKFZ-746, DKFZ-747, DKFZ-749, DKFZ-751, DKFZ-752, DKFZ-753, DKFZ-754, DKFZ-755, DKFZ-756, DKFZ-769, DKFZ-776, DKFZ-758, DKFZ-759, DKFZ-767, DKFZ-770, DKFZ-714, DKFZ-715, DKFZ-716, DKFZ-717, DKFZ-718, DKFZ-724, DH22, DH25, DH35. DH40, DH53, DH67, DH71, DH79, DH88, and DKFZ-825. In particular embodiments, the HDAC10 inhibitor is selected from the group of DKFZ-711, DKFZ-714, DKFZ-715, DKFZ-716, DKFZ-717, DKFZ-718, DKFZ-724, and DKFZ-728.

In particular embodiments, the two residues R² and R⁵, together with the atoms they are attached to, form a six-membered ring, and the remaining residues R¹, each R^{y*}, each R^{y*′} R³, R^{3′}, R⁴, R^{4′}, R⁵, and R^{5′} are independently selected from residues H, CH₃, F, CFH₂, CF₂H and CF₃, provided that in total not more than three of said residues are different from —H.

In particular such embodiments, all remaining residues R¹, each R^{y*}, each R^{y*′} R³, R^{3′}, R⁴, R^{4′}, R⁵, and R^{5′} are H.

In a second aspect, the present invention relates to a pharmaceutically acceptable salt form of the HDAC10 inhibitor of the present invention.

As used herein, “pharmaceutically acceptable salts” refers to derivatives of the disclosed compound wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. Lists of suitable salts are found in Remington's Pharmaceutical Sciences, 18th ed., Mack Publishing Company, Easton, Pa., 1990, p. 1445, or in PH Stahl und CG Wermuth (eds.), Handbook of Pharmaceutical Salts: Eigenschaften, Auswahl und Verwendung, Weinheim/Zürich: Wiley-VCH/VHCA, 2002, the disclosure of which is hereby incorporated by reference. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, palmoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The pharmaceutically acceptable salts of the present invention can be synthesized from a parent compound which contains a basic or acidic moiety by conventional chemical methods. Generally, such salts can be prepared by reacting the free acid or base forms of the compound with a stoichiometric amount of the appropriate base or acid in water or in an organic solvent, or in a mixture of the two; generally, non-aqueous media like ether, EtOAc, ethanol, isopropanol, or acetonitrile are preferred.

Any salt that retains the desired biological activity of the compound contained herein and that exhibits minimal or no undesired or toxicological effects is intended for inclusion here. Pharmaceutically acceptable salts include those derived from pharmaceutically acceptable organic or inorganic acids and bases. Non-pharmaceutically acceptable acids and bases also find use herein, as for example, in the synthesis and/or purification of the compound of interest. Thus, all “salts” are also encompassed within the scope of the instant invention.

Non-limiting examples of suitable salts include those derived from inorganic acids, such as, for example, hydrochloric acid, hydrobromic acid, sulfuric acid, phosphoric acid, nitric acid, bicarbonic acid, carbonic acid; and salts formed with organic acids, such as, for example, formic acid, acetic acid, oxalic acid, tartaric acid, succinic acid, malic acid, malonic acid, ascorbic acid, citric acid, benzoic acid, tannic acid, palmoic acid, alginic acid, polyglutamic acid, tosic acid, methanesulfonic acid, naphthalenesulfonic acid, naphthalenedisulfonic acid, α-ketoglutaric acid, ß-glycerophosphoric acid and polygalacturonic acid.

Suitable salts obtained by reacting an acidic compound with a base include those derived from alkali metals such as lithium, potassium and sodium, from alkaline earth metals such as calcium and magnesium, as well as from other acids well known to those of skill in the pharmaceutical art. Other suitable salts include those derived from metal cations such as zinc, bismuth, barium, or aluminum, or with a cation formed from an amine, such as ammonia, N,N-dibenzylethylene-diamine, D-glucosamine, tetraethylammonium, or ethylenediamine. Moreover, suitable salts include those derived from a combination of acids and bases, such as, for example, a zinc tannate salt.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

In particular embodiments, the HDAC10 of the present invention is reacted with an acid selected from the group of: hydrochloric, hydrobromic, sulfuric, sulfamic, phosphoric, nitric; acetic, propionic, succinic, glycolic, stearic, lactic, malic, tartaric, citric, ascorbic, palmoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic acid, in particular hydrochloric acid.

In a third aspect, the present invention relates to a pharmaceutical composition comprising an HDAC10 inhibitor of the present invention, or a pharmaceutically acceptable salt form of an HDAC10 inhibitor of the present invention.

The compound of this invention can be formulated and administered to treat individuals in need by any means that produces contact of the active ingredient with the agent's site of action, such as a cell, in the body of an individual. It can be administered by any conventional means available for use in conjunction with pharmaceuticals, either as individual therapeutic active ingredients or in a combination of therapeutic active ingredients. It can be administered alone, but are generally administered with a pharmaceutically acceptable diluent, excipient or carrier selected on the basis of the chosen route of administration and standard pharmaceutical practice.

A pharmaceutical composition comprising less than a therapeutically effective amount of the compound described above, or a prodrug thereof, may also be used, such as when used in combination with another pharmaceutical composition, such as an anti-cancer agent, so that such combination is therapeutically effective, or may be useful for prophylactic treatment.

Pharmaceutical compositions for use in accordance with the present invention may be formulated in conventional manner using one or more pharmaceutically acceptable diluents, excipients or carriers. The pharmaceutical compositions of the invention can be formulated for a variety of routes of administration, including systemic and topical or localized administration. Techniques and formulations generally may be found in Remington's Pharmaceutical Sciences, Meade Publishing Co., Easton, Pa. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, capsules, boluses, powders, granules, pastes for application to the tongue; (2) parenteral administration, for example, by subcutaneous, intramuscular or intravenous injection as, for example, a sterile solution or suspension; (3) topical application, for example, as a cream, ointment or spray applied to the skin; or (4) intravaginally or intrarectally, for example, as a pessary, cream or foam. In certain embodiments, the pharmaceutical preparations may be non-pyrogenic, i.e., do not substantially elevate the body temperature of a patient.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, as well as the particular mode of administration. The amount of active ingredient which can be combined with a carrier material to produce a single dosage form will generally be that amount of inhibitor which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association the compound of the present invention with the carrier and, optionally, one or more accessory ingredients. In general, the formulations are prepared by uniformly and intimately bringing into association the compound of the present invention with liquid carriers, or finely divided solid carriers, or both, and then, if necessary, shaping the product.

For systemic administration, injection is preferred, including intramuscular, intravenous, intraperitoneal, and subcutaneous (i.m., i.v., i.p., and s.c. respectively). The phrases “systemic administration”, “administered systemically”, “peripheral administration”, and “administered peripherally” as used herein mean the administration of a compound, drug or other material other than directly into the central nervous system, such that it enters the patient's system and, thus, is subject to metabolism and other like processes, for example, subcutaneous administration.

For injection, the pharmaceutical compositions of the invention can be formulated in liquid solutions, preferably in physiologically compatible buffers such as Hank's solution or Ringer's solution. In addition, the pharmaceutical compositions may be formulated in solid form and redissolved or suspended immediately prior to use. Lyophilized forms are also included.

Pharmaceutical compositions of the invention may be formulated to be suitable for oral administration may be in the form of capsules, cachets, sachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of the compound of the present invention as an active ingredient. The compound of the present invention may also be administered as a bolus, electuary or paste.

In formulating the pharmaceutical compositions of the invention in solid dosage forms for oral (p.o.) administration (capsules, tablets, pills, dragees, powders, granules and the like), the compound of the invention as active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, high molecular weight polyethylene glycols, and the like.

Gelatin capsules contain the compound of the present invention as active ingredient and powdered carriers, such as lactose, starch, cellulose derivatives, magnesium stearate, stearic acid, and the like. Similar carriers can be used to make compressed tablets. Both tablets and capsules can be manufactured as sustained release products to provide for continuous release of medication over a period of h. Compressed tablets can be sugar-coated or film-coated to mask any unpleasant taste and protect the tablet from the atmosphere, or enteric coated for selective disintegration in the gastrointestinal tract. Solid compositions of a similar type are also employed as fillers in soft and hard-filled gelatin capsules; preferred materials in this connection also include lactose or milk sugar as well as high molecular weight polyethylene glycols. A preferred formulation is a solution or suspension in an oil, for example olive oil, Miglyol, or Capmul, in a soft gelatin capsule. Antioxidants may be added to prevent long-term degradation as appropriate.

A tablet may be made by compression or molding, optionally with one or more accessory ingredients. Compressed tablets may be prepared using a binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered inhibitor moistened with an inert liquid diluent.

The tablets and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulations so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the pharmaceutical compositions of the invention include pharmaceutically acceptable emulsions, microemulsions, solutions, suspensions, syrups, and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the pharmaceutical compositions for oral administration can also include adjuvants such as wetting agents, emulsifying and suspending agents, sweetening, flavoring, coloring, perfuming, and preservative agents.

Suspensions, in addition to the pharmaceutical composition of the present invention, may contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

For buccal administration the pharmaceutical compositions may take the form of tablets or lozenges formulated in a conventional manner.

For administration by inhalation, the pharmaceutical compositions of the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, for example, gelatin for use in an inhaler or insufflator may be formulated containing a powder mix of the therapeutic agents and a suitable powder base such as lactose or starch.

The pharmaceutical compositions may be formulated for parenteral administration by injection, e.g., by bolus injection or continuous infusion. Formulations for injection may be presented in unit dosage form, e.g., in ampoules or in multi-dose containers, with an added preservative. The pharmaceutical compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

Pharmaceutical compositions of this invention suitable for parenteral administration comprise one or more inhibitors of the invention in combination with one or more pharmaceutically acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These pharmaceutical compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the pharmaceutical compositions. In addition, prolonged absorption of the injectable pharmaceutical form may be brought about by the inclusion of agents that delay absorption such as aluminum monostearate and/or gelatin.

In addition to the formulations described previously, the pharmaceutical compositions may also be formulated as a depot preparation. Such long acting formulations may be administered by implantation (for example subcutaneously or intramuscularly) or by intramuscular injection. Thus, for example, the pharmaceutical compositions may be formulated with suitable polymeric or hydrophobic materials (for example as an emulsion in an acceptable oil) or ion exchange resins, or as sparingly soluble derivatives, for example, as a sparingly soluble salt.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents may be used to facilitate permeation. Transmucosal administration may be through nasal sprays or using suppositories. For topical administration, the pharmaceutical compositions of the invention are formulated into ointments, salves, gels, or creams as generally known in the art. A wash solution can be used locally to treat an injury or inflammation to accelerate healing.

In some cases, in order to prolong the therapeutic effect of an inhibitor, it is desirable to slow the absorption of the inhibitor from subcutaneous or intramuscular injection. This may be accomplished by the use of a liquid suspension of crystalline or amorphous material having poor water solubility. The rate of absorption of the inhibitor then depends upon its rate of dissolution which, in turn, may depend upon crystal size and crystalline form. Alternatively, delayed absorption of a parenterally administered inhibitor form is accomplished by dissolving or suspending the inhibitor in an oil vehicle.

Pharmaceutical compositions of the invention may be formulated for rectal or vaginal administration as a suppository, which may be prepared by mixing the compound of the invention with one or more suitable nonirritating excipients or carriers comprising, for example, cocoa butter, polyethylene glycol, a suppository wax or a salicylate, and which is solid at rt, but liquid at body temperature and, therefore, will melt in the rectum or vaginal cavity and release the active inhibitor.

Formulations of the pharmaceutical compositions of the present invention, which are suitable for vaginal administration, also include pessaries, tampons, creams, gels, pastes, foams or spray formulations containing such carriers as are known in the art to be appropriate.

Dosage forms for the topical or transdermal administration of the compound of this invention include powders, sprays, ointments, pastes, creams, lotions, gels, solutions, patches and inhalants. Such compound may be mixed under sterile conditions with a pharmaceutically acceptable carrier, and with any preservatives, buffers, or propellants which may be required.

The ointments, pastes, creams and gels may contain, in addition to the compound of the invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of the compound of the present invention to the body. Such dosage forms can be made by dissolving or dispersing an inhibitor of the present invention in the proper medium. Absorption enhancers can also be used to increase the flux of the drug across the skin. The rate of such flux can be controlled by either providing a rate controlling membrane or dispersing the compound of the present invention in a polymer matrix or gel.

Ophthalmic formulations, eye ointments, powders, solutions and the like, are also contemplated as being within the scope of this invention.

The pharmaceutical compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. In other embodiments, the pack or dispenser may be further packaged in an outer carton.

A pharmaceutical composition of the present invention can also be formulated as a sustained and/or timed release formulation. Such sustained and/or timed release formulations may be made by sustained release means or delivery devices that are well known to those of ordinary skill in the art, such as those described in U.S. Pat. Nos. 3,845,770; 3,916,899; 3,536,809; 3,598,123; 4,008,719; 4,710,384; 5,674,533; 5,059,595; 5,591,767; 5,120,548; 5,073,543; 5,639,476; 5,354,556; and 5,733,566, the disclosures of which are each incorporated herein by reference. The pharmaceutical compositions of the present invention can be used to provide slow or sustained release of one or more of the active ingredients using, for example, hydroxypropylmethyl cellulose, other polymer matrices, gels, permeable membranes, osmotic systems, multilayer coatings, microparticles, liposomes, microspheres, or the like, or a combination thereof to provide the desired release profile in varying proportions. Suitable sustained release formulations known to those of ordinary skill in the art, including those described herein, may be readily selected for use with the pharmaceutical compositions of the invention. Thus, single unit dosage forms suitable for oral administration, such as, but not limited to, tablets, capsules, gelcaps, caplets, powders, and the like, that are adapted for sustained release are encompassed by the present invention.

Injectable depot forms are made by forming microencapsuled matrices of the subject inhibitors in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

In a fourth aspect, the present invention relates to an HDAC10 inhibitor of the present invention, a pharmaceutically acceptable salt form of an HDAC10 inhibitor of the present invention or a pharmaceutical composition of the present invention, for use in the treatment of a disease, such as cancer, autoimmune disorders or neurodegeneration, in particular cancer, of for use in organ transplantation.

In particular embodiments, autophagy is upregulated in the cells of said cancer.

In a fifth aspect, the present invention relates to a method of treating a disease, such as cancer, autoimmune disorders or neurodegeneration, in particular cancer, comprising the step of administering an HDAC10 inhibitor of the present invention, a pharmaceutically acceptable salt form of an HDAC10 inhibitor of the present invention, or a pharmaceutical composition of the present invention to a patient suffering from said disease cancer.

In particular embodiments, autophagy is upregulated in the cells of said cancer.

In a sixth aspect, the present invention relates to a method of preventing donor organ rejection, comprising the step of administering an HDAC10 inhibitor of the present invention, a pharmaceutically acceptable salt form of an HDAC10 inhibitor of the present invention, or a pharmaceutical composition of the present invention to a patient after organ transplantation.

When the compound of the present invention are administered as pharmaceuticals, to individuals, such as humans and animals, they can be given per se or as a pharmaceutical composition containing, for example, 0.1 to 99.5% (in certain embodiments, 0.5 to 90%) of active ingredient in combination with a pharmaceutically acceptable carrier.

The present invention provides new methods of treating proliferative, degenerative and other disorders or diseases, including cancer, autoimmune disorders or neurodegeneration, in particular cancer, or methods of using such novel inhibitors in organ transplantation, by administering an amount such as a therapeutically effective amount of the compound disclosed herein or a prodrug, tautomeric, pharmaceutically acceptable salt, N-oxide or stereoisomeric form thereof. The present invention further provides methods of treating proliferative, degenerative or other disorders or diseases, including cancer, autoimmune disorders or neurodegeneration, in particular cancer, or methods of using such novel inhibitors in organ transplantation, by administering a therapeutically effective combination of at least the compound disclosed herein and another anti-cancer or anti-proliferative agent.

The compound of the present invention may be administered as a salt or prodrug that, upon administration to the individual, is capable of providing directly or indirectly the parent compound, such as the compound as defined herein, or that exhibits activity itself. Non-limiting examples include a pharmaceutically acceptable salt, alternatively referred to as a “physiologically acceptable salt”. In addition, modifications made to the compound can affect its biological activity, in some cases increasing the activity over the parent compound. This activity can be assessed by preparing a salt or prodrug form of the compound, and testing its activity by using methods described herein or other methods known to those of skill in the art.

As will be apparent to a person skilled in the art, through the use of a prodrug of a given subject compound, an individual such as an animal administered or treated with such prodrug will be exposed to, and hence indirectly administered with, the subject compound. Such a procedure may expose those cells associated with a disease, such as a proliferative disease or disorder including cancer, autoimmune disorders or neurodegeneration, in particular cancer, or cells associated with donor organ rejection, to the subject compound.

All processes used to prepare the compound of the present invention and intermediates made therein are considered to be part of the present invention.

A dosage administered that will be a therapeutically effective amount of the compound sufficient, or reasonably expected by a health-care professional such as a physician, pharmacist or nurse, to result in amelioration of symptoms of, for example, the cancer or tumor will, of course, vary depending upon known factors such as the pharmacodynamic characteristics of the particular active ingredient and its mode and route of administration; age, sex, health and weight of the recipient; nature and extent of symptoms; kind of concurrent treatment, frequency of treatment and the effect desired.

The subject compound may also be administered in prophylactic treatment. If the compound is administered prior to clinical manifestation of the unwanted condition (e.g., disease or other unwanted state of the host animal) then the treatment is prophylactic (i.e., it protects the individual against initiating, developing or further developing the unwanted condition). The subject compound may also be administered to prevent a condition, disorder or diseases, such as cancer, autoimmune disorders or neurodegeneration, in particular cancer, or organ rejection after organ transplantation, or a syndrome complex, such as heart failure or any other medical condition. This includes administration of the compound the intent of which is to reduce the frequency of, or delay the onset of, symptoms of a medical condition in an individual relative to an individual which does not receive the compound. Thus, prevention of cancer includes, for example, reducing the number of detectable cancerous growths, tumors, or malignancies in a population of patients receiving a prophylactic treatment relative to an untreated control population, delaying the appearance of detectable cancerous growths in a treated population versus an untreated control population, and/or delaying disease progression and/or improving the quality of patient life, e.g., by a statistically and/or clinically significant amount.

Toxicity and therapeutic efficacy of pharmaceutical compositions of the present invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Therapeutic agents that exhibit large therapeutic indices are useful for many circumstances. In certain circumstances, even therapeutic compositions that appear to exhibit debilitating or toxic side effects may be used, including circumstances where care is taken to design a delivery system that targets such therapeutic agents to the site of affected tissue in order to minimize potential damage to unaffected cells and, thereby, reduce or localize side effects.

The data obtained from cell culture assays and animal studies can be used in formulating a range of dosage for use in humans. The dosage lies preferably within a range of circulating concentrations that include the ED₅₀ with little or no toxicity. The dosage may vary within this range depending upon the dosage form employed and the route of administration utilized. For any agents used in the method of the invention, the therapeutically effective dose can be estimated initially from cell culture assays. A dose may be formulated in animal models to achieve a circulating plasma concentration range that includes the IC₅₀ (i.e., the concentration of the test therapeutic agent which achieves a half-maximal inhibition of symptoms or inhibition of biochemical activity) as determined in cell culture. Such information can be used to more accurately determine useful doses in humans. Levels in plasma may be measured, for example, by high performance liquid chromatography.

It is understood that appropriate doses of therapeutic agents depends upon a number of factors known to those or ordinary skill in the art, e.g., a physician. The dose(s) of the subject compound will vary, for example, depending upon the identity, size, and condition of the subject or sample being treated, further depending upon the route by which the composition is to be administered, if applicable, and the effect which the practitioner desires the therapeutic to have upon the therapeutic target of targets, such as cells, nucleic acid or polypeptides, through with the disease causes, symptoms or effects are mediated.

Exemplary doses include milligram or microgram amounts of the compound of the present invention per kilogram of subject or sample weight, e.g., about 1 microgram per kilogram to about 500 milligrams per kilogram, about 100 micrograms per kilogram to about 50 milligrams per kilogram, or about 1 milligram per kilogram to about 5 milligrams per kilogram.

A person skilled in the art will appreciate that doses can also be calculated on a body surface basis. A person of 70 kg has an approximate body surface area of 1.8 square meter, and doses can be expressed as milligram or microgram amounts of the compound per body surface area of subject or sample, e.g. about 50 microgram per square meter to about 15 grams per square meter, about 5 milligrams per square meter to about 1.5 grams per square meter, or about 50 milligram per square meter to about 150 milligrams per square meter.

The present invention further provides the compound as described above for therapy. In other aspects, the invention provides the compound of the present invention for prophylactic uses.

In certain embodiments, said therapy or prophylactic use is the treatment or prevention of a proliferative disorder or disease, such as a tumor or cancer. In certain embodiments, said treatment is the treatment of a cancer that can be treated by the inhibition of the activity of a protein kinase or mutant thereof, such as the inhibition of autophagy.

Thus, the present invention additionally provides a method for treating an individual, such as a mammal, having a disease-state selected from the group of proliferative disorders or diseases, or inflammatory disorders or diseases, comprising administering to said individual a therapeutically effective amount of the compound, a prodrug, or a pharmaceutical composition of the invention as described above. In certain embodiments, said individual is a human. In certain embodiments, said proliferative disorder or disease is cancer. In certain embodiments, said treatment is the treatment of a cancer that can be treated by the inhibition of the activity of a protein kinase or mutant thereof, such as the inhibition of the activity of HDAC10.

The present invention also provides a method for prophylactic treatment of an individual such as an animal, including a mammal, particularly a human, the intent of which is to reduce the frequency of, delay the onset of, or the symptoms of a medical condition, such as cancer, in a subject relative to a subject which does not receive the composition.

In a further aspect, the invention provides methods of treating or preventing an individual suffering from a disease, such as a mammal, including a domestic mammal, cat, dog, horse, sheep, cow, rodent, and human, comprising the step of exposing said individual to an amount, including a therapeutically effective amount, of the subject compound. In certain embodiments, the disease is a proliferative disorder or disease, such as a cancer or tumour. In yet another embodiment, cells associated with said proliferative disorder or disease, including tumour cells included in a cancer, are exposed to the subject compound. In certain embodiments, said compound, or a prodrug thereof, is administered to said individual. In certain embodiments, said treatment is the treatment of a cancer that can be treated by the inhibition of the activity of a protein kinase or mutant thereof, such as the inhibition of the activity of HDAC10. In certain embodiments, the disease is an inflammatory disorder or disease. In yet another embodiment, cells associated with said inflammatory disorder or disease are exposed to the subject compound. In certain embodiments, said compound, or a prodrug thereof, is administered to said individual.

In a further aspect, the invention provides a method of killing or inhibiting proliferation or growth of a cell, comprising contacting the cell with the compound of the invention. In one embodiment, the cell is cultured in-vitro, while in an alternative embodiment the cell is present in an individual. In a particular embodiment the cell is a cancer cell, for example a cell from a tumour cell line or a cell included in a tumour, including cancer cells from a tumour that can be treated by the inhibition of the activity of HDAC10.

Yet another aspect of the invention relates to the use of the compound as described above, or a prodrug thereof, for the preparation of a medicament for the treatment or prevention of a proliferative disorder or disease, including cancer, autoimmune disorders or neurodegeneration, in particular cancer, including cancers that can be treated by the inhibition of the activity of HDAC10. Additionally, the invention relates to a pharmaceutical composition comprising the compound as described above, or a prodrug thereof, and a pharmaceutically acceptable diluent, excipient or carrier, for the treatment of a proliferative disorder or disease, including cancer, autoimmune disorders or neurodegeneration, in particular cancer, including cancers that can be treated by the inhibition of the activity of a protein kinase or mutant thereof, such as the inhibition of the activity of HDAC10.

In another aspect, the present invention relates to the treatment of a patient after organ transplantation, and to a pharmaceutical composition comprising the compound as described above, or a prodrug thereof, and a pharmaceutically acceptable diluent, excipient or carrier, for use in the treatment of a patient who is about to receive, or has received, a foreign organ.

The subject compound is useful to treat various disorders or diseases, including proliferative disorders or diseases. The term “proliferative disorder or disease” is also art recognized and includes a disorder or disease affecting an individual, such as an animal, in a manner which is marked by aberrant, or otherwise unwanted, proliferation of a subset of cells of an individual. Cancer and tumors are proliferative disorders or diseases. Cells comprising or derived from a tumor will generally be understood to be a proliferating cell, typically a hyper-proliferating cell, and in other circumstances, a tumor cell may be dysplastic, or may have proliferated. In certain embodiments, said treatment is the treatment of a cancer that can be treated by the inhibition of the activity of a protein kinase or mutant thereof, such as the inhibition of the activity of HDAC10.

It will be apparent to a person skilled in the art, on reading the disclosure of the instant invention, that the methods, pharmaceutical compositions and packaged pharmaceuticals comprising the subject compound will be useful for the treatment of other proliferative disorders or diseases, or for killing or inhibiting proliferating cells including tumor cells.

the compound of the present invention may be useful in the treatment of disease processes which feature abnormal cellular proliferation, such as hyperproliferative diseases, including cancer, benign prostate hyperplasia, familial adenomatosis polyposis, neurofibromatosis, psoriasis, fungal infections, endotoxic shock, hypertrophic scar formation, inflammatory bowel disease, transplant rejection, vascular smooth muscle cell proliferation associated with atherosclerosis, psoriasis, pulmonary fibrosis, arthritis, glomerulonephritis, restenosis following angioplasty or vascular surgery, and other post-surgical stenosis and restenosis. See, for example, U.S. Pat. Nos. 6,114,365 and 6,107,305.

The compound disclosed herein is expected to be useful in the therapy of proliferative or hyperproliferative disorders or diseases such as cancer, autoimmune diseases, viral diseases, fungal diseases, neurodegenerative disorders and cardiovascular disease, or in the prevention of organ rejection after organ transplantation.

In certain embodiments, tumors may be solid tumors, which are cancer of body tissues other than blood, bone marrow, or the lymphatic system. In other embodiments, tumors may be hematological tumors, such as leukemia and lymphomas. Leukemia is a collective term for malignant diseases characterized by a proliferation of malignantly changed white blood cells. Diseases arising from lymphatic tissue are called lymphomas.

Solid tumors may be selected from: liver cancer, stomach cancer, colon cancer, breast cancer, pancreas cancer, prostate cancer, skin cancer, renal cancer, bone cancer, thyroid cancer, skin cancer, including squamous cell carcinoma, esophagus cancer, kidney cancer, bladder cancer, gall cancer, cervical cancer, ovarian cancer, lung cancer, bronchial, small and non-small-cell lung cancer, gastric, and head and neck cancer.

Hematological tumors may be leukemia, such as Acute Myelogenous Leukemia (AML), Acute Lymphoblastic Leukemia (ALL), Acute Lymphocytic Leukemia, Acute Leukemia, Acute Promyelocytic Leukemia, Chronic Granulocytic Leukemia (CGL), Chronic Leukemia, Chronic Lymphocytic Leukemia (CLL), Chronic Myelogenous Leukemia (CML), Chronic Myelomonocytic Leukemia, Common-type Acute Lymphoblastic Leukemia, Eosinophilic Leukemia, Erythroleukemia, Extranodal Lymphoma, Follicular Lymphoma, Hairy Cell Leukemia, Monocytic Leukemia, Prolymphocytic Leukemia.

Hematological tumors may also be lymphoma, such as B Cell Lymphomas, Burkitt Lymphoma, Cutaneous T Cell Lymphoma, High-Grade Lymphoma, Hodgkin's Lymphoma, Non-Hodgkin's Lymphoma, Low-grade Lymphoma, Lymphoblastic Lymphoma, Mantle Cell Lymphoma, Marginal Zone Lymphoma, Mucosa-Associated Lymphoid Tissue (MALT) Lymphomas, T Cell Lymphomas, peripheral T cell lymphoma, multiple myeloma, Essential Thrombocythemia, Hairy Cell Lymphoma, Extramedullary myeloma, Granulocytic Sarcomae.

Hematological tumors may also be tumors of myeloid lineage, including acute and chronic myelogenous leukemias, myelodysplastic syndrome, and promyelocytic leukaemia.

Tumors may also be of mesenchymal origin, such as fibrosarcoma and rhabdomyosarcoma. Furthermore, tumors may be tumors of the central and peripheral nervous system, such as astrocytoma, neuroblastoma, glioma, and schwannomas; and tumors may be other tumors, such as melanoma, seminoma, teratocarcinoma, osteosarcoma, xenoderoma pigmentosum, keratoctanthoma, thyroid follicular cancer, and Kaposi's sarcoma.

Tumors that are resistant or refractory to treatment with other anti-cancer or anti-proliferative agents may also benefit from treatment with the methods and pharmaceutical compositions of the present invention.

The compound disclosed herein may also be useful in the chemoprevention of cancer. Chemoprevention is defined as inhibiting the development of invasive cancer by either blocking the initiating mutagenic event or by blocking the progression of pre-malignant cells or inhibiting tumor relapse.

The compound disclosed herein may also be useful in inhibiting tumor angiogenesis and metastasis.

The compound of this invention may also be useful in combination (administered together or sequentially) with known anti-cancer treatments such as radiation therapy or with anti-cancer, anti-proliferative, cytostatic or cytotoxic agents. Other anti-cancer and anti-proliferative agents which may be used in combination with the compound of the present invention include those described herein. In combination treatment, the compound of the present invention may be further administered with any other anti-cancer and anti-proliferative agent disclosed herein.

If formulated as a fixed dose, such combination products employ the compound of this invention within the dosage range described herein and the other pharmaceutically active agent or treatment within its approved dosage range. For example, the cdc2 inhibitor olomucine has been found to act synergistically with known cytotoxic agents in inducing apoptosis (J. Cell Sci., 108, 2897 (1995)). The compound described herein may also be administered sequentially with known anti-cancer or anti-proliferative agents when a combination formulation is inappropriate. The invention is not limited in the sequence of administration; the compound described herein may be administered either prior to or after administration of the known anti-cancer or anti-proliferative agent. For example, the cytotoxic activity of the cyclin-dependent kinase inhibitor flavopiridol is affected by the sequence of administration with anticancer agents (Cancer Research, 57, 3375 (1997)).

### Further Aspects of the Invention

Another aspect the invention provides a pharmaceutical package, wherein said package includes the compound of the present invention. In certain embodiments, the package comprises instructions which indicate that said composition may be used for the treatment of an individual in need thereof, including a human. In certain other embodiments, the pharmaceutical package includes the compound of the present invention formulated together with another pharmaceutical ingredient such as an anti-cancer or anti-proliferative agent. In this case, the compound of the present invention and the other pharmaceutical ingredient may be formulated separately and in individual dosage amounts.

Other pharmaceutical ingredients that may be formulated together or separately with the compound of the present invention include but are not limited to other anti-cancer and anti-proliferative agents such as described above. In certain still further embodiments, the pharmaceutical package comprises instructions to treat a patient in need of such treatment. In yet another aspect the invention provides a pharmaceutical package for treating an individual suffering from a proliferative disorder or disease, such as a tumor or a cancer, wherein said package includes at least the compound of the present invention. In certain still further embodiments, the pharmaceutical package comprises instructions to treat the disorder.

As used herein the term “pharmaceutical package” or “pharmaceutical pack” refer to any packaging system for storing and dispensing individual doses of medication. Preferably the pharmaceutical package contains sufficient daily dosage units appropriate to the treatment period or in amounts which facilitate the patient's compliance with the regimen. In certain embodiments, the pharmaceutical pack comprises one or more vessels that include the active ingredient, e.g., the compound of the present invention. Such vessel can be a container such as a bottle, vial, syringe, or capsule, or may be a unit dosage form such as a pill. The active ingredient may be provided in the vessel in a pharmaceutically acceptable form or may be provided, for example, as a lyophilized powder. In further embodiments, the pharmaceutical pack may further include a solvent to prepare the active ingredient for administration. In certain embodiments, the active ingredient may be already provided in a delivery device, such as a syringe, or a suitable delivery device may be included in the pack. The pharmaceutical package may comprise pills, liquids, gels, tablets, dragees or the pharmaceutical preparation in any other suitable form. The package may contain any number of daily pharmaceutical dosage units. The package may be of any shape, and the unit dosage forms may be arranged in any pattern, such as circular, triangular, trapezoid, hexagonal or other patterns. One or more of the doses or subunits may be indicated, for example to aid the doctor, pharmacist or patient, by identifying such dose or subunits, such as by employing color-coding, labels, printing, embossing, scorings or patterns. The pharmaceutical package may also comprise instructions for the patient, the doctor, the pharmacist or any other related person.

Some embodiments comprise the administration of more than one active ingredient, including the compound as disclosed herein. Such administration may occur concurrently or sequentially. The active ingredients may be formulated together such that one administration delivers both components. Alternatively the active ingredients may be formulated separately. The pharmaceutical package may comprise the compound of the present invention and the other pharmaceutical ingredient in a single formulation, i.e., they are formulated together, or the compound of the present invention and the other pharmaceutical ingredient in individual formulations, i.e., they are formulated separately. Each formulation may comprise the compound of the present invention and the other pharmaceutical ingredient in individual dosage amounts (in approximately equal or unequal amounts). Administration of the compound of the present invention and the other pharmaceutical ingredient results in a concentration that results in a therapeutically effective amount of the combination.

As used herein, the term “instructions” means a product label and/or documents or other information describing relevant materials or methodologies pertaining to assembly, preparation or use of a kit or packaged pharmaceutical. These materials may include any combination of the following: background information, steps or procedures to follow, list of components, proposed dosages, warnings regarding possible side effects, instructions for administering the drug, technical support, and any other related documents. Instructions can be supplied in printed form, such as a package label or a package insert. Instructions for a packaged pharmaceutical or a pharmaceutical composition can be inserted in a delivery carton or finished package, e.g., as a package insert, and the text of such has been approved by a competent regulatory authority such as the Food and Drug Administration (FDA) of the United States. Alternatively or complementarily, instruction may also be stored in electronic form, e.g., on a computer-readable storage medium such as a computer-readable memory device, a centralized database, magnetic media such as hard disks, floppy disks, and magnetic tape; optical media such as compact discs, CD-ROMs and holographic devices; magneto-optical media such as floptical disks; and hardware devices that are specially configured to store and execute program code, such as application-specific integrated circuits (ASICs), programmable logic devices (PLDs) and ROM (read only memory) and RAM (random access memory) devices. Instructions may comprise a web address of an internet website from which more detailed instructions may be downloaded, or a recorded presentation. Instructions can contain one or multiple documents or future updates.

Another aspect of the invention provides the use of the compound of Formula (I) to inhibit activity of HDAC10. In another embodiment of this aspect, the compound of Formula (I) is used for the preparation of a composition for the inhibition of activity of HDAC10. In yet another embodiment, the invention provides methods to inhibit HDAC10.

Thus, in one aspect the invention relates to a pharmaceutical composition, including the compound of the present invention, and a pharmaceutically acceptable diluent, excipient or carrier.

In certain embodiments, such pharmaceutical composition comprises a therapeutically effective amount of said compound or prodrug.

In certain embodiments, such pharmaceutical composition is for the treatment of an individual in need thereof.

In certain embodiments of such pharmaceutical composition, said individual is a human.

In another aspect the invention relates to a pharmaceutical package, including a pharmaceutical composition of the present invention, and instructions which indicate that said pharmaceutical composition may be used for the treatment of an individual in need thereof.

In certain embodiments of such pharmaceutical package, said instructions indicate that said pharmaceutical composition may be used for the treatment of a human.

In certain embodiments of such pharmaceutical package, said instructions indicate that said pharmaceutical composition may be used for the treatment of an individual suffering from a proliferative disorder or disease.

In certain such embodiments, said individual is a human.

In another aspect the invention relates to a method for treating a proliferative disorder or disease in an individual, comprising administering a therapeutically effective amount of the compound or a pharmaceutical composition of the present invention.

In certain embodiments of such method, said individual is a mammal selected from: domestic mammal, cat, dog, horse, sheep, cow, rodent, and human.

In certain embodiments of such method, said mammal is a human.

In another aspect the invention relates to a method for treating a proliferative disorder or disease in an individual, comprising exposing cells included in said disorder or disease to the compound of the present invention.

In certain embodiments of such method, said compound, or a prodrug thereof, is administered to said individual.

In certain embodiments of such method, said individual is a mammal selected from: domestic mammal, cat, dog, horse, sheep, cow, rodent, and human.

In certain embodiments of such method, said mammal is a human.

In another aspect the invention relates to a method for inhibiting cell proliferation, comprising contacting a cell with the compound of the present invention.

In another aspect the invention relates to the compound of the present invention for the preparation of a medicament for the treatment of a proliferative disorder or disease.

In another aspect the invention relates to a pharmaceutical composition comprising the compound of the present invention and a pharmaceutically acceptable carrier, diluent or excipient, for the treatment of a proliferative disorder or disease.

In certain embodiments of a pharmaceutical package, method, use or pharmaceutical composition of the present invention, the proliferative disorder or disease is a cancer.

### EXAMPLES

### Experimental Section:

### General Considerations for Non-Piperidine Compounds:

Chemicals and solvents were purchased from commercial sources at the highest level of purity and used without purification. Anhydrous tetrahydrofuran was dispensed with an MBraun SPS800 Solvent Purification System. Thin layer chromatography (TLC) was carried out on glass silica plates (TLC Silica gel 60 F₂₅₄; Merck). TLC visualization was accomplished using 254 and 366 nm UV light or ninhydrin stain. High resolution mass spectrometry was recorded on a Bruker ApexQe FT-ICR instrument, (Department of Organic Chemistry, University of Heidelberg). NMR spectra were recorded on Bruker 400 MHz or 600 MHz instruments at 298.1 K.

### Analytical HPLC-Methods

Analytical HPLC/MS was performed on an Agilent 1260 Infinity system.

| method | | | |
|---|---|---|---|
| name | column | solvent system | gradient |
| Acidic | Kinetex 2.6 μm C18 | A: H₂O, | 1% B → 90% B |
| Method | 100 Å, LC column 50 × | 0.01% HCOOH; | over 6 min, then |
| | 2.1 mm; 40° C.; flow | B: MeCN, | 90% B to 99% B |
| | rate: 0.06 mL/min | 0.01% HCOOH | over 2 min |
| Basic | Gemini 5 μm C18 | A: H₂O, 0.1% | 1% B → 90% B |
| Method | 110 Å, LC column 50 × | NH₃; | over 6 min, then |
| | 2 mm; 40° C.; flow | B: MeCN | 90% B to 99% B |
| | rate: 0.06 mL/min | | over 2 min |

UV—detection at 254 nm

### Preparative HPLC Methods

Preparative HPLC was performed on an Agilent 1260 Infinity system.

| method name | column | solvent system |
|---|---|---|
| Acidic Method | Kinetex 5 μm C18 100 Å, | A: H₂O, 0.05% TFA; |
| | AXIA packed column 250 × | B: MeCN, 0.05% |
| | 21.2 mm; 20° C.; flow rate: | TFA |
| | 15.0 mL/min | |
| Basic Method | Gemini 5 μm C18 110 Å, | A: H₂O, 0.1% NH₃; |
| | AXIA packed column 250 × | B: MeCN |
| | 21.2 mm; 20° C.; flow rate: | |
| | 15.0 mL/min | |

UV—detection at 254 and 230 nm

Solvent was removed by lyophilization with a Christ alpha 2-4 LD plus freeze-dyer.

### Medium Pressure Column Chromatography (MPLC)

MPLC was performed in normal or reverse phase (RP) with a RediSep Rf system (Teledyne Isco) and RediSep Rf columns (Teledyne Isco). For RP purification, solvent A was water and B was MeCN. Normal phase separations used eluents as described.

### General Considerations for Piperidine Compounds:

Starting materials and reagents were purchased from different suppliers. No further purification was done. For R_{f}-determination thin-layer plates from Merck (TLC Silica gel 60 F₂₅₄ and TLC Silica gel 60 RP-18 F₂₅₄s) were used and analyzed under UV light (254 nm). Mass spectrometry (MS) was performed on Advion expression CMS spectrometer using an APCI ion source or ESI. Spectra for final compounds were recorded with high resolution mass spectrometry (HRMS) on Exactive device (Thermo Fisher Scientific) operating in ESI mode. Theoretical masses were calculated with Biological Magnetic Resonance Data Bank (www.bmrb.wisc.edu). ¹H NMR and ¹³C NMR spectra were recorded on Bruker Avance III HD spectrometer at 400 and 100 MHz by using the signal of the deuterated solvent as internal standard. Following abbreviation were used to report the spectra: ¹H: chemical shift δ (ppm), multiplicity (s=singlet, d=doublet, dd=doublet of doublets, t=triplet, q=quartet, m=multiplet, b=broad), integration, coupling constant (J in Hz). ¹³C, chemical shift δ (ppm). HMBC and HSQC experiments were applied for the assignment. The purity of the final compounds (>95%) was determined by HPLC and UV detection (λ=210 nm). HPLC analysis was performed using the following conditions: Eluent A, H₂O containing 0.05% TFA; Eluent B, acetonitrile containing 0.05% TFA, flow rate 1 mL/min, linear gradient conditions (0-4 min, A=90%, B=10%; 4-29 min, linear increase to 100% of B; 29-31 min, B=100%; 31-40 min, A=10%, B=90%), Phenomenex Kinetex 5 μm XB-C 18 (100 Å, 250×4.60 mm).

General Procedure A: Preparation of Hydroxamic Acids with Hydroxylamine

To a stirring solution of methyl ester (typically 50-200 mg, 1.0 equiv) in 1,4-dioxane (0.5-2 mL), KCN (0.6 equiv) was added and stirred at rt for 1 h, then an aqueous solution of hydroxylamine (50%, 0.5-2 mL, min. 30 equiv) was added. The solution was stirred at rt for up to 24 h, until TLC indicated complete conversion. The reaction mixture was concentrated in vacuo, and coevaporated with MeOH to dryness, then dissolved in a minimal volume of water/MeOH. Purification was carried out by preparative RP-HPLC or RP-MPLC.

General Procedure B: Amide Coupling with EDC

Amine 52.2HCl (typically 80-200 mg, 1.0 equiv), carboxylic acid (1.1 equiv), catalytic amounts of hydroxybenzotriazole (HOBt) for sterically demanding acids, and EDC (1.3 equiv) were suspended in CH₂Cl₂ (5-10 mL) and DIPEA (3.5 equiv) was added. The reaction mixture was stirred at rt for 12-48 h, until TLC indicated complete conversion of the amine, then concentrated in vacuo, redissolved in EtOAc (50 mL) and washed with sat. NaHCO₃ solution (2×40 mL), brine (40 mL), then dried (MgSO₄) and concentrated. The residue was used without further purification unless stated otherwise.

### General Procedure C: Reductive Amination and Esterification of Gamma-Amino Acids

Amino acid 42.HCl (100-150 mg, 1.0 equiv) and Na(OAc)₃BH (1.5 equiv) were suspended in dry THF (1.0 mL) under nitrogen atmosphere at 0° C. Aldehyde (10 equiv), NEt₃ (2.0 equiv) and further dry THF (1.0 mL) were added and the cooling bath was removed. The reaction mixture was stirred at rt for 2-3 h, then diluted with water (10 mL), basified with sat. NaHCO₃ solution until pH 12.0, then concentrated to dryness. The residue was redissolved in water (10 mL) and acidified with 25% HCl until pH 1.0, then the solvent was removed in vacuo. MeOH (25 mL) was added to the residue, stirred overnight, then concentrated, redissolved in sat. NaHCO₃ (30 mL) and extracted with CH₂Cl₂ (3×20 mL). The combined organic layers were dried (MgSO₄) and concentrated in vacuo.

General Procedure Da/b: alkylation of methyl piperidine-4-carboxylate

K₂CO₃ (2.5-3.75 equiv) was placed in a round-bottom flask and suspended in the indicated polar aprotic solvent ((a) DMF, (b) acetonitrile). Methyl piperidine-4-carboxylate (1.0 equiv) was added and stirred for 5 min. After adding the desired alkyl bromide electrophile (1.0-1.5 equiv), the mixture was stirred over night at rt. Product formation was observed by TLC analysis (EtOAc/cyclohexane, 66:33 (v/v)). When full conversion was reached, solvent was removed under reduced pressure. The crude residue was resuspended in water. Aqueous suspension was three times extracted with EtOAc. The combined organic layers were washed with saturated NaHCO₃-solution and NaCl-solution and dried over MgSO₄ before the solvent was evaporated under reduced pressure. Crude product was purified via flash column chromatography (EtOAc/cyclohexane).

General Procedure E: Ester Saponification with LiOH

The methyl ester was dissolved in THF and 1.5 equiv of LiOH solution (1 M in water) were added. The mixture was stirred for 4 h at 40° C. After removing solvent, the crude residue was resuspended in water and washed with EtOAc. The aqueous layer was evaporated and the product was used without further purification.

General Procedure F: amide coupling of O-tritylhydroxylamine with BOP-Cl

Carboxylate (1.0 equiv) and BOP-Cl (2.0 equiv) were placed in a round-bottom flask and suspended in CH₂Cl₂. After adding NEt₃ (4 equiv), the mixture was stirred for 10 min at rt. O-tritylhydroxylamine (1.2 equiv.) was added and stirred over night at rt. The reaction was quenched by removing solvent and resuspension in saturated NaHCO₃ solution. The Aqueous suspension was extracted with EtOAc (three times). Organic layer was washed with saturated NaHCO₃ solution and NaCl solution, dried over MgSO₄ before the solvent was evaporated under reduced pressure. Crude product was purified via flash column chromatography (CH₂Cl₂/MeOH).

General Procedure Ga/b: trityl deprotection of O-trityl hydroxamic Acids

Protected hydroxamic acid (1.0 equiv) was dissolved in dry CH₂Cl₂. TFA (10.0 equiv) and Et₃SiH (10.0 equiv) were added and the mixture was stirred at rt. Product formation was observed by TLC analysis (CH₂Cl₂/MeOH, 90:10 (v/v)). When full conversion was reached, the solvent was removed, and (a) the crude product was purified via flash column chromatography (H₂O/MeCN+0.5% TFA), or (b) the crude product was dissolved in water. The resulting aqueous solution was washed with cyclohexane three times and the aqueous layer was concentrated under reduced pressure. The product was used without further purification.

General Procedure H: Boc Deprotection with TFA

Boc protected compound was dissolved in CH₂Cl₂. TFA (10.0 equiv) and Et₃SiH (10.0 equiv) were added and the mixture was stirred for 2 h at 40° C. Solvent was removed under reduced pressure. Crude product was purified via flash column chromatography (H₂O/MeCN+0.5% TFA).

### General Procedure I: Alkylation of Nosyl Amides

Nosyl protected amine (1.0 equiv), alkyl bromide electrophile (2.0 equiv), K₂CO₃ (1.5 equiv) and KI (0.2 equiv) were suspended in DMF. Reaction mixture was stirred at 45° C. for 4 h and overnight at rt. Solvent was removed under reduced pressure. Residue was suspended in water/CH₂Cl₂. Aqueous layer was extracted with CH₂Cl₂ (three times). Organic layer was dried over MgSO₄ before the solvent was evaporated under reduced pressure. Crude product was purified via flash column chromatography (EtOAc/cyclohexane).

### General Procedure J: Nosyl Deprotection

Nosyl protected compound (1.0 equiv) and K₂CO₃ (4.0 equiv) were dissolved in acetonitrile. After adding thiophenol (3.0 equiv), the reaction mixture was stirred for 4 h at 35° C. Solvent was removed and crude product was purified via flash column chromatography (CH₂Cl₂/MeOH).

### Abbreviations

app apparent (NMR spectra)
BOP-Cl bis(2-oxo-3-oxazolidinyl)phosphinic chloride
DIPEA diisopropylethylamine
EDC 1-ethyl-3-(3-dimethylaminopropyl)carbodiimide.HCl
HATU O-(7-azabenzotriazol-1-yl)-N,N,N′,N′-tetramethyluronium-hexafluorphosphate
HOBt 1-hydroxybenzotriazole
Morpho CDI N-cyclohexyl-N′-(2-morpholinoethyl)carbodiimide methyl-p-toluenesulfonate
p pentet (NMR spectra)
q quartet (NMR spectra)
quant quantitative
rt room temperature, 22±2° C.

### Synthesis

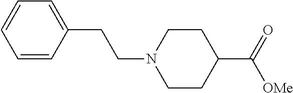 [SMILES: COC(=O)C1CCN(CCc2ccccc2)CC1]

Methyl 1-phenethylpiperidine-4-carboxylate (10): The title compound was prepared from methyl piperidine-4-carboxylate (859.1 mg, 6.00 mmol, 1.0 equiv) according to General Procedure Da to provide 10 as a colorless oil (1.0459 g, 4.23 mmol, 70% yield).

¹H NMR (400 MHz, DMSO-d₆) δ 7.30-7.24 (m, 2H, Phenyl H3,5), 7.23-7.14 (m, 3H, Phenyl H2,4,6), 3.60 (s, 3H, —O—CH₃), 2.88-2.85 (m, 2H, Piperidine H2,6), 2.73-2.69 (m, 2H, Phenyl-CH₂—CH₂), 2.49-2.41 (m, 2H, CH₂-CH₂—N), 2.31 (tt, J=11.2, 4.0 Hz, 1H, Piperidine H4), 2.01 (td, J=11.6, 2.4 Hz, 2H, Piperidine H2,6), 1.84-1.76 (m, 2H, Piperidine H3,5), 1.55 (dtd, J=13.2, 11.2, 3.6 Hz, 2H, Piperidine H3,5) ppm.

¹³C NMR (100 MHz, DMSO-d₆) δ 175.3 (—COOCH₃), 140.9 (Phenyl C1), 129.1 (Phenyl C2,6), 128.6 (Phenyl C3,5), 126.2 (Phenyl C4), 60.4 (CH₂-CH₂—N), 52.7 (Piperidine C2,6), 51.8 (—COOCH₃), 40.7 (Piperidine C4), 33.3 (Phenyl-CH₂—CH₂), 28.5 (Piperidine C3,5) ppm.

HR-MS (m/z): [M+H]⁺ calcd for C₁₅H₂₂NO₂⁺: 248.1645; found: 248.1644.

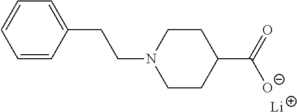 [SMILES: O=C([O-])C1CCN(CCc2ccccc2)CC1 ; [Li+]]

Lithium 1-phenethylpiperidine-4-carboxylate (11): The title compound was prepared from 10 (658.7 mg, 2.66 mmol, 1.0 equiv) according to General Procedure E to provide crude 11 as a white crystalline solid (1.0459 g, quant yield), which was used without further purification.

¹H NMR (400 MHz, DMSO-d₆) δ 7.30-7.23 (m, 2H, Phenyl H3,5), 7.23-7.12 (m, 3H, Phenyl H2,4,6), 2.86-2.79 (m, 2H, Piperidine H2,6), 2.74-2.67 (m, 2H, Phenyl-CH₂—CH₂), 2.45-2.40 (m, 2H, CH₂-CH₂—N), 1.94-1.85 (m, 2H, Piperidine H2,6), 1.79-1.65 (m, 3H, Piperidine H3,4,5), 1.53-1.40 (m, 2H, Piperidine H3,5) ppm.

¹³C NMR (100 MHz, DMSO-d₆) δ 179.0 (—COO⁻), 141.2 (Phenyl C1), 129.1 (Phenyl C2,6), 128.6 (Phenyl C3,5), 126.1 (Phenyl C4), 61.0 (CH₂-CH₂—N), 54.0 (Piperidine C2,6), 40.6 (Piperidine C4), 33.4 (Phenyl-CH₂—CH₂), 30.1 (Piperidine C3,5) ppm.

HR-MS (m/z): [M+H]⁺ calcd for C₁₄H₂₀NO₂⁺: 234.1489; found: 234.1488.

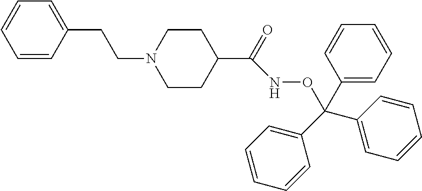 [SMILES: O=C(NOC(c1ccccc1)(c1ccccc1)c1ccccc1)C1CCN(CCc2ccccc2)CC1]

1-Phenethyl-N-(trityloxy)piperidine-4-carboxamide (12): The title compound was prepared from 11 (295.9 mg, 1.24 mmol, 1.0 equiv) according to General Procedure F to provide 12 as a white solid (354.5 mg, 0.723 mmol, 58% yield).

¹H NMR (400 MHz, DMSO-d₆) δ 10.31 (s, 1H, CO—NH—O), 7.37-7.28 (m, 15H, Trityl), 7.28-7.22 (m, 2H, Phenyl H3,5), 7.20-7.13 (m, 3H, Phenyl H2,4,6), 2.85-2.77 (m, 2H, Piperidine H 2,6), 2.69-2.62 (m, 2H, Phenyl-CH₂—CH₂), 2.44-2.36 (m, 2H, CH₂-CH₂—N), 1.97-1.84 (m, 1H, Piperidine H4), 1.82-1.68 (m, 2H, Piperidine H2,6), 1.31-1.18 (m, 4H, Piperidine H3,5) ppm.

¹³C NMR (100 MHz, DMSO-d₆) δ 172.93 (—CONH—), 142.86 (3× Phenyl C1 (Trityl)), 140.93 (Phenyl C1), 129.40 (Phenyl C2,6 or C3,5 (Trityl)), 129.03 (Phenyl C2,6), 128.58 (Phenyl C3,5), 127.89 (3× Phenyl C2,6 or C3,5 (Trityl)), 127.80 (3× Phenyl C4 (Trityl)), 126.18 (Phenyl C4), 92.24 (O—C-Trityl), 60.42 (CH₂-CH₂—N), 52.95 (Piperidine C2,6), 39.22 (Piperidine C4, only in HSQC), 33.18 (Phenyl-CH₂—CH₂), 28.46 (Piperidine C3,5) ppm.

HR-MS (m/z): [M+H]⁺ calcd for C₃₃H₃₅N₂O₂⁺: 491.2693; found: 491.2690.

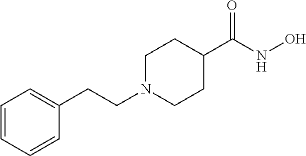 [SMILES: O=C(NO)C1CCN(CCc2ccccc2)CC1]

N-Hydroxy-1-phenethylpiperidine-4-carboxamide (DH22): The title compound was prepared from 12 (176.6 mg, 0.36 mmol, 1.0 equiv) according to General Procedure Ga to provide the TFA salt of DH22 as white solid (110.8 mg, 0.306 mmol, 85% yield).

¹H NMR (400 MHz, DMSO-d₆) δ 10.62 (s, 1H, CO—NH—OH), 9.49 (bs, 1H, CH₂—NH⁺-(Piperidine)), 8.85 (s, 1H, CO—NH—OH), 7.39-7.33 (m, 2H, Phenyl H3,5), 7.31-7.25 (m, 3H, Phenyl H2,4,6), 3.61 (d, J=12 Hz, 2H, Piperidine H2,6), 3.33-3.24 (m, 2H, CH₂—CH₂—N), 3.03-2.91 (m, 4H, Phenyl-CH₂—CH₂+Piperidine H 2,6), 2.32-2.23 (m, 1H, Piperidine H4), 1.94-1.77 (m, 4H, Piperidine H3,5) ppm.

¹³C NMR (100 MHz, DMSO-d₆) δ 170.15 (—CONH—), 158.53-158.23 (TFA), 137.33 (Phenyl C1), 129.11 (Phenyl C2,3,5,6), 127.29 (Phenyl C4), 57.09 (CH₂—CH₂—N), 51.49 (Piperidine C2,6), 36.87 (Piperidine C4), 29.94 (Phenyl-CH₂—CH₂), 26.24 (Piperidine C3,5) ppm.

HR-MS (m/z): [M+H]⁺ calcd for C₁₄H₂₁N₂O₂⁺: 249.1598; found: 249.1596.

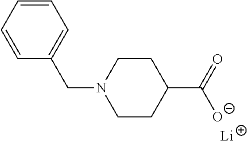 [SMILES: O=C([O-])C1CCN(Cc2ccccc2)CC1 ; [Li+]]

Lithium 1-benzylpiperidine-4-carboxylate (13): The title compound was prepared from methyl-1-benzylpiperidine-4-carboxylate (1015.8 mg, 4.35 mmol, 1.0 equiv) according to General Procedure E to provide 13 as an off-white crystalline solid (895.8 mg, 3.98 mmol, 92% yield).

¹H NMR (400 MHz, DMSO-d₆) δ 7.33-7.25 (m, 4H, Phenyl C2,3,5,6), 7.25-7.19 (m, 1H, Phenyl C4), 3.39 (s, 2H, Phenyl-CH₂—N), 2.75-2.66 (m, 2H, Piperidine H2,6), 1.87 (td, J=11.2, 2.4 Hz, 2H, Piperidine H2,6), 1.80 (tt, J=11.2, 3.6 Hz, 1H, Piperidine H3), 1.73-1.65 (m, 2H, Piperidine H3,5), 1.55-1.42 (m, 2H, Piperidine H3,5) ppm.

¹³C NMR (100 MHz, DMSO-d₆) δ (DMSO-d6, δ [ppm]): 179.30 (—COO⁻), 139.30 (Phenyl C1), 129.13 (Phenyl C2,6), 128.48 (Phenyl C3,5), 127.10 (Phenyl C4), 63.14 (Phenyl-CH₂—N), 53.91 (Piperidine C2,6), 44.10 (Piperidine C4), 29.86 (Piperidine C3,5) ppm.

HR-MS (m/z): [M−H]⁻ calcd for C₁₃H₁₆NO₂⁻: 218.1187; found: 218.1183.

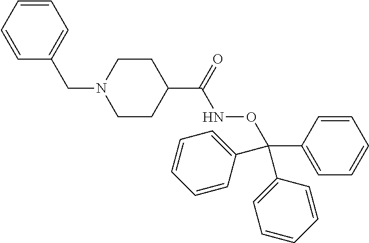 [SMILES: O=C(NOC(c1ccccc1)(c1ccccc1)c1ccccc1)C1CCN(Cc2ccccc2)CC1]

1-Benzyl-N-(trityloxy)piperidine-4-carboxamide (14): The title compound was prepared from 13 (420.7 mg, 1.87 mmol, 1.0 equiv) according to General Procedure F to provide 14 as a white solid (309.7 mg, 0.650 mmol, 35% yield).

¹H NMR (400 MHz, DMSO-d₆) δ 10.29 (s, 1H, CO—NH—O), 7.36-7.29 (m, 15H, Trityl), 7.28-7.26 (m, 2H, Phenyl H3,5), 7.25-7.19 (m, 3H, Phenyl H2,4,6), 3.34 (s, 2H, Phenyl-CH₂—N), 2.71-2.64 (m, 2H, Piperidine H 2,6), 1.96-1.84 (m, 1H, Piperidine H4), 1.80-1.65 (m, 2H, Piperidine H2,6), 1.32-1.16 (m, 4H, Piperidine H3,5) ppm.

¹³C NMR (100 MHz, DMSO-d₆) δ 172.89 (—CONH—), 142.84 (3× Phenyl C1 (Trityl)), 138.88 (Phenyl C1), 129.39 (Phenyl C2,6 or C3,5 (Trityl)), 129.08 (Phenyl C2,6), 128.52 (Phenyl C3,5), 127.89 (3× Phenyl C2,6 or C3,5 (Trityl)), 127.79 (3× Phenyl C4 (Trityl)), 127.21 (Phenyl C4), 92.23 (O—C-Trityl), 62.71 (Phenyl-CH₂—N), 52.87 (Piperidine C2,6), 39.29 (Piperidine C4, HSQC), 28.46 (Piperidine C3,5) ppm.

MS (APCI, +, m/z): [M+H]⁺ 477.2.

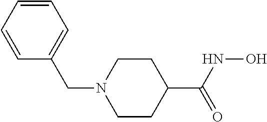 [SMILES: O=C(NO)C1CCN(Cc2ccccc2)CC1]

1-Benzyl-N-hydroxypiperidine-4-carboxamide (DH25): The title compound was prepared from 14 (143.6 mg, 0.30 mmol, 1.0 equiv) according to General Procedure Ga to provide the TFA salt of DH25 as a brown oil (101.8 mg, 0.292 mmol, 97% yield).

¹H NMR (400 MHz, DMSO-d₆) δ 10.60 (s, 1H, CO—NH—OH), 9.60 (bs, 1H, CH₂—NH⁺-Piperidine), 7.51-7.45 (m, 5H, Phenyl), 4.29 (d, J=4.8 Hz, 2H, Phenyl-CH₂—N), 3.37 (d, J=11.6 Hz, 2H, Piperidine H2,6), 3.05-2.87 (m, 2H, Piperidine H2,6), 2.29-2.18 (m, 1H, Piperidine H4), 1.94-1.67 (m, 4H, Piperidine H3,5) ppm.

¹³C NMR (100 MHz, DMSO-d₆) δ 170.11 (—CONH—), 158.47 (d, 32 Hz, TFA), 131.71 (Phenyl C2,6), 130.02 (Phenyl C3,5), 129.28 (Phenyl C4), 59.61 (Phenyl-CH₂—N), 51.11 (Piperidine C2,6), 36.83 (Piperidine C4), 26.01 (Piperidine C3,5) ppm.

HR-MS (m/z): [M+H]⁺ calcd for C₁₃H₁₉N₂O₂⁺: 235.1441; found: 235.1439.

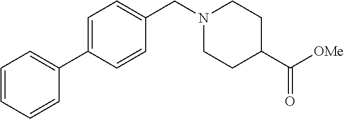 [SMILES: COC(=O)C1CCN(Cc2ccc(-c3ccccc3)cc2)CC1]

Methyl 1-([1,1′-biphenyl]-4-ylmethyl)-piperidine-4-carboxylate (15): The title compound was prepared from methyl piperidine-4-carboxylate (1287.8 mg, 9.00 mmol, 1.5 equiv) according to General Procedure Db to provide 15 as a white solid (1.771 g, 5.728 mmol, 96% yield).

¹H NMR (400 MHz, DMSO-d₆) δ 7.68-7.63 (m, 2H, Biphenyl H6,10), 7.63-7.59 (m, 2H, Biphenyl H3,11), 7.50-7.43 (m, 2H, Biphenyl H7,9), 7.40-7.33 (m, 3H, Biphenyl H2,8,12), 3.60 (s, 3H, —COOCH₃), 3.49 (s, 2H, Biphenyl-CH₂—N), 2.78 (d, J=11.4 Hz, 2H, Piperidine H2,6), 2.38-2.27 (tt, 11.2, 4.0 Hz, 1H, Piperidine H4), 2.06-1.96 (m, 2H, Piperidine, H2,6), 1.85-1.76 (m, 2H, Piperidine H3,5), 1.64-1.52 (m, 2H, Piperidine H3,5) ppm.

¹³C NMR (100 MHz, DMSO-d₆) δ 175.28 (—COOCH₃), 140.42 (Biphenyl C5), 139.20 (Biphenyl C4), 138.12 (Biphenyl C1), 129.77 (Biphenyl C2,12), 129.33 (Biphenyl C7,9), 127.71 (Biphenyl C8), 126.99 (Biphenyl C6,10), 126.90 (Biphenyl C3,11), 62.31 (Biphenyl-CH₂—N), 52.67 (Piperidine C2,6), 51.81 (—COOCH₃), 40.72 (Piperidine C4), 28.43 (Piperidine C3,5) ppm.

MS (APCI, +, m/z): [M+H]⁺ 310.2.

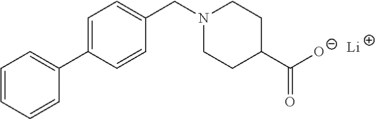 [SMILES: O=C([O-])C1CCN(Cc2ccc(-c3ccccc3)cc2)CC1 ; [Li+]]

Lithium 1-([1,1′-Biphenyl]-4-ylmethyl)-piperidine-4-carboxylate (16): The title compound was prepared from 15 (1002.2 mg, 3.24 mmol, 1.0 equiv) according to General Procedure E to provide 16 as a white crystalline solid (crude, quant yield), which was used without further purification.

¹H NMR (400 MHz, DMSO-d₆) δ 7.68-7.64 (m, 2H, Biphenyl H6,10), 7.63-7.58 (m, 2H, Biphenyl H3,11), 7.49-7.43 (m, 2H, Biphenyl H7,9), 7.39-7.33 (m, 3H, Biphenyl H2,8,12), 3.44 (s, 2H, Biphenyl-CH₂—N), 2.78-2.71 (m, 2H, Piperidine H2,6), 1.96-1.87 (m, 2H, Piperidine H2,6), 1.87-1.77 (m, 1H, Piperidine H4), 1.74-1.61 (m, 2H, Piperidine H3,5), 1.56-1.43 (m, 2H, Piperidine H3,5) ppm.

¹³C NMR (100 MHz, DMSO-d₆) δ 178.32 (—COO⁻), 140.49 (Biphenyl C5), 139.04 (Biphenyl C4), 138.61 (Biphenyl 01), 129.74 (Biphenyl C2,12), 129.33 (Biphenyl C7,9), 127.67 (Biphenyl C8), 126.98 (Biphenyl C6,10), 126.84 (Biphenyl C3,11), 62.74 (Biphenyl-CH₂—N), 53.92 (Piperidine C2,6), 43.97 (Piperidine C4), 29.86 (Piperidine C3,5) ppm.

HR-MS (m/z): [M+H]⁺ calcd for C₁₉H₂₂NO₂⁺: 296.1645; found: 296.1645.

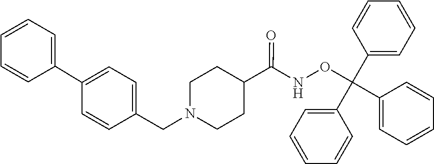 [SMILES: O=C(NOC(c1ccccc1)(c1ccccc1)c1ccccc1)C1CCN(Cc2ccc(-c3ccccc3)cc2)CC1]

1-([1,1′-Biphenyl]-4-ylmethyl)-N-(trityloxy)-piperidine-4-carboxamide (17): The title compound was prepared from crude 16 (493.7 mg, approx. 1.42 mmol, 1.0 equiv) according to General Procedure F (but using 4.0 equiv BOP-Cl) to provide 17 as a white solid (265.7 mg, 0.481 mmol, approx. 34% yield).

¹H NMR (400 MHz, DMSO-d₆) δ 10.31 (s, 1H, CO—NH—O), 7.67-7.63 (m, 2H, Biphenyl H6,10), 7.62-7.56 (m, 2H, Biphenyl H3,11), 7.49-7.43 (m, 2H, Biphenyl H7,9), 7.36-7.26 (m, 18H, Biphenyl H2,8,12+Trityl), 3.46-3.37 (m, 2H, Biphenyl-CH₂—N), 2.71-2.67 (m, 2H, Piperidine H2,6), 1.98-1.88 (m, 1H, Piperidine H4), 1.85-1.68 (s, 2H, Piperidine H2,6), 1.36-1.20 (s, 4H, Piperidine H3,5) ppm.

¹³C NMR (100 MHz, DMSO-d₆) δ 172.86 (—CONH—), 142.84 (3× Phenyl C1 (Trityl)+Biphenyl C2,12 (HMBC)), 140.41 (Biphenyl C5), 139.25 (Biphenyl C4 (HMBC)), 138.44 (Biphenyl C1 (HMBC)), 129.39 (3× Phenyl C2,6 or C3,5 (Trityl), 129.33 (Biphenyl C7,9), 127.90 (3× Phenyl C2,6 or C3,5 (Trityl)), 127.80 (3× Phenyl C4 (Trityl)), 127.72 (Biphenyl C8), 126.98 (Biphenyl C6,10), 126.87 (Biphenyl C3,11), 92.24 (O—C-Trityl), 62.30 (Biphenyl-CH₂—N), 52.88 (Piperidine C2,6), 39.35 (Piperidine C4 (HMBC)), 28.43 (Piperidine C3,5) ppm.

HR-MS (m/z): [M+H]⁺ calcd for C₃₈H₃₇N₂O₂⁺: 553.2850; found: 553.2850.

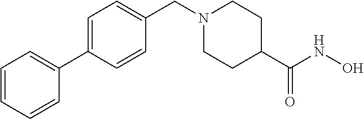 [SMILES: O=C(NO)C1CCN(Cc2ccc(-c3ccccc3)cc2)CC1]

1-([1,1′-Biphenyl]-4-ylmethyl)-N-hydroxypiperidine-4-carboxamide (DH35): The title compound was prepared from 17 (235.1 mg, 0.43 mmol, 1.0 equiv) according to General Procedure Ga to provide the TFA salt of DH35 as a brown oil (113.4 mg, 0.267 mmol, 62% yield).

¹H NMR (400 MHz, DMSO-d₆) δ 10.61 (s, 1H, CO—NH—OH), 9.60 (bs, 1H, CH₂—NH-Piperidine), 8.88 (s, 1H, CO—NH—OH), 7.82-7.76 (m, 2H, Biphenyl H3,11), 7.74-7.69 (m, 2H, Biphenyl H6,10), 7.65-7.56 (m, 2H, Biphenyl H2,12), 7.53-7.47 (m, 2H, Biphenyl H7,9), 7.44-7.38 (m, 1H, Biphenyl H8), 4.34 (d, J=4.4 Hz, 2H, Biphenyl-CH₂—N), 3.43 (d, J=11.6 Hz, 2H, Piperidine H2,6), 3.07-2.90 (m, 2H, Biphenyl-CH₂—N), 2.32-2.21 (m, 1H, Piperidine H4), 2.07-1.70 (m, 4H, Piperidine H3,5) ppm.

¹³C NMR (100 MHz, DMSO-d₆) δ 170.13 (—CONH—), 158.59-158.26 (TFA), 141.70 (Biphenyl C4), 139.65 (Biphenyl C4), 132.34 (Biphenyl C2,12), 129.48 (Biphenyl C7,9), 129.09 (Biphenyl C1), 128.37 (Biphenyl C8), 127.48 (Biphenyl C3,11), 127.21 (Biphenyl C6,10), 59.27 (Biphenyl-CH₂—N), 51.16 (Piperidine C2,6), 36.82 (Piperidine C4), 26.05 (Piperidine C3,5) ppm.

HR-MS (m/z): [M+H]⁺ calcd for C₁₉H₂₃N₂O₂⁺: 311.1754; found: 311.1755.

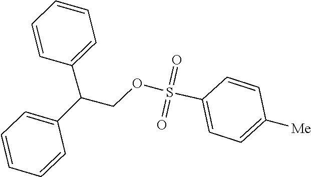 [SMILES: Cc1ccc(S(=O)(=O)OCC(c2ccccc2)c2ccccc2)cc1]

2,2-Diphenylethyl 4-methylbenzenesulfonate (18): 2,2-Diphenylethanol (800.0 mg, 4.04 mmol, 1.0 equiv) was dissolved in dry THF and cooled in ice-water. Under stirring, NaH (55-65% oil dispersion, 161.6 mg, 4.04 mmol, 1.0 equiv) was added in portion. p-Toluenesulfonyl chloride (997.5 mg, 5.25 mmol, 1.3 equiv) was added. The mixture was heated until the oil dispersion was melted and stirred under nitrogen over night at rt. Reaction was quenched by removing solvent and suspending the residue in a water-dichloromethane mixture. Aqueous layer was extracted with dichloromethane (three times). Collected organic layer was washed with saturated NaHCO₃ solution and NaCl solution and dried over MgSO₄ before the solvent was evaporated under reduced pressure. Crude product was purified via flash column chromatography (EtOAc/cyclohexane) to provide 18 as white flakes (405.5 mg, 1.150 mmol, 28% yield).

¹H NMR (400 MHz, DMSO-d₆) δ 7.71-7.68 (m, 2H, Tosyl H2,6), 7.46-7.42 (m, 2H, Tosyl H2,6), 7.30-7.18 (m, 10H, Diphenyl), 4.57 (d, J=7.8 Hz, 2H, CH—CH₂—O), 4.35 (t, J=7.8 Hz, 1H, Diphenyl-CH—CH₂), 2.43 (s, 3H, Tosyl-CH₃) ppm.

¹³C NMR (100 MHz, DMSO-d₆) δ 145.41 (Tosyl C1), 140.63 (Tosyl C4), 132.48 (2× Phenyl C1), 130.57 (Tosyl C3,5), 128.97 (2× Phenyl C3,5), 128.27 (2× Phenyl C2,6), 128.03 (Tosyl C2,6), 127.28 (2× Phenyl C4), 72.24 (CH-CH₂—O), 49.74 (Diphenyl-CH—CH₂), 21.54 (Tosyl-CH₃) ppm.

HR-MS (m/z): [M+Na]⁺ calcd for C₂₁H₂₀NaO₃S⁺: 375.1025; found: 375.1025.

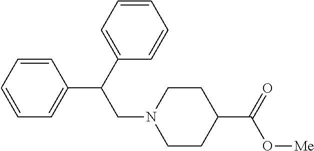 [SMILES: COC(=O)C1CCN(CC(c2ccccc2)c2ccccc2)CC1]

Methyl 1-(2,2-diphenylethyl)-piperidine-4-carboxylate (19): 18 (384.2 mg, 1.09 mmol, 1 equiv) was placed in a dry round-bottom flask and suspended in dry MeCN. Methyl piperidine-4-carboxylate (234.7 mg, 1.64 mmol, 1.5 equiv) and NaI (1.21 equiv) were added. After adding NEt₃ (220.6 mg, 2.18 mmol, 2.0 equiv) in one portion, the mixture was stirred at rt. Product formation was observed by TLC analysis (EtOAc/cyclohexane). Because of insufficient product formation silver carbonate (2.0 equiv) was added and the mixture was stirred for 2 h under reflux at 90° C. Solvent was evaporated. Crude residue was suspended in water and extracted with EtOAc. Organic layer was dried over MgSO₄ before the solvent was evaporated under reduced pressure. Crude product was purified by flash column chromatography (EtOAc/cyclohexane) to provide 19 as a white solid (109.8 mg, 0.340 mmol, 35% yield).

¹H NMR (400 MHz, DMSO-d₆) δ 7.33-7.23 (m, 8H, 2× Diphenyl H2,3,5,6), 7.18-7.12 (m, 2H, 2× Diphenyl H3), 4.24 (t, J=8.0 Hz, 1H, Diphenyl-CH—CH₂), 3.57 (s, 3H—COOCH₃), 2.92-2.83 (m, 4H, —CH—CH₂-Piperidine+Piperidine H2,6), 2.25 (tt, J=11.2, 4.0 Hz 1H, Piperidine H4), 2.01 (td, J=11.4, 2.2 Hz, 2H, Piperidine H2,6), 1.76-1.67 (m, 2H, Piperidine H3,5), 1.46-1.34 (m, 2H, Piperidine H3,5) ppm.

¹³C NMR (100 MHz, DMSO-d₆) δ 175.27 (—COOCH₃), 144.59 (2× Diphenyl C1), 128.61 (2× Diphenyl C3,5), 128.35 (2× Diphenyl C2,6), 126.37 (2× Diphenyl C4), 63.23 (—CH—CH₂—N), 52.92 (Piperidine C2,6), 51.77 (COOCH₃), 48.33 (Diphenyl-CH—CH₂), 28.32 (Piperidine C3,5) ppm.

Signal of Piperidine C1 not visible.

HR-MS (m/z): [M+H]⁺ calcd for C₂₁H₂₆NO₂⁺: 324.1958; found: 324.1959.

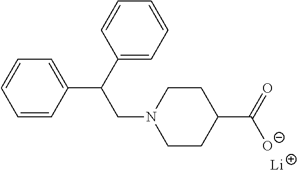 [SMILES: O=C([O-])C1CCN(CC(c2ccccc2)c2ccccc2)CC1 ; [Li+]]

Lithium 1-(2,2-Diphenylethyl)-piperidine-4-carboxylate (20): The title compound was prepared from 19 (112.2 mg, 0.35 mmol, 1.0 equiv) according to General Procedure E to provide 20 as a white crystalline solid (crude, quant yield), which was used without further purification.

¹H NMR (400 MHz, DMSO-d₆) δ 7.31-7.21 (m, 8H, 2× Diphenyl H2,3,5,6), 7.17-7.12 (m, 2H, 2× Diphenyl H3), 4.24 (t, J=7.6 Hz, 1H, Diphenyl-CH—CH₂), 2.88-2.80 (m, 4H, CH—CH₂—Piperidine+Piperidine H2,6), 1.94-1.86 (m, 2H, Piperidine H2,6), 1.80-1.69 (m, 1H, Piperidine H4), 1.66 (s, 4H), 1.67-1.58 (m, 2H, Piperidine H3,5), 1.41-1.28 (m, 2H, Piperidine H3,5) ppm.

¹³C NMR (100 MHz, DMSO-d₆) δ 179.23 (COO⁻), 144.80 (2× Diphenyl C1), 128.58 (2× Diphenyl C3,5), 128.37 (2× Diphenyl C2,6), 126.30 (2× Diphenyl C4) 63.81 (CH-CH₂—N), 54.23 (Piperidine C2,6), 48.38 (Diphenyl-CH—CH₂), 44.15 (Piperidine C4), 29.83 (Piperidine C3,5) ppm.

HR-MS (m/z): [M+H]⁺ calcd for C₂₀H₂₄NO₂⁺: 310.1802; found: 310.1802.

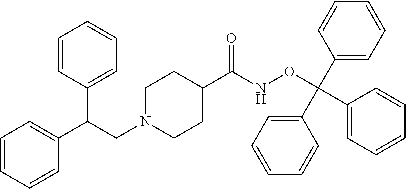 [SMILES: O=C(NOC(c1ccccc1)(c1ccccc1)c1ccccc1)C1CCN(CC(c2ccccc2)c2ccccc2)CC1]

1-(2,2-Diphenylethyl)-N-(trityloxy)-piperidine-4-carboxamide (21): The title compound was prepared from crude 20 (119.1 mg, 0.35 mmol, 1.0 equiv) according to General Procedure F to provide 21 as a white solid (46.0 mg, 0.081 mmol, 23% yield).

¹H NMR (400 MHz, DMSO-d₆) δ 10.26 (s, 1H, —CONHO—), 7.40-7.27 (m, 15H, Trityl), 7.26-7.20 (m, 8H, 2× Diphenyl H2,3,5,6), 7.17-7.10 (m, 2H, 2× Diphenyl H4), 4.19 (t, J=7.6 Hz, 1H, Diphenyl-CH—CH₂), 2.85-2.77 (m, 4H, CH—CH₂—Piperidine+Piperidine H2,6), 1.90-1.80 (m, 1H, Piperidine H4), 1.80-1.69 (m, 2H, Piperidine H2,6), 1.22-1.02 (m, 4H, Piperidine H3,5) ppm.

¹³C NMR (100 MHz, DMSO-d₆) δ 172.90 (—CONH—), 144.59 (2× Diphenyl C1), 142.82 (3× Phenyl C1 (Trityl)), 129.37 (Phenyl C2,6 or C3,5 (Trityl)), 128.58 (2× Diphenyl H3,5), 128.33 (2× Diphenyl H2,6), 127.88 (Phenyl C2,6 or C3,5 (Trityl)), 127.78 (3× Phenyl C4 (Trityl)), 126.34 (2× Diphenyl C4), 92.21 (O—C-Trityl), 63.30 (CH-CH₂—N), 53.15 (Piperidine C2,6), 48.18 (Diphenyl-CH—CH₂), 28.36 (Piperidine C3,5) ppm.

Piperidine C4 not visible.

HR-MS (m/z): [M+H]⁺ calcd for C₃₉H₃₉N₂O₂⁺: 567.3006; found: 567.3005.

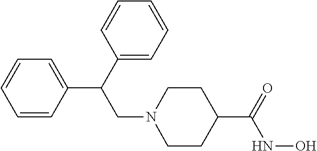 [SMILES: O=C(NO)C1CCN(CC(c2ccccc2)c2ccccc2)CC1]

1-(2,2-Diphenylethyl)-N-hydroxypiperidine-4-carboxamide (DH40): The title compound was prepared from 21 (43.0 mg 0.076 mmol, 1.0 equiv) according to General Procedure Ga to provide the TFA salt of DH40 as a brown oil (35.7 mg, 0.072 mmol, 94% yield).

¹H NMR (400 MHz, DMSO-d₆) δ 10.57 (s, 1H, CO—NH—OH), 9.07 (bs, 1H, CH₂—NH⁺-(Piperidine)), 7.50-7.41 (m, 4H, 2× Diphenyl H2,6), 7.39-7.32 (m, 4H, 2× Diphenyl H3,5), 7.29-7.22 (m, 2H, 2× Diphenyl H4), 4.59 (t, J=7.6 Hz, 1H, Diphenyl-CH—CH₂), 3.94-3.88 (m, 2H, CH—CH₂—Piperidine), 3.58-3.48 (m, 2H, Piperidine H2,6), 3.04-2.90 (m, 2H, Piperidine H2,6), 2.26-2.15 (m, 1H, Piperidine H4), 1.91-1.72 (m, 4H, Piperidine H3,5) ppm.

¹³C NMR (100 MHz, DMSO-d₆) δ 141.96 (2× Diphenyl 01), 129.29 (Diphenyl H3,5), 128.07 (Diphenyl H2,6), 127.54 (Diphenyl C4), 59.98 (CH-CH₂—N), 52.26 (Piperidine C2,6), 45.78 (Piperidine C2,6), 36.60 (Piperidine C4), 25.75 (Piperidine C3,5) ppm

CONHO not visible.

HR-MS (m/z): [M+H]⁺ calcd for C₂₀H₂₅N₂O₂⁺: 325.1911; found: 325.1913.

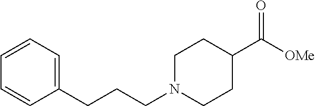 [SMILES: COC(=O)C1CCN(CCCc2ccccc2)CC1]

Methyl 1-(3-phenylpropyl)-piperidine-4-carboxylate (22): The title compound was prepared from methyl piperidine-4-carboxylate (859.1 mg, 6.00 mmol, 1.0 equiv) according to General Procedure Db to provide 22 as a colorless oil (561.2 mg, 3.279 mmol, 55% yield).

¹H NMR (400 MHz, DMSO-d₆) δ 7.30-7.24 (m, 2H, Phenyl H3,5), 7.22-7.14 (m, 3H, Phenyl H2,4,6), 3.60 (s, 3H, —O—CH₃), 2.81-2.73 (m, 2H, Piperidine H2,6), 2.60-2.54 (m, 2H, Phenyl-CH₂—CH₂), 2.35-2.21 (m, 3H, CH₂-CH₂—N+Piperidine H4), 1.97-1.87 (m, 2H, Piperidine H2,6), 1.83-1.75 (m, 2H, Piperidine H3,5), 1.75-1.65 (m, 2H, CH₂—CH₂—N), 1.61-1.49 (m, 2H, Piperidine H3,5) ppm.

¹³C NMR (100 MHz, DMSO-d₆) δ 175.31 (—COOCH₃), 142.49 (Phenyl C1), 128.71 (Phenyl C2,6), 128.63 (Phenyl C3,5), 126.04 (Phenyl C4), 57.73 (CH₂-CH₂—N), 52.77 (Piperidine C2,6), 51.79 (—COOCH₃), 40.90 (Piperidine C4), 33.32 (Phenyl-CH₂—CH₂), 28.67 (CH₂—CH₂—N), 28.44 (Piperidine C3,5) ppm.

MS (APCI, +, m/z): [M+H]⁺ 262.3.

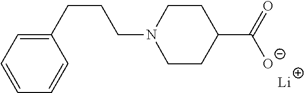 [SMILES: O=C([O-])C1CCN(CCCc2ccccc2)CC1 ; [Li+]]

Lithium 1-(3-phenylpropyl)-piperidine-4-carboxylate (23): The title compound was prepared from 22 (431.6 mg, 1.65 mmol, 1.0 equiv) according to General Procedure E to provide 23 as a white crystalline solid (crude, quant yield).

¹H NMR (400 MHz, DMSO-d₆) δ 7.30-7.23 (m, 2H, Phenyl H3,5), 7.22-7.13 (m, 3H, Phenyl H2,4,6), 2.79-2.71 (m, 2H, Piperidine H2,6), 2.60-2.53 (m, 2H, Phenyl-CH₂—CH₂), 2.25-2.18 (m, 2H, CH₂-CH₂—N), 1.96-1.87 (m, 1H, Piperidine H4), 1.87-1.77 (m, 2H, Piperidine H2,6), 1.74-1.64 (m, 4H, Piperidine H3,5+Phenyl-CH₂—CH₂), 1.55-1.43 (m, 2H, Piperidine H3,5) ppm.

¹³C NMR (100 MHz, DMSO-d₆) δ 178.60 (—COO⁻), 142.57 (Phenyl C1), 128.71 (Phenyl C2,6), 128.62 (Phenyl C3,5), 126.01 (Phenyl C4), 58.00 (CH₂-CH₂—N), 53.69 (Piperidine C2,6), 43.25 (Piperidine C4), 33.39 (Phenyl-CH₂—CH₂), 29.49 (Piperidine C3,5), 28.78 (Phenyl-CH₂—CH₂) ppm.

MS (APCI, +, m/z): [M+H]⁺ 248.2.

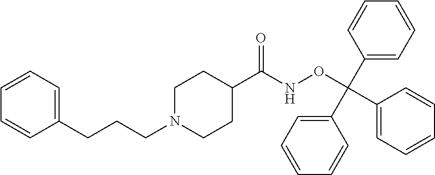 [SMILES: O=C(NOC(c1ccccc1)(c1ccccc1)c1ccccc1)C1CCN(CCCc2ccccc2)CC1]

1-(3-Phenylpropyl)-N-(trityloxy)-piperidine-4-carboxamide (24): The title compound was prepared from 23 (236.4 mg, 0.96 mmol, 1.0 equiv) according to General Procedure F to provide 24 as a white solid (204.0 mg, 0.405 mmol, 42% yield).

¹H NMR (400 MHz, DMSO-d₆) δ 10.29 (s, 1H, —CONHO—), 7.40-7.30 (m, 15H, Trityl), 7.30-7.22 (m, 2H, Phenyl H3,5), 7.19-7.13 (m, 3H, Phenyl H2,4,6), 2.80-2.63 (m, 2H, Piperidine H2,6), 2.57-2.52 (m, 2H, Phenyl-CH₂—CH₂), 2.23-2.07 (m, 2H, CH₂-CH₂—N), 1.95-1.83 (m, 1H, Piperidine H4), 1.73-1.57 (m, 4H, Piperidine H2,6+Phenyl-CH₂-CH₂), 1.33-1.17 (m, 4H, Piperidine H3,5) ppm.

¹³C NMR (100 MHz, DMSO-d₆) δ 172.95 (—CONH—), 142.85 (3× Phenyl C1 (Trityl)), 142.45 (Phenyl C1), 129.39 (Phenyl C2,6 or C3,5 (Trityl)), 128.70 (Phenyl C2,6), 128.62 (Phenyl C3,5), 127.89 (3× Phenyl C2,6 or C3,5 (Trityl)), 127.79 (3× Phenyl C4 (Trityl)), 126.03 (Phenyl C4), 92.23 (O—C-Trityl), 57.73 (CH₂-CH₂—N), 53.04 (Piperidine C2,6), 39.65 (Piperidine C4 (HSQC)) 33.27 (Phenyl-CH₂—CH₂), 28.63 (Phenyl-CH₂—CH₂), 28.47 (Piperidine C3,5) ppm.

MS (APCI, +, m/z): [M+H]⁺ 505.2.

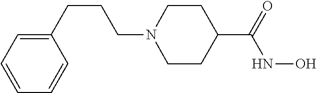 [SMILES: O=C(NO)C1CCN(CCCc2ccccc2)CC1]

N-Hydroxy-1-(3-phenylpropyl)-piperidine-4-carboxamide (DH53): The title compound was prepared from 24 (94.5 mg, 0.19 mmol, 1.0 equiv) according to General Procedure Ga to provide the TFA salt of DH53 as a colorless oil (71.4 mg, 0.190 mmol, quant yield).

¹H NMR (400 MHz, DMSO-d₆) δ 10.60 (s, 1H, CO—NH—OH), 10.14 (s, 1H, CH₂—NH⁺-(Piperidine)), 9.16 (s, 1H, CO—NH—OH), 7.35-7.28 (m, 2H, Phenyl H3,5), 7.27-7.19 (m, 3H, Phenyl H2,4,6), 3.52 (d, J=11.6 Hz, 2H, Piperidine H2,6), 3.08-3.01 (m, 2H, CH₂—CH₂—N), 2.99-2.84 (m, 2H, Piperidine H2,6), 2.63 (t, J=7.6 Hz, 2H, Phenyl-CH₂—CH₂), 2.30-2.19 (m, 1H, Piperidine H4), 2.01-1.89 (m, 2H, CH₂—CH₂—CH₂), 1.88-1.72 (m, 4H, Piperidine H3,5) ppm.

¹³C NMR (100 MHz, DMSO-d₆) δ 170.14 (—CONH—), 159.06+158.72+159.38+158.04 (TFA), 140.84 (Phenyl C1), 128.88 (Phenyl C3,5), 128.67 (Phenyl C2,6), 126.60 (Phenyl C4), 56.05 (CH₂—CH₂—N), 51.43 (Piperidine C2,6), 36.75 (Piperidine C4), 32.37 (Phenyl-CH₂—CH₂), 26.25 (Piperidine C3,5), 25.41 (CH₂—CH₂—CH₂) ppm.

HR-MS (m/z): [M+H]⁺ calcd for C₁₅H₂₃N₂O₂⁺: 263.1754; found: 263.1756.

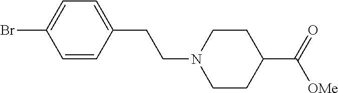 [SMILES: COC(=O)C1CCN(CCc2ccc(Br)cc2)CC1]

Methyl 1-(4-bromophenethyl)-piperidine-4-carboxylate (25): The title compound was prepared from methyl piperidine-4-carboxylate (519.8 mg, 3.63 mmol, 1.0 equiv) according to General Procedure Db to provide 25 as a yellow/white crystalline solid (912.2 mg, 2.806 mmol, 77% yield).

¹H NMR (400 MHz, DMSO-d₆) δ 7.47-7.43 (m, 2H, Phenyl H3,5), 7.21-7.17 (m, 2H, Phenyl H2,6), 3.60 (s, 3H, —O—CH₃), 2.88-2.80 (m, 2H, Piperidine H2,6), 2.72-2.65 (m, 2H, Phenyl-CH₂—CH₂), 2.48-2.43 (m, 2H, CH₂—CH₂—N), 2.35-2.25 (m, 1H, Piperidine H4), 2.05-1.95 (m, 2H, Piperidine H2,6), 1.83-1.75 (m, 2H, Piperidine H3,5), 1.59-1.47 (m, 2H, Piperidine H3,5) ppm.

¹³C NMR (100 MHz, DMSO-d₆) δ 175.29 (—\vec{C}OOCH₃), 140.47 (Phenyl C1), 131.41 (Phenyl C3,5), 131.37 (Phenyl C2,6), 119.24 (Phenyl C4), 59.93 (CH₂-CH₂—N), 52.65 (Piperidine C2,6), 51.80 (—CH₃), 40.82 (Piperidine C4 (HSQC)), 32.45 (Phenyl-CH₂—CH₂), 28.42 (Piperidine C3,5) ppm.

MS (APCI, +, m/z): [M+H]⁺ 326.0.

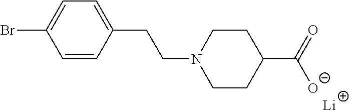 [SMILES: O=C([O-])C1CCN(CCc2ccc(Br)cc2)CC1 ; [Li+]]

Lithium 1-(4-Bromophenethyl)-piperidine-4-carboxylate (26): The title compound was prepared from 25 (810.2 mg, 2.49 mmol, 1.0 equiv) according to General Procedure E to provide 26 as a white cristals (699.8 g, 2.20 mmol, 88% yield as Li salt).

¹H NMR (DMSO-d6, δ [ppm]): 7.47-7.42 (m, 2H, Phenyl H3,5), 7.21-7.16 (m, 2H, Phenyl H2,6), 2.85-2.78 (m, 2H, Piperidine H2,6), 2.71-2.65 (m, 2H, Phenyl-CH₂—CH₂), 2.46-2.39 (m, 2H, CH₂—CH₂—N), 1.96-1.83 (m, 3H, Piperidine H2,6+H4), 1.74-1.66 (m, 2H, Piperidine H3,5), 1.53-1.40 (m, 2H, Piperidine H3,5).

¹³C NMR (DMSO-d6, δ [ppm]): 177.92 (—COO⁻), 140.66 (Phenyl 01), 131.38 (Phenyl C3,5+C2,6), 119.17 (Phenyl C4), 60.31 (CH₂-CH₂—N), 53.65 (Piperidine C2,6), 43.37 (Piperidine C4), 32.56 (Phenyl-CH₂—CH₂), 29.57 (Piperidine C3,5).

MS (APCI, +, m/z): [M+H]⁺ 311.9+313.9

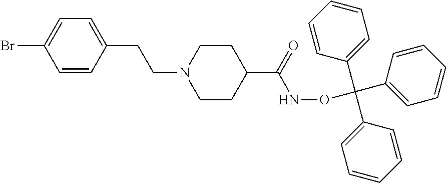 [SMILES: O=C(NOC(c1ccccc1)(c1ccccc1)c1ccccc1)C1CCN(CCc2ccc(Br)cc2)CC1]

1-(4-Bromophenethyl)-N-(trityloxy)-piperidine-4-carboxamide (27): The title compound was prepared from 26 (502.6 mg, 1.62 mmol, 1.0 equiv) according to General Procedure F to provide 27 as a white solid (526.3 mg, 0.924 mmol, 57% yield).

¹H NMR (DMSO-d6, δ [ppm]): 10.30 (s, 1H, CONH—O), 7.46-7.41 (m, 2H, Phenyl H3,5), 7.36-7.27 (m, 15H, Trityl), 7.17-7.13 (m, 2H, Phenyl H2,6), 2.84-2.75 (m, 2H, Piperidine H2,6), 2.67-2.60 (m, 2H, Phenyl-CH₂—CH₂), 2.43-2.35 (m, 2H, CH₂—CH₂—N), 1.95-1.85 (s, 1H, Piperidine H4), 1.82-1.67 (s, 2H, Piperidine H2,6), 1.33-1.15 (m, 4H, Piperidine H3,5).

¹³C NMR (DMSO-d6, δ [ppm]): 172.90 (—CONH—), 142.85 (3× Phenyl C1 (Trityl)), 140.48 (Phenyl C1), 131.38 (Phenyl C3,5), 131.35 (Phenyl C2,6), 129.40 (Phenyl C2,3,5,6 (Trityl)), 127.90 (Phenyl C2,3,5,6 (Trityl)), 127.80 (3× Phenyl C4 (Trityl)), 119.22 (Phenyl C4), 92.24 (O—C-Trityl), 59.91 (CH₂—CH₂—N), 52.90 (Piperidine C2,6), 39.19 (Piperidine C4, (HSQC)), 32.38 (Phenyl-CH₂—CH₂), 28.42 (Piperidine C3,5).

MS (APCI, +, m/z): [M+H]⁺ 568.8+570.9

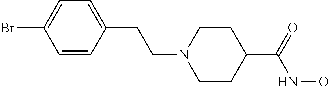 [SMILES: O=C(NO)C1CCN(CCc2ccc(Br)cc2)CC1]

1-(4-Bromophenethyl)-N-hydroxypiperidine-4-carboxamide (DH67): The title compound was prepared from 27 (281.1 mg, 0.494 mmol, 1.0 equiv) according to General Procedure Ga to provide TFA salt of DH67 as a white/brown solid (201.7 mg, 0.457 mmol, 93% yield as TFA salt).

¹H NMR (DMSO-d6, δ [ppm]): 10.61 (s, 1H, CO—NH—OH), 9.40 (s, 1H, CH₂—NH⁺-(Piperidine)), 7.59-7.53 (m, 2H, Phenyl H3,5), 7.30-7.23 (m, 2H, Phenyl H2,6), 3.65-3.54 (m, 2H, Piperidine, H2,6), 3.34-3.23 (m, 2H, CH₂—CH₂—N), 3.03-2.88 (m, 4H, Phenyl-CH₂—CH₂+Piperidine H 2,6), 2.32-2.22 (m, 1H, Piperidine H4), 1.94-1.73 (m, 4H, Piperidine H3,5).

¹³C NMR (DMSO-d6, δ [ppm]): 170.12 (—CONH—), 258.75+158.40 (TFA), 136.77 (Phenyl C1), 131.96 (Phenyl C3,5), 131.43 (Phenyl C2,6), 120.44 (Phenyl C4), 56.70 (CH₂-CH₂—N), 51.52 (Piperidine C2,6), 36.81 (Piperidine C4), 29.24 (Phenyl-CH₂—CH₂), 26.19 (Piperidine C3,5).

HR-MS (ESI, m/z): [M+H]⁺ calcd for C₁₄H₂₀BrN₂O₂⁺: 327.0708+329.0801; found: 327.0701+329.0680.

Purity (HPLC, λ=210 nm): 99.5% (t_{R}=12.78 min)

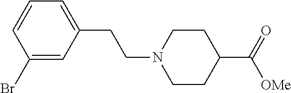 [SMILES: COC(=O)C1CCN(CCc2cccc(Br)c2)CC1]

Methyl 1-(3-bromophenethyl)-piperidine-4-carboxylate (28): The title compound was prepared from methyl piperidine-4-carboxylate (780.4 mg, 5.45 mmol, 1.5 equiv) according to General Procedure Db (modifications 1.5 equiv methyl piperidine-4-carboxylate, 3.0 equiv K₂CO₃) to provide 28 as a colorless crystalline solid (827.9 mg, 2.54 mmol, 70% yield).

¹H NMR (DMSO-d6, δ [ppm]): 7.46-7.43 (m, 1H, Phenyl H2), 7.41-7.35 (m, 1H, Phenyl H4), 7.26-7.22 (m, 2H, Phenyl H5,6), 3.60 (s, 3H, O—CH₃), 2.89-2.81 (m, 2H, Piperidine H2,6), 2.75-2.69 (m, 2H, Phenyl-CH₂—CH₂), 2.49-2.43 (partially covert by DMSO) (m, 2H, CH₂-CH₂—N), 2.35-2.26 (m, 1H, Piperidine H4), 2.06-1.95 (m, 2H, Piperidine H2,6), 1.84-1.75 (m, 2H, Piperidine H3,5), 1.60-1.46 (m, 2H, Piperidine H3,5).

¹³C NMR (DMSO-d6, δ [ppm]): 175.29 (—COOH₃), 143.96 (Phenyl C1), 131.82 (Phenyl C2), 130.73 (Phenyl C4), 129.09 (Phenyl C6), 128.25 (Phenyl C5), 121.91 (Phenyl C3), 59.88 (CH₂-CH₂—N), 52.62 (Piperidine C2,6), 51.80 (—COOCH₃), 40.60 (Piperidine C4 (HMBC)), 32.62 (Phenyl-CH₂—CH₂), 28.42 (Piperidine C3,5).

MS (APCI, +, m/z): [M+H]⁺ 325.9+327.9

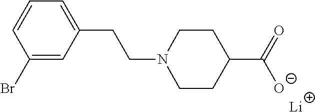 [SMILES: O=C([O-])C1CCN(CCc2cccc(Br)c2)CC1 ; [Li+]]

Lithium 1-(3-bromophenethyl)-piperidine-4-carboxylate (29): The title compound was prepared from 28 (737.3 mg, 2.27 mmol, 1.0 equiv) according to General Procedure E to provide 29 as a white crystalline solid (539.5 mg, 1.70 mmol, 75% yield).

¹H NMR (DMSO-d6, δ [ppm]): 7.45-7.43 (m, 1H, Phenyl H2), 7.40-7.34 (m, 1H, Phenyl H4), 7.26-7.20 (m, 2H, Phenyl H5,6), 2.85-2.78 (m, 2H, Piperidine H2,6), 2.74-2.68 (m, 2H, Phenyl-CH₂—CH₂), 2.48-2.39 (partially covert by DMSO) (m, 2H, CH₂-CH₂—N), 1.95-1.86 (m, 2H, Piperidine H2,6), 1.85-1.75 (m, 1H, Piperidine H4), 1.73-1.65 (m, 2H, Piperidine H3,5), 1.52-1.39 (m, 2H, Piperidine H3,5).

¹³C NMR (DMSO-d6, δ [ppm]): 178.22 (—COO⁻), 144.20 (Phenyl 01), 131.82 (Phenyl C2), 130.70 (Phenyl C6), 129.02 (Phenyl C4), 128.24 (Phenyl C5), 121.88 (Phenyl C2), 60.33 (CH₂-CH₂—N), 53.87 (Piperidine C2,6), 44.05 (Piperidine C4), 32.76 (Phenyl-CH₂—CH₂), 29.87 (Piperidine C3,5).

MS (APCI, +, m/z): [M+H]⁺ 312.2+314.2

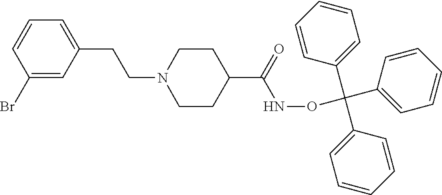 [SMILES: O=C(NOC(c1ccccc1)(c1ccccc1)c1ccccc1)C1CCN(CCc2cccc(Br)c2)CC1]

1-(3-Bromophenethyl)-N-(trityloxy)-piperidine-4-carboxamide (30): The title compound was prepared from 29 (457.9 mg, 1.44 mmol, 1.0 equiv) according to General Procedure F to provide 30 as a white solid (435.2 mg, 0.77 mmol, 53% yield).

¹H NMR (DMSO-d6, δ [ppm]): 10.30 (s, 1H, CO—NH—O), 7.42-7.40 (m, 1H, Phenyl C2), 7.38-7.26 (m, 16H, Phenyl H4+Trityl), 7.23-7.17 (m, 2H, Phenyl C5,6), 2.83-2.75 (m, 2H, Piperidine H2,6), 2.69-2.63 (m, 2H, Phenyl-CH₂—CH₂), 2.42-2.35 (m, 2H, CH₂-CH₂—N), 1.95-1.83 (m, 1H, Piperidine H4), 1.80-1.68 (m, 2H, Piperidine H2,6), 1.32-1.14 (m, 4H, Piperidine H3,5).

¹³C NMR (DMSO-d6, δ [ppm]): 172.92 (—CONH—), 144.00 (Phenyl C1), 142.86 (3× Phenyl C1 (Trityl)), 131.78 (Phenyl C2), 130.69 (Phenyl C6), 129.40 (Phenyl C2,6 or C3,5 (Trityl)), 129.06 (Phenyl C4), 128.22 (Phenyl C5), 127.89 (Phenyl C2,6 or C3,5 (Trityl)), 127.80 (3× Phenyl C4 (Trityl), 121.87 (Phenyl C3), 92.24 (O—C-Trityl), 59.91 (CH₂—CH₂—N), 52.91 (Piperidine C2,6), 39.33 (Piperidine C4 (HSQC)), 32.59 (Phenyl-CH₂—CH₂), 28.47 (Piperidine C3,5).

MS (APCI, +, m/z): [M+H]⁺ 569.2+571.2

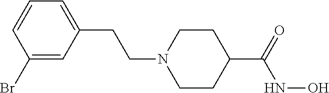 [SMILES: O=C(NO)C1CCN(CCc2cccc(Br)c2)CC1]

1-(3-Bromophenethyl)-N-hydroxypiperidine-4-carboxamide (DH71): The title compound was prepared from 30 (231.4 mg, 0.406 mmol, 1.0 equiv) according to General Procedure Ga to provide TFA salt of DH71 as a white solid (130.0 mg, 0.295 mmol, 73% yield as TFA salt).

¹H NMR (DMSO-d6, δ [ppm]): 10.63 (s, 1H, CO—NH—OH), 9.59 (s, 1H, CH₂—NH⁺-(Piperidine)), 7.55-7.52 (m, 1H, Phenyl H2), 7.48 (dt, J=7.2, 2.0 Hz, 1H, Phenyl H4), 7.35-7.27 (m, 2H, Phenyl H5,6), 3.65-3.52 (m, 2H, Piperidine H2,6), 3.41-3.22 (m, 2H, CH₂—CH₂—N), 3.01-2.87 (m, 4H, Phenyl-CH₂—CH₂+Piperidine H 2,6), 2.34-2.22 (m, 1H, Piperidine H4), 1.93-1.75 (m, 4H, Piperidine H3,5).

¹³C NMR (DMSO-d6, δ [ppm]): 170.12 (—CONH—), 159.16+158.82+158.48+158.15 (TFA), 140.21 (Phenyl C1), 131.91 (Phenyl C2), 131.23 (Phenyl C6), 130.20 (Phenyl C4), 128.36 (Phenyl C5), 122.28 (Phenyl C3), 56.69 (CH₂—CH₂—N), 51.51 (Piperidine C2,6), 36.83 (Piperidine C4), 29.38 (Phenyl-CH₂—CH₂), 26.19 (Piperidine C3,5).

HR-MS (ESI, m/z): [M+H]⁺ calcd for C₁₄H₂₀BrN₂O₂⁺: 327.0708+329.0801; found: 327.0700+329.0682.

Purity (HPLC, λ=210 nm): 97.3% (t_{R}=12.67 min)

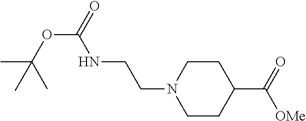 [SMILES: COC(=O)C1CCN(CCNC(=O)OC(C)(C)C)CC1]

Methyl 1-(2-((tert-butoxycarbonyl)amino)ethyl)-piperidine-4-carboxylate (31): The title compound was prepared from methyl piperidine-4-carboxylate (3000.0 mg, 20.97 mmol, 1.0 equiv) according to General Procedure Db to provide 31 as a yellow solid (3986.0 mg, 13.93 mmol, 66% yield).

¹H NMR (DMSO-d6, δ [ppm]): 6.64 (t, J=5.6 Hz, 1H, OCONH—CH₂—), 3.60 (s, 3H, —OCH₃), 3.01 (q, 6.4, 2H, —NH—CH₂—CH₂—), 2.81-2.73 (m, 2H, Piperidine H2,6), 2.33-2.23 (m, 3H, —CH₂—CH₂—N(Piperidine)+Piperidine H4), 2.02-1.91 (m, 2H, Piperidine H2,6), 1.81-1.73 (m, 2H, Piperidine H3,5), 1.59-1.46 (m, 2H, Piperidine H3,5), 1.37 (s, 9H, ((CH₃)₃—CH—).

¹³C NMR (DMSO-d6, δ [ppm]): 175.31 (—COOCH₃), 155.94 (—OCON—), 77.90 ((CH₃)₃—CH—O—), 57.94 (—CH₂—CH₂—N(Piperidine)), 52.78 (Piperidine C2,6), 51.80 (—O—CH₃), 40.59 (Piperidine C4), 37.89 (—NH—CH₂—CH₂—), 28.66 ((CH₃)₃—CH—), 28.42 (Piperidine C3,5).

MS (APCI, +, m/z): [M+H]⁺ 287.2

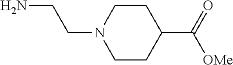 [SMILES: COC(=O)C1CCN(CCN)CC1]

Methyl 1-(2-aminoethyl)-piperidine-4-carboxylate (32): The title compound was prepared from 31 (2122.5 mg, 7.42 mmol, 1.0 equiv) according to General Procedure H to provide 32 as a yellow oil (use without further purification 7.42 mmol, 100% yield as 2×TFA salt).

¹H NMR (DMSO-d6, δ [ppm]): 10.12 (s, 1H, CH₂—NH-(Piperidine)), 8.20 (s, 3H, H₃N⁺ —CH₂), 3.65 (s, 3H, —OCH₃), 3.62-3.46 (m, 2H, Piperidine C2,6), 3.34-3.18 (m, 4H, H₃N⁺-CH₂—CH₂—), 3.15-2.95 (m, 2H, Piperidine H2,6), 2.76-2.58 (m, 1H, Piperidine H4), 2.18-1.96 (m, 2H, Piperidine H3,5), 1.87-1.67 (m, 2H, Piperidine H3,5).

¹³C NMR (DMSO-d6, δ [ppm]): 173.71 (—COOCH₃), 159.46+159.13+158.81+158.49 (TFA), 53.28 (H₃N⁺—CH₂—CH₂—), 52.27 (—OCH₃), 51.82 (Piperidine C2,6), 37.88 (Piperidine C4), 33.87 (H₃N⁺-CH₂—CH₂—), 25.76 (Piperidine C3,5).

MS (APCI, +, m/z): [M+H]⁺ 187.2

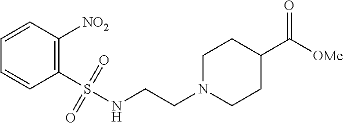 [SMILES: COC(=O)C1CCN(CCNS(=O)(=O)c2ccccc2[N+](=O)[O-])CC1]

Methyl 1-(2-((2-nitrophenyl)sulfonamido)ethyl)-piperidine-4-carboxylate (33): The title compound was prepared from 32 (3072.4 mg, 7.42 mmol, 1.0 equiv) according to following procedure to provide 33 as a yellow oil (1112 mg, 2.99 mmol, 82% yield). Primary amine (1 equiv.) and 2-nitrobenzenesulfonyl chloride (1.2 equiv.) were dissolved in THF, under cooling with ice water. After adding four equivalents of triethylamine, the reaction was stirred for 4 h at rt. Reaction was quenched by solvent evaporating and suspending the crude residue with water and dichloromethane. Aqueous layer was extracted with dichloromethane, for five times. Organic layer was dried over MgSO₄ before the solvent was evaporated under reduced pressure. Crude product was purified via flash column chromatography (CH₂Cl₂/MeOH).

¹H NMR (DMSO-d6, δ [ppm]): 8.09-8.03 (m, 1H, Nosyl H6), 8.02-7.96 (m, 1H, Nosyl H3), 7.90-7.77 (m, 3H, Nosyl H4,5+-SO₂NH—), 3.59 (s, 3H, —OCH₃), 3.03 (t, J=6.4 Hz, 2H, NH—CH₂—CH₂), 2.66-2.58 (m, 2H, Piperidine H2,6), 2.31 (t, J=6.4 Hz, 2H, CH₂—CH₂—N), 2.28-2.19 (m, 1H, Piperidine H4), 1.95-1.85 (m, 2H, Piperidine H2,6), 1.74-1.65 (m, 2H, Piperidine H3,5), 1.48-1-35 (m, 2H, Piperidine H3,5).

¹³C NMR (DMSO-d6, δ [ppm]): 175.21 (—COOCH₃), 148.00 (Nosyl C2) 134.36 (Nosyl C4), 133.44 (Nosyl C1), 133.07 (Nosyl C5), 129.99 (Nosyl C6), 124.87 (Nosyl C3), 57.15 (CH₂—CH₂—N), 52.50 (Piperidine C2,6), 51.79 (—OCH₃), 40.71 (CH₂—CH₂—N), 40.55 (Piperidine C4), 28.19 (Piperidine C3,5).

MS (APCI, +m/z): [M+H]⁺ 372.6

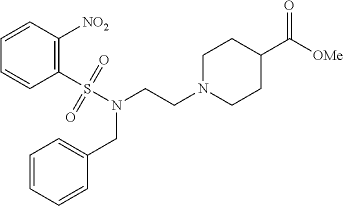 [SMILES: COC(=O)C1CCN(CCN(Cc2ccccc2)S(=O)(=O)c2ccccc2[N+](=O)[O-])CC1]

Methyl 1-(2-((N-benzyl-2-nitrophenyl)sulfonamido)ethyl)-piperidine-4-carboxylate (34): The title compound was prepared from 33 (1001.8 mg, 2.70 mmol, 1.0 equiv) according to General Procedure I to provide 34 as a yellow solid (852.1 mg, 1.85 mmol, 69% yield).

¹H NMR (DMSO-d6, δ [ppm]): 8.16 (dd, J=7.6, 1.6 Hz, 1H, Nosyl H6), 8.02 (dd, J=7.6, 1.6 Hz, 1H, Nosyl H3), 7.91 (td, J=7.6, 1.6 Hz, 1H, Nosyl H4), 7.85 (td, J=7.6, 1.6 Hz, 1H, Nosyl H5), 7.40-7.28 (m, 5H, Phenyl), 4.57 (s, 2H, Phenyl-CH₂—N), 3.58 (s, 3H, —OCH₃), 3.29 (t, J=6.6 Hz, 2H, N—CH₂—CH₂—N), 2.62-2.53 (m, 2H, Piperidine H2,6), 2.26-2.16 (m, 3H, N—CH₂—CH₂—N+Piperidine H4), 1.88-1.77 (m, 2H, Piperidine H2,6), 1.72-1.62 (m, 2H, Piperidine H3,5), 1.45-1.33 (m, 2H, Piperidine H3,5).

¹³C NMR (DMSO-d6, δ [ppm]): 175.17 (—COO⁻), 147.95 (Nosyl C2), 136.80 (Phenyl C1), 134.83 (Nosyl C4), 132.88 (Nosyl C5), 132.79 (Nosyl C1), 130.13 (Nosyl C6), 128.97 (Phenyl C3,5), 128.31 (Phenyl C2,6), 128.15 (Phenyl C4), 124.75 (Nosyl C3), 56.13 (N—CH₂—CH₂—N), 52.61 (Piperidine C2,6), 51.88 (Benzyl-CH₂—N), 51.80 (O-CH₃), 44.86 (N-CH₂—CH₂—N), 40.51 (Piperidine C4), 28.22 (Piperidine C3,5).

MS (APCI, +, m/z): [M+H]⁺ 462.8

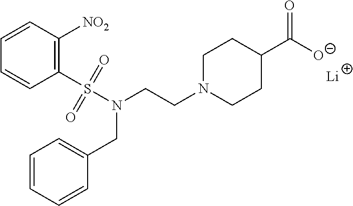 [SMILES: O=C([O-])C1CCN(CCN(Cc2ccccc2)S(=O)(=O)c2ccccc2[N+](=O)[O-])CC1 ; [Li+]]

Lithium 1-(2-((N-Benzyl-2-nitrophenyl)sulfonamido)ethyl)-piperidine-4-carboxylate (35): The title compound was prepared from 34 (852.1 mg, 1.85 mmol, 1.0 equiv) according to General Procedure E to provide 35 as a yellow solid (use without further purification 1.85 mmol, 100% yield as Li⁺ salt).

¹H NMR (DMSO-d6, δ [ppm]): Signal nicht sichtbar, da Li-Salz (—COOH), 8.22 (dd, J=7.6, 1.6 Hz, 1H, Nosyl H6), 8.02 (dd, J=7.6, 1.6 Hz, 1H, Nosyl H3), 7.91 (td, J=7.6, 1.6 Hz, 1H, Nosyl H4), 7.85 (td, J=7.6, 1.6 Hz, 1H, Nosyl H4), 7.39-7.24 (m, 5H, 5× Phenyl H), 4.56 (s, 2H, Phenyl-CH₂—N), 3.29 (t, J=6.8 Hz, 2H, N—CH₂—CH₂—N), 2.17 (t, J=6.8 Hz, 2H, N—CH₂—CH₂—N), 1.78-1.66 (m, 2H, Piperidine C2,6), 1.65-1.55 (m, 3H, Piperidine H3,5+H4), 1.45-1.31 (m, 2H, Piperidine H3,5).

¹³C NMR (DMSO-d6, δ [ppm]): 174.54 (—COO⁻), 147.99 (Nosyl C2), 136.77 (Phenyl C1), 134.86 (Nosyl C4), 132.92 (Nosyl C5), 132.78 (Nosyl C1), 130.18 (Nosyl C6), 128.98 (Phenyl C3,5), 128.30 (Phenyl C2,6), 128.15 (Phenyl C4), 124.75 (Nosyl C3), 56.67 (N—CH₂—CH₂—N), 53.97 (Piperidine C2,6), 51.85 (Phenyl-CH₂—), 44.72 (N-CH₂—CH₂—N), 44.09 (Piperidine C4), 29.87 (Piperidine C3,5).

MS (APCI, +, m/z): [M+H]⁺ 448.2

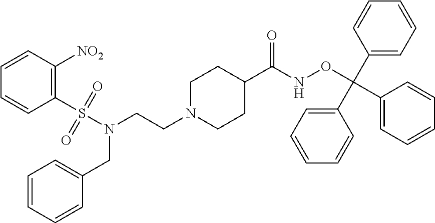 [SMILES: O=C(NOC(c1ccccc1)(c1ccccc1)c1ccccc1)C1CCN(CCN(Cc2ccccc2)S(=O)(=O)c2ccccc2[N+](=O)[O-])CC1]

1-(2-((N-Benzyl-2-nitrophenyl)sulfonamido)ethyl)-N-(trityloxy)-piperidine-4-carboxamide (36): The title compound was prepared from 35 (838.7 mg, 1.85 mmol, 1.0 equiv) according to General Procedure F to provide 36 as a yellow solid (535.8 mg, 0.760 mmol, 41% yield).

¹H NMR (DMSO-d6, δ [ppm]): 10.27 (s, 1H, CONH—O), 8.17 (dd, J=7.6, 1.2 Hz, 1H Nosyl H6), 8.00 (dd, J=7.6, 1.2 Hz, 1H, Nosyl H3), 7.89 (td, J=7.6, 1.2 Hz, 1H, Nosyl H4), 7.81 (td, J=7.6, 1.2 Hz, 1H, Nosyl H5), 7.38-7.21 (m, 20H, 5× Phenyl+15× Trityl), 4.52 (s, 2H, N—CH₂-Phenyl), 3.24 (t, J=6.4 Hz, 2H, N—CH₂—CH₂—N), 2.48 (2H, Piperidine C2,6, HSQC), 2.12 (t, J=6.4 Hz, 2H, N—CH₂—CH₂—N), 1.87-1.76 (m, 1H, Piperidine H4), 1.65-1.53 (m, 2H, Piperidine H2,6), 1.20-1.03 (m, 4H, Piperidine H3,5).

¹³C NMR (DMSO-d6, δ [ppm]): 172.79 (CONH), 147.96 (Nosyl C2), 142.84 (Trityl C1), 136.72 (Phenyl C1), 134.86 (Nosyl C4), 132.84 (Nosyl C5), 132.67 (Nosyl C1), 130.18 (Nosyl C6), 129.38 (Trityl C2,6 or C3,6), 128.94 (Phenyl C3,5), 128.30 (Phenyl C2,6), 128.14 (Phenyl C4), 127.90 (Trityl C2,6 or C3,5), 127.80 (Trityl C4), 124.72 (Nosyl C3), 92.22 (O—C-Trityl), 56.23 (N—CH₂—CH₂—N), 52.88 (Piperidine C2,6), 51.73 (N-CH₂-Phenyl), 44.73 (N-CH₂—CH₂—N), 39.13 (Piperidine C4), 28.30 (Piperidine C3,5).

HR-MS (ESI, m/z): [M+H]⁺ 705.2739

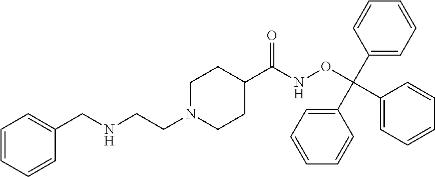 [SMILES: O=C(NOC(c1ccccc1)(c1ccccc1)c1ccccc1)C1CCN(CCNCc2ccccc2)CC1]

1-(2-(Benzylamino)ethyl)-N-(trityloxy)-piperidine-4-carboxamide (37): The title compound was prepared from 36 (331.8 mg, 0.44 mmol, 1.0 equiv) according to General Procedure J to provide 37 as a colorless crystalline solid (158.5 mg, 0.305 mmol, 69% yield).

¹H NMR (DMSO-d6, δ [ppm]): 10.29 (s, 1H, CONH—), 7.41-7.18 (m, 20H, 5× Phenyl+15× Trityl), 3.67 (s, 2H, Phenyl-CH₂—N), 2.72-2.63 (m, 2H, Piperidine H2,6), 2.49 (2H, N—CH₂—CH₂—N, HSQC), 2.28 (t, J=6.4 Hz, 2H, N—CH₂—CH₂—N), 1.89 (q, J=8.0 Hz, 1H, Piperidine H4), 1.75-1.61 (m, 2H, Piperidine H2,6), 1.31-1.15 (m, 4H, Piperidine H3,5).

¹³C NMR (DMSO-d6, δ [ppm]): 172.95 (CONH), 142.85 (Trityl C1), 141.10 (Phenyl C1), 129.39 (Trityl C2,6 or 3,5), 128.51 (Phenyl C3,5), 128.33 (Phenyl C2,6), 127.89 (Trityl C2,6 or C3,5), 127.79 (Trityl C4), 126.98 (Phenyl C4), 92.22 (O—C-Trityl), 57.98 (N—CH₂—CH₂—N), 53.29 (Phenyl-C—N), 53.15 (Piperidine C2,6), 45.88 (N-CH₂—CH₂—N), 39.64 (Piperidine C4), 28.50 (Piperidine C3,5).

HR-MS (ESI, m/z): [M+H]⁺ 520.2957

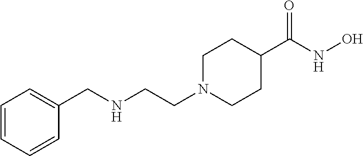 [SMILES: O=C(NO)C1CCN(CCNCc2ccccc2)CC1]

1-(2-(Benzylamino)ethyl)-N-hydroxypiperidine-4-carboxamide (DH79): The title compound was prepared from 37 (103.0 mg, 0.198 mmol, 1.0 equiv) according to General Procedure Gb to provide 2×TFA salt of DH79 as a colorless oil (100.0 mg, 0.198 mmol, 100% yield as 2×TFA salt).

¹H NMR (DMSO-d6, δ [ppm]): 10.64 (s, 1H, CO—NH—OH), 9.62 (bs, 1H, CH₂—NH⁺ (Piperidine)), 9.28 (bs, 2H, CH₂—NH₂⁺—CH₂ or CO—NH—OH), 7.58-7.41 (m, 5H, Phenyl), 4.22 (s, 2H, Phenyl-CH₂—NH₂⁺), 3.65-3.47 (m, 2H, Piperidine H2,6), 3.46-3.36 (m, 4H, NH₂⁺—CH₂—CH₂-(Piperidine)), 3.12-2.94 (m, 2H, Piperidine H2,6), 2.36-3.23 (m, 1H, Piperidine H4), 2.00-1.71 (m, 4H, Piperidine H3,5).

¹³C NMR (DMSO-d6, δ [ppm]): 170.05 (—CONH—), 159.41+159.07+158.73+158.39 (TFA), 132.07 (Phenyl C1), 130.26 (Phenyl C2,6), 129.63 (Phenyl C4), 129.25 (Phenyl), 52.08 (Piperidin C2,6, NH₂⁺—CH₂—CH₂-(Piperidine), 50.78 (Phenyl-CH₂—NH₂⁺), 41.17 (NH₂⁺—CH₂—CH₂-(Piperidine)), 36.47 (Piperidine C4), 26.25 (Piperidine C3,5).

HR-MS (ESI, m/z): [M+H]⁺ calcd for C₁₅H₂₄N₃O₂⁺: 278.1869; found: 278.1864.

Purity (HPLC, λ=210 nm): 95.7% (t_{R}=8.657 min) (modification of method: flow rate 0.5 mL/min)

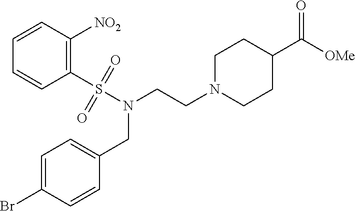 [SMILES: COC(=O)C1CCN(CCN(Cc2ccc(Br)cc2)S(=O)(=O)c2ccccc2[N+](=O)[O-])CC1]

Methyl 1-(2-((N-(4-bromobenzyl)-2-nitrophenyl)sulfonamido)ethyl)-piperidine-4-carboxylate (38): The title compound was prepared from 33 (500.0 mg, 1.35 mmol, 1.0 equiv) according to General Procedure I to provide 38 as a green oil (480.4 mg, 0.889 mmol, 66% yield).

¹H NMR (DMSO-d6, δ [ppm]): 8.16 (dd, J=8.0, 1.2 Hz, 1H, Nosyl H6), 8.02 (dd, J=8.0, 1.2 Hz, 1H, Nosyl H3), 7.91 (td, J=8.0, 1.2 Hz, 1H, Nosyl H4), 7.85 (td, J=8.0, 1.2 Hz, 1H, Nosyl H5), 7.60-7.54 (m, 2H, Phenyl H3,5), 7.32-7.26 (m, 2H, Phenyl H2,6), 4.54 (s, 2H, Phenyl-CH₂—N), 3.59 (s, 3H, —OCH₃), 3.31 (t, J=6.4 Hz, 2H, N—CH₂—CH₂—N), 2.63-2.53 (m, 2H, Piperidine C2,6), 2.27-2.16 (m, 3H, N—CH₂-CH₂—N+Piperidine H4), 1.90-1.78 (m, 2H, Piperidine H2,6), 1.73-1.63 (m, 2H, Piperidine H3,5), 1.45-1.36 (m, 2H, Piperidine H3,5).

¹³C NMR (DMSO-d6, δ [ppm]): 175.16 (CON), 147.96 (Nosyl C2), 136.55 (Phenyl 01), 134.90 (Nosyl C4), 132.92 (Nosyl C5), 132.61 (Nosyl 01), 131.83 (Phenyl C3,5), 130.43 (Phenyl C2,6), 130.13 (Nosyl C6), 124.79 (Nosyl C3), 121.22 (Phenyl C4), 56.14 (N—CH₂-CH₂—N), 52.59 (Piperidine C2,6), 51.80 (—OCH₃), 51.32 (Phenyl-CH₂—N), 45.16 (N-CH₂—CH₂—N), 40.47 (Piperidine C4, HSQC), 28.20 (Piperidine C3,5).

MS (APCI, +): [M+H]⁺ 540.1+542.1

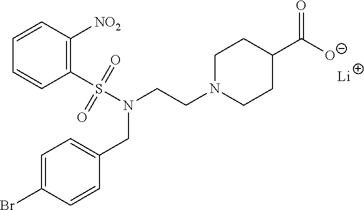 [SMILES: O=C([O-])C1CCN(CCN(Cc2ccc(Br)cc2)S(=O)(=O)c2ccccc2[N+](=O)[O-])CC1 ; [Li+]]

Lithium 1-(2-((N-(4-bromobenzyl)-2-nitrophenyl)sulfonamido)ethyl)-piperidine-4-carboxylate (39): The title compound was prepared from 38 (468.0 mg, 0.865 mmol, 1.0 equiv) according to General Procedure E to provide 39 as a yellow solid (134.8 mg, 0.256 mmol, 30% yield as Li⁺ salt).

¹H NMR (DMSO-d6, δ [ppm]): 8.21 (dd, J=8.0, 1.6 Hz, 1H, Nosyl H6), 8.01 (dd, J=8.0, 1.6 Hz, 1H, Nosyl H3), 7.91 (td, J=8.0, 1.6 Hz, 1H, Nosyl H4), 7.85 (td, J=8.0, 1.6 Hz, 1H, Nosyl H5), 7.58-7.53 (m, 2H, Phenyl H3,5), 7.31-7.25 (m, 2H, Phenyl H2,6), 4.54 (s, 2H, Phenyl-CH₂—N), 3.30 (t, J=6.4 Hz, 2H, N—CH₂—CH₂—N), 2.60-5.53 (m, 2H, Piperidine H2,6), 2.19 (t, J=6.4 Hz, 2H, N—CH₂—CH₂—N), 1.79-1.67 (m, 3H, Piperidine H2,6+H4), 1.63-1.58 (m, 2H, Piperidine H3,5), 1.45-1.32 (m, 2H, Piperidine H3,5).

¹³C NMR (DMSO-d6, δ [ppm]): 179.31 (COO⁻), 147.99 (Nosyl C2), 136.51 (Phenyl C1), 134.95 (Nosyl C4), 132.96 (Nosyl C5), 132.60 (Nosyl C1), 131.84 (Phenyl C3,5), 130.43 (Phenyl C2,6), 130.19 (Nosyl C6), 124.80 (Nosyl C3), 121.22 (Phenyl C4), 56.63 (N—CH₂—CH₂—N), 53.93 (Piperidine C2,6), 51.23 (Phenyl-CH₂—N), 45.00 (N-CH₂—CH₂—N), 44.02 (Piperidine C4), 29.81 (Piperidine C3,5).

MS (APCI, +): [M+H]⁺ 526.0+528.0

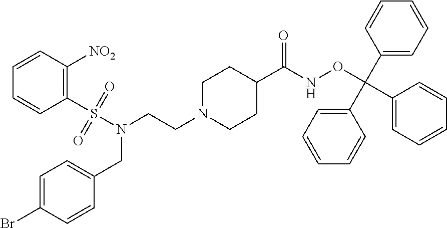 [SMILES: O=C(NOC(c1ccccc1)(c1ccccc1)c1ccccc1)C1CCN(CCN(Cc2ccc(Br)cc2)S(=O)(=O)c2ccccc2[N+](=O)[O-])CC1]

1-(2-((N-(4-Bromobenzyl)-2-nitrophenyl)sulfonamido)ethyl)-N-(trityloxy)-piperidine-4-carboxamide (40): The title compound was prepared from 39 (134.8 mg, 0.256 mmol, 1.0 equiv) according to General Procedure F to provide 40 as a yellow oil (136.4 mg, 0.174 mmol, 68% yield).

¹H NMR (DMSO-d6, δ [ppm]): 10.27 (s, 1H, CONH), 8.19-8.13 (m, 1H, Nosyl H6), 8.00 (dd, J=8.0, 1.2 Hz, 1H, Nosyl H3), 7.94-7.86 (m, 1H, Nosyl H4), 7.86-7.78 (m, 1H, Nosyl H5), 7.57-7.52 (m, 2H, Phenyl H3,5), 7.41-7.22 (m, 17H, Phenyl H2,6+15× Trityl), 4.50 (s, 2H, Phenyl-CH₂—N), 3.26 (t, J=6.4 Hz, 2H, N—CH₂—CH₂—N), 2.53 (m, 2H, Piperidine H2,6, HSQC), 2.14 (t, J=6.4 Hz, 2H, N—CH₂—CH₂—N), 1.89-1.76 (m, 1H, Piperidine H4), 1.65-1.53 (m, 2H, Piperidine H2,6), 1.21-1.04 (m, 4H, Piperidine H3,5).

¹³C NMR (DMSO-d6, δ [ppm]): 172.79 (CONH), 147.97 (Nosyl C2), 142.84 (Trityl C1), 136.47 (Phenyl C1), 134.93 (Nosyl C4), 132.88 (Nosyl C5), 132.49 (Nosyl C1), 131.80 (Phenyl C3,5), 130.43 (Phenyl C2,6), 130.20 (Nosyl C6), 129.39 (Trityl C2,6 or C3,5), 127.90 (Trityl C2,6 or C3,5), 127.80 (Trityl C4), 124.77 (Nosyl C3), 121.21 (Phenyl C4), 56.21 (N—CH₂-CH₂—N), 52.86 (Piperidine C2,6), 51.14 (Phenyl-CH₂—N), 45.04 (N-CH₂—CH₂—N), 38.92 (Piperidine C4), 28.30 (Piperidine C3,5).

HR-MS (ESI, m/z): [M+H]⁺ 783.1838+785.1820

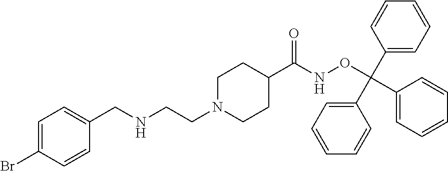 [SMILES: O=C(NOC(c1ccccc1)(c1ccccc1)c1ccccc1)C1CCN(CCNCc2ccc(Br)cc2)CC1]

1-(2-((4-Bromobenzyl)amino)ethyl)-N-(trityloxy)-piperidine-4-carboxamide (41): The title compound was prepared from 40 (127.7 mg, 0.163 mmol, 1.0 equiv) according to General Procedure J to provide 41 as a colorless oil (71.0 mg, 0.119 mmol, 73% yield).

¹H NMR (DMSO-d6, δ [ppm]): 10.29 (s, 1H, CONH), 7.51-7.46 (m, 2H, Phenyl H3,5), 7.42-7.19 (m, 17, Phenyl 2,6+15× Trityl), 3.64 (s, 2H, Phenyl-CH₂—N), 2.71-2.62 (m, 2H, Piperidine H2,6), 2.49-2.45 (m, 2H, N—CH₂—CH₂—N), 2.27 (t, J=6.4 Hz, 2H, N—CH₂-CH₂—N), 1.95-1.82 (m, 1H, Piperidine H4), 1.73-1.60 (m, 2H, Piperidine H2,6), 1.32-1.14 (s, 4H, Piperidine H3,5).

¹³C NMR (DMSO-d6, δ [ppm]): 172.95 (CON), 142.85 (Trityl C1), 140.87 (Phenyl C1), 131.33 (Phenyl C3,5), 130.49 (Phenyl C2,6), 129.39 (Trityl C2,6 or C3,5), 127.89 (Trityl C2,6 or C3,5), 127.79 (Trityl C4), 92.22 (O—C-Trityl), 58.10 (N—CH₂-CH₂—N), 53.18 (Piperidine C2,6), 52.52 (Phenyl-C—N), 45.87 (N-CH₂—CH₂—N), 40.01 (Piperidine C4, HSQC), 28.50 (Piperidine C3,5).

MS (APCI, +): [M+H]⁺ 598.2+600.2

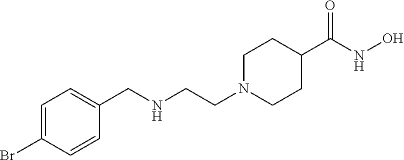 [SMILES: O=C(NO)C1CCN(CCNCc2ccc(Br)cc2)CC1]

1-(2-((4-Bromobenzyl)amino)ethyl)-N-hydroxypiperidine-4-carboxamide (DH88): The title compound was prepared from 41 (65.8 mg, 0.11 mmol, 1.0 equiv) according to General Procedure Ga to provide the 2×TFA salt of DH88 as a brown oil (30.3 mg, 0.064 mmol, 47% yield as 2×TFA salt).

¹H NMR (DMSO-d6, δ [ppm]): 10.65 (s, 1H, CO—NH—OH), 9.73 (s, 1H, CH₂—NH⁺-(Piperidine)), 9.37 (s, 2H, CH₂—NH₂⁺—CH₂ or CO—NH—OH), 7.69 (d, J=8.4 Hz, 2H, Phenyl H3,5), 7.46 (d, J=8.4 Hz, 2H, Phenyl H2,6), 4.21 (s, 2H, Phenyl-CH₂—NH₂), 3.63-3.48 (m, 2H, Piperidine H2,6), 3.39 (s, 4H, NH₂⁺—CH₂—CH₂-(Piperidine)), 3.12-2.93 (m, 2H, Piperidine H2,6), 2.36-2.22 (m, 1H, Piperidine H4), 1.98-1.70 (d, 4H, Piperidine H3,5).

¹³C NMR (DMSO-d6, δ [ppm]): 170.05 (—CONH—), 159.40+159.06+158.72+158.40 (TFA), 132.57 (Phenyl C2,6), 132.16 (Phenyl C3,5), 131.41 (Phenyl C1), 123.06 (Phenyl C4), 52.05 (Piperidine C2,6, NH₂⁺—CH₂—CH₂-(Piperidine)), 49.94 (Phenyl-CH₂—NH₂⁺), 41.08 (NH₂⁺—CH₂—CH₂-(Piperidine)), 36.48 (Piperidine C4), 26.23 (Piperidine C3,5).

HR-MS (ESI, m/z): [M+H]⁺ calcd for C₁₅H₂₃BrN₃O₂⁺: 356.0974+358.1070; found: 356.0963+358.0948.

Purity (HPLC, λ=210 nm): 95.4% (t_{R}=9.95 min)

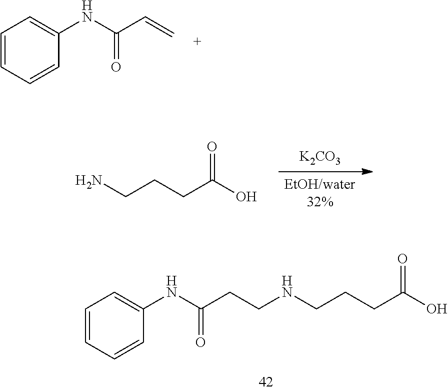 [SMILES: C=CC(=O)Nc1ccccc1 ; NCCCC(=O)O ; "K2CO3" ; "EtOH/water32%" ; O=C(O)CCCNCCC(=O)Nc1ccccc1]

4-((3-Oxo-3-(phenylamino)propyl)amino)butanoic acid (42): To a stirred solution of gamma-aminobutyric acid (3.14 g, 29.90 mmol, 2.2 equiv) and K₂CO₃ (2.68 g, 19.40 mmol, 1.4 equiv) in water (27 mL)/EtOH (17 mL) heated to 70° C., was added dropwise a solution of N-phenylacrylamide (2.00 g, 13.59 mmol, 1.0 equiv) in EtOH (10 mL). The reaction was stirred at 70° C. for 3.5 h, concentrated and extracted with CH₂Cl₂ (5×20 mL). Organic layers were discarded and the aqueous phase was acidified with HCl, then evaporated to dryness to yield a white solid, which was recrystallized by dissolving in a minimal amount of water/MeOH at 50° C. and then concentrating under reduced pressure at 50° C. until precipitation was visible, followed by slowly cooling to 0° C. overnight. Crystals were filtered and washed once with ice cold water, then with acetone, to provide the HCl salt of 42 as white crystals (1.239 g, 4.32 mmol, 32% yield).

TLC R_{f} 0.05 (10% water in MeCN).

¹H NMR (400 MHz, MeOD-d₄) δ 7.61-7.55 (m, 2H), 7.34-7.27 (m, 2H), 7.15-7.05 (m, 1H), 3.36 (t, J=6.4 Hz, 2H), 3.18-3.09 (m, 2H), 2.87 (t, J=6.4 Hz, 2H), 2.49 (t, J=7.1 Hz, 2H), 2.00 (app p, J=7.1 Hz, 2H) ppm.

LC/MS (m/z): [M+H]⁺ 251.2, [M−H]⁻ 249.2

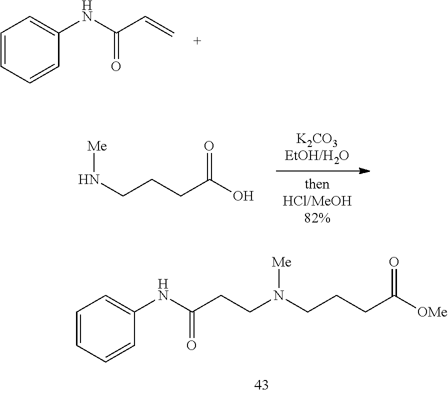 [SMILES: C=CC(=O)Nc1ccccc1 ; CNCCCC(=O)O ; "K2CO3EtOH/H2O" ; "thenHCl/MeOH82%" ; COC(=O)CCCN(C)CCC(=O)Nc1ccccc1]

Methyl 4-(methyl(3-oxo-3-(phenylamino)propyl)amino)butanoate (43): N-phenylacrylamide (2.00 g, 13.59 mmol, 1.0 equiv), N-Me-gamma-aminobutyric acid.HCl (2.30 g, 14.95 mmol, 1.1 equiv) and K₂CO₃ (3.76 g, 27.18 mmol, 2.0 equiv) were suspended in water/EtOH (1:1, 40 mL). The reaction mixture was stirred at 70° C. for 7 h, then cooled to RT, acidified to pH ˜1 with 3 M HCl and evaporated to dryness. The residue was suspended in MeOH (25 mL) and stirred at rt for 3 days, then concentrated in vacuo and purified by MPLC (80 g silica, gradient: 0→10% MeOH in CH₂Cl₂) to provide the HCl salt of 43 as a yellow amorphous solid, which crystallized over time into a yellow/green solid (3.49 g, 11.09 mmol, 82% yield).

TLC R_{f} 0.4 (10% MeOH in CH₂Cl₂).

¹H NMR (400 MHz, MeOD-d₄) δ 7.64-7.54 (m, 2H), 7.34-7.28 (m, 2H), 7.13-7.07 (m, 1H), 3.69 (s, 3H), 3.60 (br s, 1H), 3.45 (br s, 1H), 3.26 (br s, 2H), 2.96 (t, J=6.6 Hz, 2H), 2.93 (s, 3H), 2.52 (t, J=7.0 Hz, 2H), 2.13-2.02 (m, 2H) ppm.

¹³C NMR (101 MHz, MeOD-d₄) δ 172.3, 167.9, 137.4, 127.7, 123.3, 119.0, 54.8, 51.2, 50.2, 38.9, 29.1 (2CH₂), 18.3 ppm.

LC/MS (m/z): [M+H]⁺ 279.2.

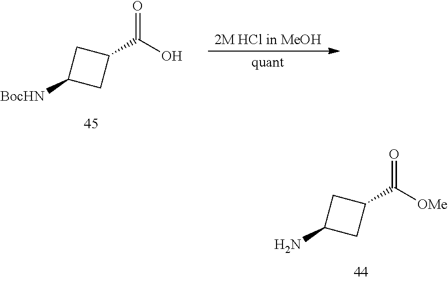 [SMILES: CC(C)(C)OC(=O)N[C@H]1C[C@H](C(=O)O)C1 ; "2M HCl in MeOH" ; "quant" ; COC(=O)[C@H]1C[C@H](N)C1]

trans-Methyl 3-aminocyclobutanecarboxylate (44): 45 (0.600 g, 2.79 mmol, 1.0 equiv) was dissolved in 2 M HCl in MeOH (20 mL, 40 mmol, 14.4 equiv) and stirred at rt over night, then concentrated to dryness and coevaporated with MeOH to provide the HCl salt of 44 as slightly yellow solid (0.483 g, 2.92 mmol, quant yield).

TLC R_{f} 0.18 (20% MeOH in CH₂Cl₂).

¹H NMR (400 MHz, MeOD-d₄) δ 3.99-3.87 (m, 1H), 3.72 (s, 3H), 3.30-3.22 (m, 1H), 2.67-2.58 (m, 2H), 2.52-2.42 (m, 2H) ppm.

LC/MS (m/z): [M+H]⁺ 130.2.

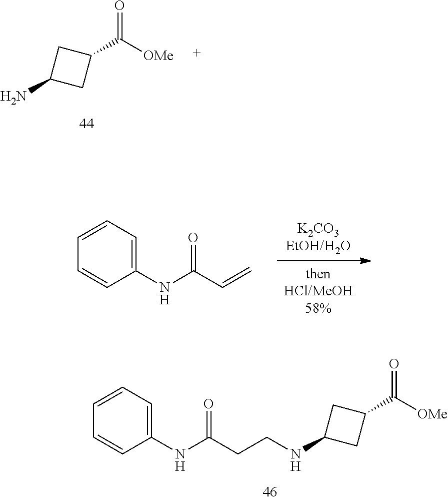 [SMILES: COC(=O)[C@H]1C[C@H](N)C1 ; C=CC(=O)Nc1ccccc1 ; "K2CO3EtOH/H2O" ; "thenHCl/MeOH58%" ; COC(=O)[C@H]1C[C@H](NCCC(=O)Nc2ccccc2)C1]

trans-Methyl 3-((3-oxo-3-(phenylamino)propyl)amino)cyclobutanecarboxylate (46): 44.HCl (0.165 g, 1.00 mmol, 1.0 equiv), N-phenylacrylamide (0.155 g, 1.05 mmol, 1.05 equiv) and K₂CO₃ (0.276 g, 2.00 mmol, 2.0 equiv) were dissolved in water/EtOH (1:1, 10 mL). The reaction mixture was stirred at 80° C. for 38 h, then cooled to rt, diluted with 1 M HCl (40 mL), extracted with EtOAc (2×30 mL) and the aqueous layer evaporated to dryness. The residue was suspended in MeOH (25 mL), acidified with HCl in MeOH, stirred at rt for 3 h, then concentrated to dryness. The residue was dissolved in dilute K₂CO₃ solution (40 mL) and extracted with CH₂Cl₂ (3×25 mL), dried (MgSO₄) and concentrated in vacuo. The crude product was purified by MPLC (24 g silica, gradient: CH₂Cl₂ for 1 CV, then 0→10% MeOH in CH₂Cl₂ over 10 CV) to provide 46 as a slightly yellow oil (0.160 g, 0.578 mmol, 58% yield).

TLC R_{f} 0.68 (20% MeOH in CH₂Cl₂).

¹H NMR (400 MHz, CDCl₃) δ 10.00 (s, 1H), 7.54-7.46 (m, 2H), 7.34-7.27 (m, 2H), 7.07 (tt, J=7.4, 1.2 Hz, 1H), 3.70 (s, 3H), 3.65-3.53 (m, 1H), 3.15-3.03 (m, 1H), 2.96-2.82 (m, 2H), 2.61-2.51 (m, 2H), 2.51-2.45 (m, 2H), 2.14-2.03 (m, 2H), 1.66 (s, 1H) ppm.

LC/MS (m/z): [M+H]⁺ 277.2.

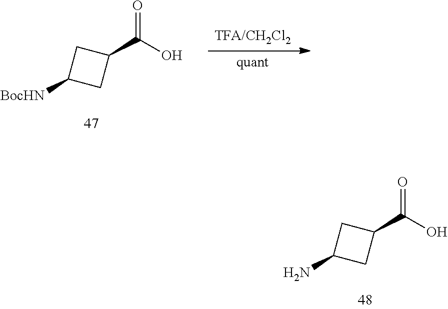 [SMILES: CC(C)(C)OC(=O)N[C@H]1C[C@@H](C(=O)O)C1 ; "TFA/CH2Cl2" ; "quant" ; N[C@H]1C[C@@H](C(=O)O)C1]

cis-3-Aminocyclobutanecarboxylic acid (48): To a stirred suspension of 47 (0.402 g, 1.87 mmol, 1.0 equiv) in CH₂Cl₂ (12 mL), cooled to 0° C., was added TFA (3.0 mL, 39.17 mmol, 21 equiv), then allowed to warm to rt within 3.5 h. The reaction mixture was concentrated to dryness and coevaporated with CH₂Cl₂ and MeOH provide the TFA salt of 48 as colorless crystals (0.434 g, 1.89 mmol, quant yield).

TLC R_{f} 0.18 (20% MeOH in CH₂Cl₂).

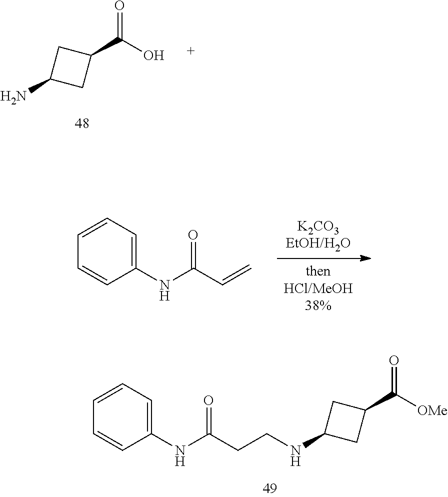 [SMILES: N[C@H]1C[C@@H](C(=O)O)C1 ; C=CC(=O)Nc1ccccc1 ; "K2CO3EtOH/H2O" ; "thenHCl/MeOH38%" ; COC(=O)[C@H]1C[C@@H](NCCC(=O)Nc2ccccc2)C1]

cis-Methyl 3-((3-oxo-3-(phenylamino)propyl)amino)cyclobutanecarboxylate (49): 48.TFA (0.243 g, 1.06 mmol, 1.0 equiv), N-phenylacrylamide (0.172 g, 1.17 mmol, 1.1 equiv) and K₂CO₃ (0.330 g, 2.39 mmol, 2.25 equiv) were dissolved in water/EtOH (1:1, 8 mL) and stirred at 75° C. for 24 h, then cooled to rt, diluted with 1 M HCl (30 mL), extracted with EtOAc (2×25 mL) and the aqueous layer evaporated to dryness. The residue was suspended in MeOH (25 mL), acidified with HCl (2 M in MeOH, 1 mL), stirred at rt for 4 h, then concentrated to dryness. The residue was dissolved in dilute K₂CO₃ solution (30 mL) and extracted with CH₂Cl₂ (4×20 mL), dried (MgSO₄) and concentrated in vacuo. The crude product was purified by MPLC (24 g silica, gradient: 0→5% MeOH in CH₂Cl₂ over 9 CV, then 5% MeOH over 8 CV) to provide 49 as a colorless oil (0.111 g, 0.402 mmol, 38% yield).

TLC R_{f} 0.40 (20% MeOH in CH₂Cl₂).

¹H NMR (600 MHz, CDCl₃) δ 10.13 (s, 1H), 7.54-7.49 (m, 2H), 7.32-7.27 (m, 2H), 7.06 (tt, J=7.4, 1.2 Hz, 1H), 3.69-3.67 (m, 3H), 3.32-3.25 (m, 1H), 2.91-2.86 (m, 2H), 2.86-2.78 (m, 1H), 2.59-2.52 (m, 2H), 2.49-2.43 (m, 2H), 2.09-2.01 (m, 2H), 1.76-1.70 (m, 1H) ppm.

LC/MS (m/z): [M+H]⁺ 277.2.

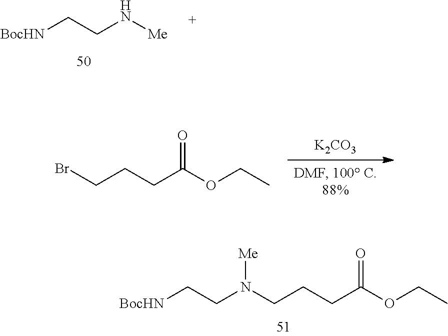

Ethyl 4-((2-((tert-butoxycarbonyl)amino)ethyl)(methyl)amino)butanoate (51): To a stirred suspension of 50 (1.52 g, 8.74 mmol, 1.0 equiv) and K₂CO₃ (2.42 g, 17.49 mmol, 2.0 equiv) in DMF (9 mL) was added ethyl 4-bromobutyrate (1.38 mL, 9.62 mmol, 1.1 equiv). The reaction mixture was stirred at 100° C. for 16 h, then cooled to rt, poured into water (100 mL) and extracted with EtOAc (3×100 mL). Combined organic layers were dried (MgSO₄), filtered and concentrated in vacuo. The crude product was purified by FCC (280 g silica, eluent: 8%, then 20% MeOH in CH₂Cl₂) to provide 51 as brown oil (2.21 g, 7.67 mmol, 88% yield).

TLC R_{f} 0.41 (10% MeOH in CH₂Cl₂).

¹H NMR (400 MHz, MeOD-d₄) δ 4.12 (q, J=7.1 Hz, 2H), 3.16 (t, J=6.8 Hz, 2H), 2.48 (t, J=6.8 Hz, 2H), 2.46-2.41 (m, 2H), 2.35 (t, J=7.2 Hz, 2H), 2.26 (s, 3H), 1.78 (app p, J=7.2 Hz, 2H), 1.44 (s, 9H), 1.25 (t, J=7.1 Hz, 3H) ppm.

LC/MS (m/z): [M+H]⁺ 289.2.

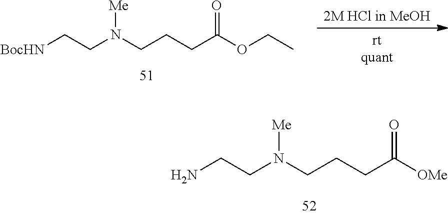 [SMILES: CCOC(=O)CCCN(C)CCNC(=O)OC(C)(C)C ; "2M HCl in MeOH" ; "rtquant" ; COC(=O)CCCN(C)CCN]

Methyl 4-((2-aminoethyl)(methyl)amino)butanoate (52): 51 (1.80 g, 6.27 mmol, 1 equiv) was dissolved in 2 M HCl in MeOH (8 mL, 156.74 mmol, 25 equiv) and stirred at rt for 26 h. The solvent was removed in vacuo and residual acid was removed by coevaporation with MeOH (3×10 mL) and high vacuum to provide the 2.HCl salt of 52 as a yellow amorphous solid (1.54 g, 6.24 mmol, quant yield). The product was stored as a stock solution in MeOH and used without further purification.

TLC R_{f} 0.08 (20% MeOH in CH₂Cl₂), 0.21 (20% MeOH and 0.5% NH₄OH in CH₂Cl₂)

¹H NMR (400 MHz, MeOD-d₄) δ 3.70 (s, 3H), 3.62-3.43 (m, 4H, 2CH₂), 3.39-3.23 (m, 2H, CH₂), 2.98 (s, 3H), 2.53 (t, J=7.0 Hz, 2H), 2.17-2.04 (m, 2H) ppm.

¹³C NMR (101 MHz, MeOD-d₄) δ 174.3, 57.1, 53.7, 52.4, 41.0, 35.3, 31.1, 20.4 ppm.

LC/MS (m/z): [M+H]⁺ 175.2.

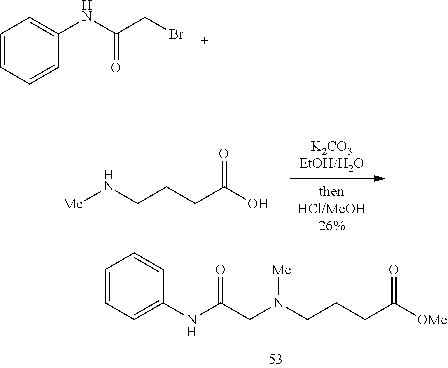 [SMILES: O=C(CBr)Nc1ccccc1 ; CNCCCC(=O)O ; "K2CO3EtOH/H2O" ; "thenHCl/MeOH26%" ; COC(=O)CCCN(C)CC(=O)Nc1ccccc1]

Methyl 4-(methyl(2-oxo-2-(phenylamino)ethyl)amino)butanoate (53): 2-bromo-N-phenylacetamide (0.600 g, 2.80 mmol, 1.0 equiv), N-Me-gamma-aminobutyric acid-1C1 (0.450 g, 2.94 mmol, 1.05 equiv) and K₂CO₃ (0.775 g, 5.61 mmol, 2.0 equiv) were suspended in DMF (10 mL) and stirred at 100° C. for 1 h, then acidified with 2 M HCl and evaporated to dryness. The residue was resuspended in MeOH (15 mL), acidified with HCl (2 M in MeOH, 0.5 mL), stirred at rt over night, then concentrated. The residue was dissolved in dilute K₂CO₃ solution (25 mL) and extracted with CH₂Cl₂ (3×20 mL), dried (MgSO₄) and concentrated in vacuo. The crude product was purified by MPLC (24 g silica, gradient: 0→10% MeOH in CH₂Cl₂ over 20 CV) to provide 53 as a colorless oil (0.192 g, 0.726 mmol, 26% yield).

TLC R_{f} 0.69 (10% MeOH in CH₂Cl₂).

¹H NMR (600 MHz, CDCl₃) δ 9.07 (s, 1H), 7.66-7.62 (m, 2H), 7.36-7.30 (m, 2H), 7.12-7.07 (m, 1H), 3.66 (s, 3H), 3.13 (s, 2H), 2.53 (t, J=7.0 Hz, 2H), 2.40 (t, J=7.0 Hz, 2H), 2.33 (s, 3H), 1.88 (p, J=7.0 Hz, 2H) ppm.

LC/MS (m/z): [M+H]⁺ 265.2.

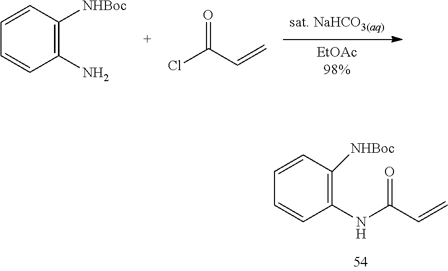

tert-Butyl (2-acrylamidophenyl)carbamate (54): To N-Boc-1,2-phenylenediamine (500 mg, 2.40 mmol, 1.0 equiv) vigorously stirred in sat. NaHCO₃ solution (10 mL) and EtOAc (10 mL) was added acryloyl chloride (217 μL, 2.64 mmol, 1.1 equiv). After stirring for 5 min, the reaction mixture was diluted with EtOAc (20 mL), phases were separated, and the organic layer was washed with sat. NaHCO₃ solution (2×20 mL) and water (20 mL), then dried (MgSO₄) and concentrated to provide pure 54 as an off-white solid (619 mg, 2.36 mmol, 98% yield).

TLC R_{f} 0.24 (30% EtOAc in hexane).

¹H NMR (600 MHz, CDCl3) δ 8.46 (s, 1H), 7.57-7.52 (m, 1H), 7.34-7.29 (m, 1H), 7.18-7.10 (m, 2H), 6.92 (s, 1H), 6.40 (d, J=17.0 Hz, 1H), 6.24 (dd, J=17.0, 10.4 Hz, 1H), 5.76 (d, J=10.4 Hz, 1H), 1.51 (s, 9H) ppm.

LC/MS (m/z): [M+Na]⁺285.1.

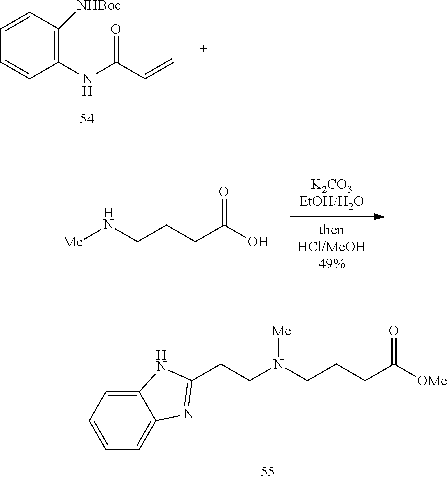 [SMILES: C=CC(=O)Nc1ccccc1NC(=O)OC(C)(C)C ; CNCCCC(=O)O ; "K2CO3EtOH/H2O" ; "thenHCl/MeOH49%" ; COC(=O)CCCN(C)CCc1nc2ccccc2[nH]1]

Methyl 4-((2-(benzimidazol-2-yl)ethyl)(methyl)amino)butanoate (55): 54 (0.609 g, 2.32 mmol, 1.0 equiv), N-Me-gamma-aminobutyric acid.HCl (0.373 g, 2.44 mmol, 1.05 equiv) and K₂CO₃ (0.642 g, 4.64 mmol, 2.0 equiv) were dissolved in water/EtOH (1:1, 10 mL) and stirred at gentle reflux for 16 h, then cooled to rt, diluted with 2 M HCl (10 mL) and evaporated to dryness. Residual water was removed by coevaporation with MeOH and toluene (2×20 mL each), then HCl (0.5 M in dry MeOH, 30 mL) was added and refluxed over night. The reaction mixture was concentrated, dissolved in dilute K₂CO₃ solution (30 mL) and extracted with CH₂Cl₂ (3×25 mL), dried (MgSO₄) and concentrated in vacuo. The crude product was purified by MPLC (24 g silica, gradient: 0→8% MeOH in CH₂Cl₂ over 8 CV, then 8% MeOH over 10 CV) to provide 55 as a brown oil (0.312 g, 1.133 mmol, 49% yield).

TLC R_{f} 0.28 (20% MeOH in CH₂Cl₂).

¹H NMR (600 MHz, CDCl₃) δ 7.59-7.51 (m, 2H), 7.21-7.15 (m, 2H), 3.65 (s, 3H), 3.08 (t, J=6.2 Hz, 2H), 2.78 (t, J=6.2 Hz, 2H), 2.47 (t, J=7.1 Hz, 2H), 2.37 (t, J=7.1 Hz, 2H), 2.29 (s, 3H), 1.87 (app p, J=7.1 Hz, 2H) ppm.

LC/MS (m/z): [M+H]⁺ 276.2.

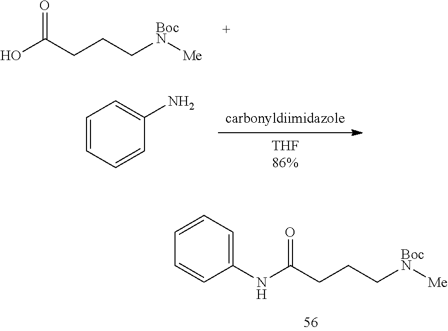 [SMILES: CN(CCCC(=O)O)C(=O)OC(C)(C)C ; Nc1ccccc1 ; "THF86%" ; "carbonyldiimidazole" ; CN(CCCC(=O)Nc1ccccc1)C(=O)OC(C)(C)C]

tert-Butyl methyl(4-oxo-4-(phenylamino)butyl)carbamate (56): To a stirred suspension of carbonyldiimidazole (0.616 g, 3.80 mmol, 1.1 equiv) in THF (5 mL) was added N-Me-N-Boc-gamma-aminobutyric acid (0.750 g, 3.45 mmol, 1.0 equiv) and after 3 h of stirring at rt, aniline (0.299 mL, 3.28 mmol, 0.95 equiv) was added and the reaction mixture stirred for 5 h at rt, then diluted with EtOAc (50 mL) and poured into water (200 mL). Phases were separated and the aqueous phase extracted with EtOAc (2×50 mL). Combined organic layers were washed with ice cold 0.1 M HCl (2×100 mL), sat. Na₂CO₃ solution (100 mL), then dried (MgSO₄), filtered and concentrated in vacuo to provide 56 as yellow oil (0.821 g, 2.81 mmol, 86% yield).

TLC R_{f} 0.47 (10% MeOH in CH₂Cl₂).

¹H NMR (600 MHz, CDCl₃) δ 9.37 (br s, 1H), 7.64 (br s, 2H), 7.31 (t, J=7.8 Hz, 2H), 7.07 (t, J=7.5 Hz, 1H), 3.36 (br s, 2H), 2.86 (s, 3H), 2.31 (t, J=6.3 Hz, 2H), 1.91 (app p, J=6.5 Hz, 2H), 1.49 (s, 9H) ppm.

LC/MS (m/z): [M+Na]⁺315.2.

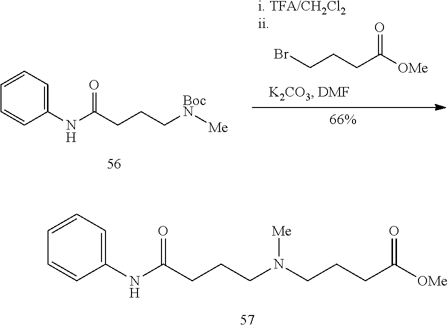 [SMILES: "i. TFA/CH2Cl2ii.   K2CO3, DMF" ; COC(=O)CCCBr ; CN(CCCC(=O)Nc1ccccc1)C(=O)OC(C)(C)C ; "66%" ; COC(=O)CCCN(C)CCCC(=O)Nc1ccccc1]

Methyl 4-(methyl(4-oxo-4-(phenylamino)butyl)amino)butanoate (57): To a stirred solution of 56 (0.810 g, 2.77 mmol, 1.0 equiv) in CH₂Cl₂ (8 mL) was added TFA (2.1 mL, 27.7 mmol, 10 equiv), stirred for 1 h, then concentrated to dryness. The residue was dissolved in DMF (5 mL), K₂CO₃ (1.15 g, 8.31 mmol, 3.0 equiv) and methyl 4-bromobutyrate (0.367 mL, 2.91 mmol, 1.05 equiv) were added. The reaction mixture was heated to 100° C. and stirred for 4.5 h, then concentrated and dissolved in dilute K₂CO₃ solution (100 mL), extracted with CH₂Cl₂ (4×60 mL) and the combined organic layers were washed with brine (100 mL, basified to pH 12), then dried (MgSO₄) and concentrated in vacuo. The crude product was purified by FCC (78 g silica, eluent: 5%, then 10% MeOH and 0.5% NH₄OH in CH₂Cl₂) to provide 57 as yellow oil (0.537 g, 1.84 mmol, 66% yield).

TLC R_{f} 0.17 (10% MeOH and 0.5% NH₄OH in CH₂Cl₂).

¹H NMR (600 MHz, CDCl₃) δ 9.15 (s, 1H), 7.57-7.52 (m, 2H), 7.29-7.23 (m, 2H), 7.03 (tt, J=7.3, 1.2 Hz, 1H), 3.64 (s, 3H), 2.44-2.37 (m, 6H), 2.32 (t, J=7.1 Hz, 2H), 2.21 (s, 3H), 1.87-1.77 (m, 4H) ppm.

LC/MS (m/z): [M+H]⁺ 293.2.

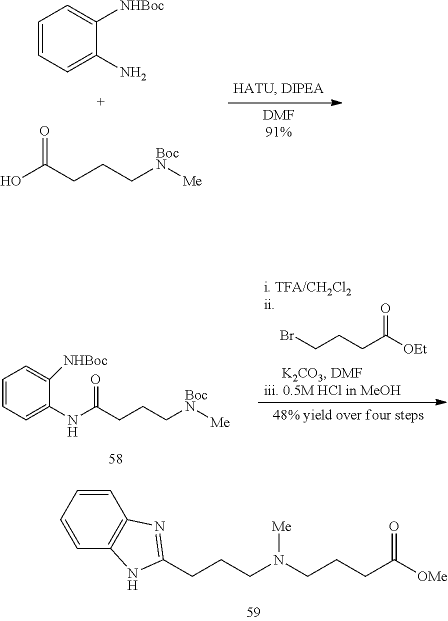

Methyl 4-((3-(benzimidazol-2-yl)propyl)(methyl)amino)butanoate (59): N-Boc-1,2-phenylenediamine (0.200 g, 0.960 mmol, 1.0 equiv), N-Me-N-Boc-gamma-aminobutyric acid (0.209 g, 0.960 mmol, 1.0 equiv) and HATU (0.438 g, 1.15 mmol, 1.2 equiv) were dissolved in DMF (2.5 mL), DIPEA (0.384 mL, 2.88 mmol, 3.0 equiv) was added and the reaction mixture was heated by μwave irradiation to 80° C. for 20 min. The reaction mixture was diluted with EtOAc (50 mL), washed with dilute K₂CO₃ solution (30 mL) and brine (2×30 mL), dried (MgSO₄) and concentrated in vacuo. The crude product was purified by MPLC (10 g silica, gradient: EtOAc in hexane) to provide 58 as a white foam (355 mg, 0.870 mmol, 91% yield, m/z: [M+Na]⁺: 430.2, TLC R_{f} 0.57 60% EtOAc in hexane), which was dissolved in TFA/CH₂Cl₂ (25% TFA in CH₂Cl₂, 10 mL) and stirred at rt for 90 min, then concentrated to dryness. The residue was dissolved in DMF (2.0 mL), ethyl 4-bromobyturate (131 μL, 0.914 mmol, 1.05 equiv) and K₂CO₃ (0.481 g, 3.48 mmol, 4.0 equiv) were added and the mixture stirred at 85° C. for 18 h, then acidified with 1 M aqueous HCl and the solution concentrated to dryness. The residue was refluxed in methanolic HCl (0.5 M, 30 mL) for 21 h, then concentrated, redissolved in water (30 mL), adjusted to pH 12 with K₂CO₃, extracted with CH₂Cl₂ (4×30 mL), dried (MgSO₄) and concentrated in vacuo. The crude product was purified by MPLC (12 g silica, gradient: 0→20% MeOH in CH₂Cl₂) to provide 59 as a brown oil, which solidified over time (0.133 g, 0.460 mmol, 48% yield over four steps).

TLC R_{f} 0.19 (20% MeOH in CH₂Cl₂).

¹H NMR (600 MHz, CDCl₃) δ 7.57-7.52 (m, 2H), 7.20-7.17 (m, 2H), 3.68 (s, 3H), 3.07-3.02 (m, 2H), 2.49 (t, J=5.9 Hz, 2H), 2.45 (d, J=7.2 Hz, 2H), 2.39 (t, J=7.2 Hz, 2H), 2.26 (s, 3H), 1.98 (app p, J=6.2 Hz, 2H), 1.89 (app p, J=7.2 Hz, 2H) ppm.

LC/MS (m/z): [M+H]⁺ 290.2.

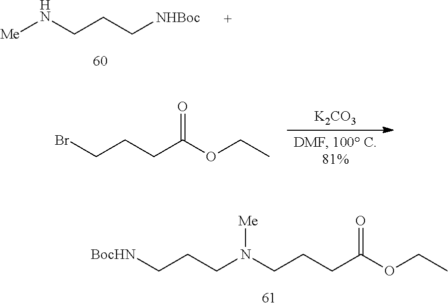

Ethyl 4-((3-((tert-butoxycarbonyl)amino)propyl)(methyl)amino)butanoate (61): To a stirred suspension of 60 (2.50 g, 13.28 mmol, 1.0 equiv) and K₂CO₃ (3.67 g, 26.56 mmol, 2.0 equiv) in DMF (10 mL) was added ethyl 4-bromobutyrate (2.09 mL, 14.61 mmol, 1.1 equiv). The reaction mixture was stirred at 100° C. for 16 h, then cooled to rt and poured into water (180 mL) and extracted with EtOAc (3×75 mL). Combined organic layers were dried (MgSO₄), filtered and concentrated in vacuo. The crude product was purified by FCC (500 g silica, eluent: 8%, then 12%, then 20% MeOH in CH₂Cl₂) and combined product fractions were filtered through a pad of celite to provide 61 as a clear, yellow oil (3.27 g, 10.82 mmol, 81% yield).

TLC R_{f} 0.23 (10% MeOH in CH₂Cl₂).

¹H NMR (400 MHz, CDCl₃) δ 5.33 (br s, 1H), 4.12 (q, J=7.1 Hz, 2H), 3.19 (app q, J=6.3 Hz, 2H), 2.50 (br s, 4H), 2.35 (t, J=7.3 Hz, 2H), 2.30 (br s, 3H), 1.91-1.79 (m, 2H), 1.78-1.66 (m, 2H), 1.43 (s, 9H), 1.25 (t, J=7.1 Hz, 3H) ppm.

LC/MS (m/z): [M+H]⁺ 303.0.

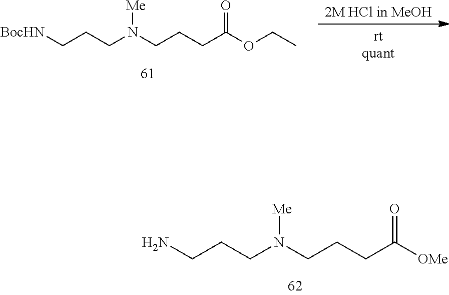 [SMILES: CCOC(=O)CCCN(C)CCCNC(=O)OC(C)(C)C ; "2M HCl in MeOH" ; "rtquant" ; COC(=O)CCCN(C)CCCN]

Methyl 4-((3-aminopropyl)(methyl)amino)butanoate (62): 61 (3.27 g, 10.82 mmol, 1.0 equiv) was dissolved in 2 M HCl in MeOH (135 mL, 270.4 mmol, 25 equiv) and stirred at rt for 26 h. The solvent was removed in vacuo and residual acid was removed by coevaporation with MeOH (3×50 mL) and high vacuum to provide the 21-101 salt of 62 as a brown-orange amorphous solid (2.924 g, 11.20 mmol, quant yield). The product was stored as a stock solution in MeOH and used without further purification.

¹H NMR (400 MHz, MeOD-d₄) δ 3.70 (s, 3H), 3.44-3.14 (m, 4H, partly overlapped with MeOD signal), 3.08 (t, J=7.6 Hz, 2H), 2.93 (s, 3H), 2.52 (t, J=7.0 Hz, 2H), 2.18 (app p, J=7.7 Hz, 2H), 2.13-2.02 (m, 2H) ppm.

LC/MS (m/z): [M+H]⁺ 189.1.

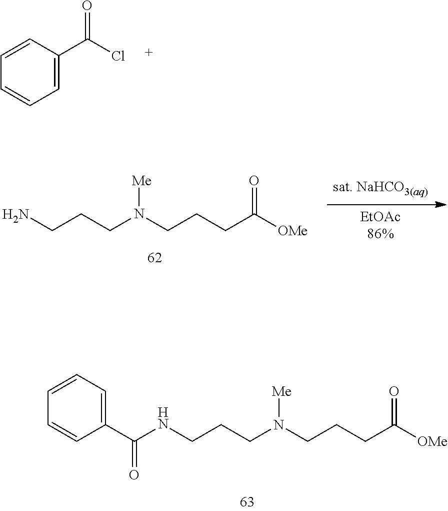 [SMILES: O=C(Cl)c1ccccc1 ; COC(=O)CCCN(C)CCCN ; "EtOAc86%" ; "sat. NaHCO3(aq)" ; COC(=O)CCCN(C)CCCNC(=O)c1ccccc1]

Methyl 4-((3-benzamidopropyl)(methyl)amino)butanoate (63): To 62 2.HCl (182.8 mg, 0.700 mmol, 1.0 equiv) vigorously stirred in sat. NaHCO₃ solution (4 mL) and EtOAc (4 mL) was added benzoyl chloride (0.177 mL, 1.54 mmol, 2.2 equiv). After stirring for 90 min, the reaction mixture was diluted with EtOAc (15 mL) and sat. NaHCO₃ solution (15 mL), phases were separated, and the aqueous layer was extracted with EtOAc (2×20 mL), combined organic layers were dried (MgSO₄) and concentrated. The crude product was purified by MPLC (12 g silica, gradient: 0→8% for 12 CV, then 8% MeOH in CH₂Cl₂ for 12 CV) to provide sufficiently pure 63 as a yellow oil (175.2 mg, approx. 0.599 mmol, approx. 86% yield).

TLC R_{f} 0.20 (10% MeOH in CH₂Cl₂).

¹H NMR (400 MHz, CDCl₃) δ 8.10-8.01 (m, 1H), 7.87-7.80 (m, 2H), 7.51-7.33 (m, 4H, impurities), 6.67 (s, 1H), 3.63 (s, 3H), 3.58-3.52 (m, 2H), 2.66 (t, J=6.3 Hz, 2H), 2.58-2.50 (m, 2H), 2.36 (s, 3H), 2.32 (t, J=7.2 Hz, 2H), 1.92-1.80 (m, 4H) ppm.

LC/MS (m/z): [M+H]⁺ 292.9.

Note: The compound was obtained with less than 95% purity. Benzoyl chloride was the main impurity, but the material was sufficiently pure for the next reaction and was used without further purification.

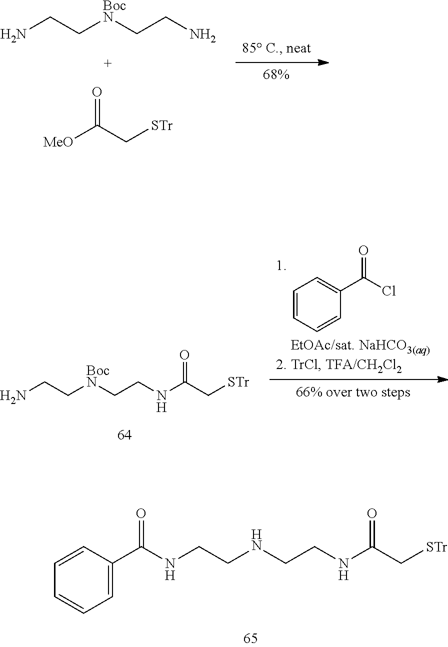

N-(2-((2-(2-(Tritylthio)acetamido)ethyl)amino)ethyl)benzamide (65): methyl 2-(tritylthio)acetate⁴⁷ (0.628 g, 1.804 mmol, 1.0 equiv) was added in small portions to neat tert-butyl bis(2-aminoethyl)carbamate⁴⁸ (0.734 g, 3.608 mmol, 2.0 equiv) heated to 85° C. in an open flask and stirred for 3.5, then cooled to rt, dissolved in EtOAc (100 mL) and washed with sat. NaHCO₃ solution (2×80 mL), dried (MgSO₄) and concentrated. The crude product was purified by MPLC (12 g silica, gradient: 0→20% MeOH and 0.5% NH₄OH in CH₂Cl₂ for 20 CV) to provide 64 as a white foam (638 mg, 1.228 mmol, 68% yield, (m/z): [M+H]⁺ 520.3, TLC R_{f} 0.61 20% MeOH and 0.5% NH₄OH in CH₂Cl₂), which was dissolved in sat. NaHCO₃ solution (10 mL) and EtOAc (10 mL). Benzoyl chloride (0.163 mL, 1.412 mmol, 1.15 equiv) was added with vigorous stirring, after 10 min the reaction mixture was diluted with sat. NaHCO₃ solution (40 mL) and EtOAc (40 mL), phases were separated and the organic layer was washed with sat. NaHCO₃ solution (40 mL), then dried (MgSO₄) and concentrated. The obtained white foam (787 mg) was dissolved in CH₂Cl₂ (10 mL), trityl chloride (0.103 mg, 0.368 mmol, 0.3 equiv) and TFA (5.0 mL, 65.1 mmol, 53 equiv) were added and stirred at rt for 2 h. Then, the reaction was diluted with CH₂Cl₂ (80 mL), cooled to 0° C. and quenched with 1 M NaOH (100 mL). Phases were separated and the aqueous phase extracted with CH₂Cl₂ (4×50 mL). The combined organic layers were dried (MgSO₄) and concentrated. The crude product was purified by MPLC (12 g silica, gradient: 0→10% for 10 CV, then 10% MeOH and 0.5% NH₄OH in CH₂Cl₂ for 10 CV) to provide sufficiently pure (approx. 80%) 65 as a white foam (528 mg, approx. 0.806 mmol, approx. 49% yield over three steps).

TLC R_{f} 0.41 (10% MeOH and 0.5% NH₄OH in CH₂Cl₂).

¹H NMR (400 MHz, CDCl₃) δ 7.41-7.35 (m, 8H), 7.31-7.18 (m, overlapped with CDCl₃ peak), 6.84-6.72 (br, 1H), 6.32 (t, J=5.5 Hz, 1H), 3.50 (app q, J=5.4 Hz, 2H), 3.10 (s, 2H), 3.06 (app q, J=5.9 Hz, 2H), 2.86-2.79 (m, 2H), 2.65-2.58 (m, 2H), 1.77 (s, 3H) ppm. Impurity signals are not listed.

LC/MS (m/z): [M+H]⁺ 524.2. Purity by evaporative light scattering: approx. 80% by weight.

Note: The compound was obtained with approx. 80% purity, but the material was sufficiently pure for the next reaction and was used without further purification. The impurity gave an (m/z) of 243.2 (Tr⁺) and was removed in the subsequent steps. Yields are corrected for purity.

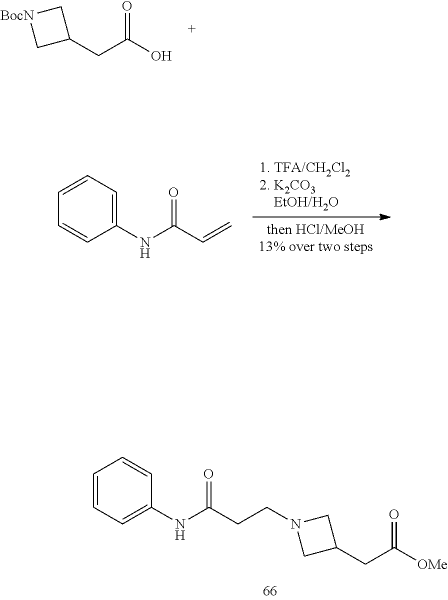 [SMILES: CC(C)(C)OC(=O)N1CC(CC(=O)O)C1 ; "1. TFA/CH2Cl22. K2CO3    EtOH/H2O" ; C=CC(=O)Nc1ccccc1 ; "then HCl/MeOH13% over two steps" ; COC(=O)CC1CN(CCC(=O)Nc2ccccc2)C1]

Methyl 2-(1-(3-oxo-3-(phenylamino)propyl)azetidin-3-yl)acetate (66): 2-(1-(tert-butoxycarbonyl)azetidin-3-yl)acetic acid (1.000 g, 4.646 mmol, 1.0 equiv) was dissolved in TFA (25% in CH₂Cl₂, 25 mL, 65.04 mmol, 14 equiv) at 0° C., allowed to warm to rt while stirring for 1 h, then concentrated to dryness. Excess acid was removed by lyophilization from water. The residue was dissolved in water/EtOH (1:1, 15 mL), together with N-phenylacrylamide (0.743 g, 5.05 mmol, 1.05 equiv) and K₂CO₃ (1.994 g, 14.43 mmol, 3.0 equiv). The reaction mixture was stirred at 80° C. for 5 h then cooled to rt, diluted with 1 M HCl (40 mL), extracted with EtOAc (2×30 mL) and the aqueous layer evaporated to dryness. The residue was suspended in MeOH (25 mL), acidified with HCl in MeOH, stirred at rt for 3 h, then concentrated to dryness. The residue was dissolved in dilute K₂CO₃ solution (40 mL) and extracted with CH₂Cl₂ (3×25 mL), dried (MgSO₄) and concentrated in vacuo. The crude product was purified by MPLC (40 g silica, gradient: CH₂Cl₂ for 2 CV, 0→7% MeOH over 8 CV, then 7% MeOH in CH₂Cl₂ over 7 CV) to provide sufficiently pure 66 as a yellow oil (0.1729 g, crude, approx. 13% yield over two steps). The material contained non-ester impurities but was used without further purification.

TLC R_{f} 0.50 (20% MeOH in CH₂Cl₂).

LC/MS (m/z): [M+H]⁺ 277.2.

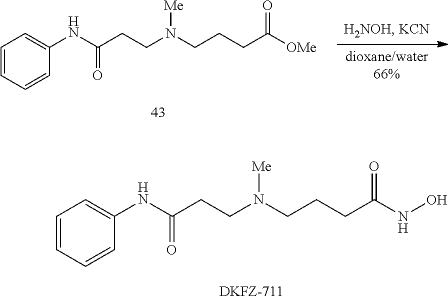

N-(2-((4-(Hydroxyamino)-4-oxobutyl)(methyl)amino)ethyl)benzamide (DKFZ-711): The title compound was prepared from ester 43 (187.3 mg, 0.673 mmol, 1.0 equiv) according to General Procedure A, and purified by HPLC Acidic Method (gradient: 1→10% B in 3 min, then 10→45% B in 11 min) to provide the TFA salt of DKFZ-711 as an orange, hygroscopic, amorphous solid (174.2 mg, 0.443 mmol, 66% yield).

TLC R_{f} 0.21 (10% MeOH and 0.5% NH₄OH in CH₂Cl₂).

¹H NMR (400 MHz, D₂O) δ 7.49-7.40 (m, 4H), 7.33-7.22 (m, 1H), 3.70-3.58 (m, 1H), 3.51-3.37 (m, 1H), 3.33-3.18 (m, 2H), 2.97 (t, J=6.7 Hz, 2H), 2.93 (s, 3H), 2.32 (t, J=7.2 Hz, 2H), 2.14-2.03 (m, 2H) ppm.

¹³C NMR (101 MHz, D₂O) δ 171.1, 170.1, 136.5, 129.2, 125.7, 121.8, 55.5, 51.8, 40.0, 30.0, 29.0, 19.6 ppm. TFA signals not listed.

HR-MS (m/z): [M+H]⁺ calcd for C₁₄H₂₂N₃O₃⁺: 280.1656; found: 280.1656.

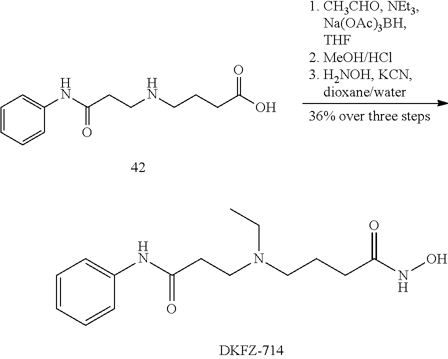 [SMILES: "1. CH3CHO, NEt3,    Na(OAc)3BH,    THF2. MeOH/HCl3. H2NOH, KCN,    dioxane/water" ; O=C(O)CCCNCCC(=O)Nc1ccccc1 ; "36% over three steps" ; CCN(CCCC(=O)NO)CCC(=O)Nc1ccccc1]

4-(Ethyl(3-oxo-3-(phenylamino)propyl)amino)-N-hydroxybutanamide (DKFZ-714): The corresponding methyl ester of the title compound was prepared from 42.HCl (100 mg, 0.349 mmol, 1.0 equiv) according to General Procedure C. The crude product was converted to the hydroxamic acid according to General Procedure A, and purified by HPLC Acidic Method (gradient: 1→10% B in 3 min, then 10→45% B in 11 min) to provide the TFA salt of DKFZ-714 as a yellow, hygroscopic, amorphous solid (51.8 mg, 0.127 mmol, 36% yield over three steps).

TLC R_{f} 0.08 (10% MeOH in CH₂Cl₂).

¹H NMR (400 MHz, D₂O) δ 7.48-7.41 (m, 4H), 7.32-7.22 (m, 1H), 3.59-3.50 (m, 2H), 3.31 (q, J=7.3 Hz, 2H), 3.28-3.18 (m, 2H), 2.95 (t, J=6.8 Hz, 2H), 2.36-2.28 (m, 2H), 2.12-2.01 (m, 2H), 1.34 (t, J=7.3 Hz, 3H) ppm.

¹³C NMR (101 MHz, D₂O) δ 174.0, 173.0, 139.3, 132.1, 128.6, 124.7, 54.7, 51.3, 51.1, 32.9, 31.9, 22.1, 11.0 ppm. TFA signals are not listed.

HR-MS (m/z): [M+H]⁺ calcd for C₁₅H₂₄N₃O₃⁺: 294.1812; found: 294.1816.

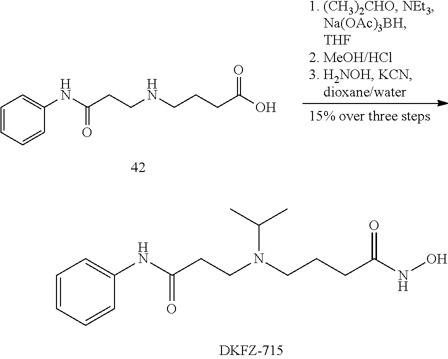

4-(Isopropyl(3-oxo-3-(phenylamino)propyl)amino)-N-hydroxybutanamide (DKFZ-715): The corresponding methyl ester of the title compound was prepared from 42.HCl (150 mg, 0.523 mmol, 1.0 equiv) according to General Procedure C. The crude product was converted to the hydroxamic acid according to General Procedure A, and purified by HPLC Acidic Method (gradient: 1→10% B in 3 min, then 10→45% B in 11 min) to provide the TFA salt of DKFZ-715 as a yellow, hygroscopic, amorphous solid (33.0 mg, 0.078 mmol, 15% yield over three steps).

TLC R_{f} 0.04 (10% MeOH in CH₂Cl₂).

¹H NMR (400 MHz, D₂O) δ 7.48-7.39 (m, 4H), 7.28 (dtd, J=8.4, 5.0, 3.1 Hz, 1H), 3.78 (hept, J=6.6 Hz, 1H), 3.59 (dt, J=13.9, 7.0 Hz, 1H), 3.39 (dt, J=13.4, 6.5 Hz, 1H), 3.25 (ddd, J=13.3, 9.0, 6.8 Hz, 1H), 3.14 (ddd, J=13.4, 9.0, 6.5 Hz, 1H), 2.94 (t, J=6.8 Hz, 2H), 2.33 (t, J=7.0 Hz, 2H), 2.14-1.96 (m, 2H), 1.35 (t, J=7.1 Hz, 6H) ppm.

¹³C NMR (101 MHz, D₂O) δ 174.0, 173.1, 139.3, 132.1, 128.6, 124.7, 58.6, 52.7, 48.9, 33.5, 32.1, 23.2, 18.7, 18.3 ppm. TFA signals are not listed.

HR-MS (m/z): [M+H]⁺ calcd for C₁₆H₂₆N₃O₃⁺: 308.1969; found: 308.1974.

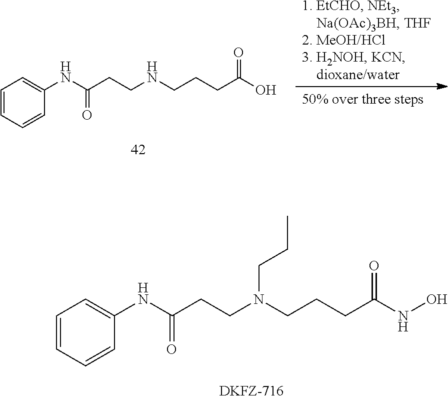 [SMILES: "1. EtCHO, NEt3,    Na(OAc)3BH, THF2. MeOH/HCl3. H2NOH, KCN,    dioxane/water" ; O=C(O)CCCNCCC(=O)Nc1ccccc1 ; "50% over three steps" ; CCCN(CCCC(=O)NO)CCC(=O)Nc1ccccc1]

4-(Propyl(3-oxo-3-(phenylamino)propyl)amino)-N-hydroxybutanamide (DKFZ-716): The corresponding methyl ester of the title compound was prepared from 42.HCl (150 mg, 0.523 mmol, 1.0 equiv) according to General Procedure C. The crude product was converted to the hydroxamic acid according to General Procedure A, and purified by HPLC Acidic Method (gradient: 1→10% B in 3 min, then 10→45% B in 11 min) to provide the TFA salt of DKFZ-716 as a yellow, hygroscopic, amorphous solid (110 mg, 0.260 mmol, 50% yield over three steps).

TLC R_{f} 0.17 (10% MeOH in CH₂Cl₂).

¹H NMR (400 MHz, D₂O) δ 7.48-7.39 (m, 4H), 7.27 (tt, J=5.6, 2.9 Hz, 1H), 3.59-3.48 (m, 2H), 3.20 (dt, J=19.4, 9.8 Hz, 4H), 2.93 (d, J=6.5 Hz, 2H), 2.30 (ddd, J=7.4, 5.2, 2.0 Hz, 2H), 2.10-1.97 (m, 2H), 1.83-1.67 (m, 2H), 0.98 (td, J=7.4, 1.6 Hz, 3H) ppm.

¹³C-DEPT NMR (101 MHz, D₂O) δ 129.3 (CH), 125.8 (CH), 121.8 (CH), 55.0 (CH₂), 52.4 (CH₂), 48.8 (CH₂), 30.0 (CH₂), 29.0 (CH₂), 19.2 (CH₂), 16.9 (CH₂), 10.1 (CH₃) ppm.

HR-MS (m/z): [M+H]⁺ calcd for C₁₆H₂₆N₃O₃⁺: 308.1969; found: 308.1974.

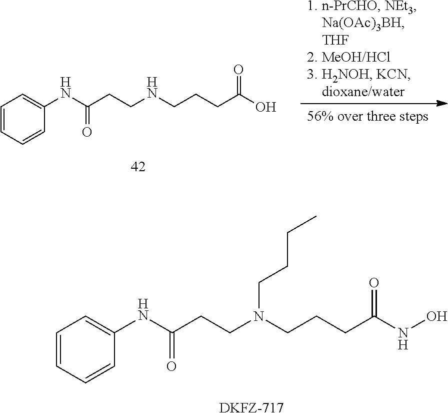

4-(Butyl(3-oxo-3-(phenylamino)propyl)amino)-N-hydroxybutanamide (DKFZ-717): The corresponding methyl ester of the title compound was prepared from 42.HCl (150 mg, 0.523 mmol, 1.0 equiv) according to General Procedure C. The crude product was converted to the hydroxamic acid according to General Procedure A, and purified by HPLC Acidic Method (gradient: 1→10% B in 3 min, then 10→45% B in 11 min) to provide the TFA salt of DKFZ-717 as a yellow, hygroscopic, amorphous solid (127 mg, 0.292 mmol, 56% yield over three steps).

TLC R_{f} 0.08 (10% MeOH in CH₂Cl₂).

¹H NMR (400 MHz, D₂O) δ 7.46-7.40 (m, 4H), 7.30-7.22 (m, 1H), 3.59-3.48 (m, 2H), 3.27-3.14 (m, 4H), 2.98-2.88 (m, 2H), 2.35-2.25 (m, 2H), 2.10-1.98 (m, 2H), 1.77-1.65 (m, 2H), 1.38 (app p, J=7.5 Hz, 2H), 0.97-0.89 (m, 3H) ppm.

¹³C NMR (101 MHz, D₂O) δ 171.2, 170.2, 136.5, 129.3, 125.8, 121.9, 53.3, 52.4, 48.9, 30.0, 29.0, 25.2, 19.3 (2 CH₂), 12.8 ppm. TFA signals are not listed.

HR-MS (m/z): [M+H]⁺ calcd for C₁₇H₂₈N₃O₃⁺: 322.2125; found: 322.2127.

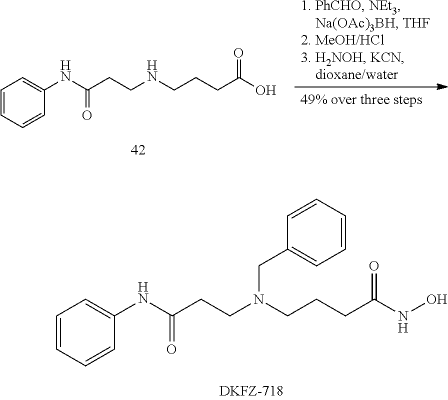 [SMILES: "1. PhCHO, NEt3,    Na(OAc)3BH, THF2. MeOH/HCl3. H2NOH, KCN,    dioxane/water" ; O=C(O)CCCNCCC(=O)Nc1ccccc1 ; "49% over three steps" ; O=C(CCCN(CCC(=O)Nc1ccccc1)Cc1ccccc1)NO]

4-(Benzyl(3-oxo-3-(phenylamino)propyl)amino)-N-hydroxybutanamide (DKFZ-718): The corresponding methyl ester of the title compound was prepared from 42.HCl (150 mg, 0.523 mmol, 1.0 equiv) according to General Procedure C. The crude product was converted to the hydroxamic acid according to General Procedure A, and purified by HPLC Acidic Method (gradient: 1→10% B in 3 min, then 10→45% B in 11 min) to provide the TFA salt of DKFZ-718 as an off-white solid (119 mg, 0.254 mmol, 49% yield over three steps).

TLC R_{f} 0.17 (10% MeOH in CH₂Cl₂).

¹H NMR (400 MHz, MeOD-d₄) δ 7.62-7.48 (m, 7H), 7.35-7.29 (m, 2H), 7.11 (tt, J=7.4, 1.2 Hz, 1H), 4.45 (s, 2H), 3.54 (t, J=6.8 Hz, 2H), 3.28 (t, J=7.4 Hz, 2H), 2.91 (t, J=6.8 Hz, 2H), 2.27 (t, J=6.3 Hz, 2H), 2.09 (p, J=6.7 Hz, 2H) ppm.

¹³C NMR (101 MHz, MeOD-d₄) δ 171.4, 170.1, 139.4, 132.1, 131.3, 130.8, 130.6, 129.9, 125.5, 121.3, 58.9, 54.6, 50.3, 31.1, 30.6, 20.7 ppm. TFA signals are not listed.

HR-MS (m/z): [M+H]⁺ calcd for C₂₀H₂₆N₃O₃⁺: 356.1969; found: 356.1972.

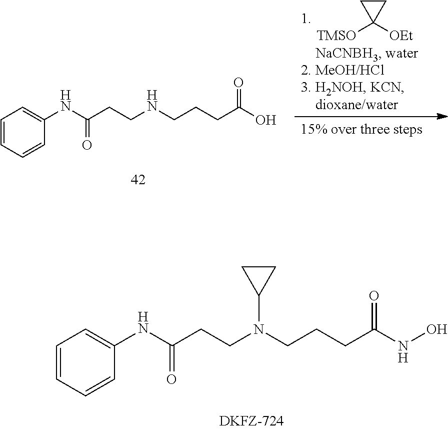

4-(Cyclopropyl(3-oxo-3-(phenylamino)propyl)amino)-N-hydroxybutanamide (DKFZ-724): To a Schlenk tube charged with 42.HCl (150 mg, 0.523 mmol, 1.0 equiv) and NaCNBH₃ (49.3 mg, 0.785 mmol, 1.5 equiv), degassed water (1.5 mL), (1-ethoxycyclopropoxy)trimethylsilane (1.05 mL, 5.23 mmol, 10 equiv) and HCl (37%, 51.5 μL, 0.523 mmol, 1.0 equiv) were added under argon atmosphere. The mixture was stirred vigorously for 7 d and more NaCNBH₃ (2.2 equiv) was added in small portions as a solution in degassed water until complete consumption of starting material. The reaction was stopped by evaporating to dryness, esterified and the crude product isolated as described in General Procedure C. The crude product was converted to the hydroxamic acid according to General Procedure A, and purified by HPLC Acidic Method (gradient: 1→10% B in 3 min, then 10→45% B in 11 min) to provide the TFA salt of DKFZ-724 as a white solid (32.8 mg, 0.078 mmol, 15% yield over three steps).

TLC R_{f} 0.17 (10% MeOH in CH₂Cl₂).

¹H NMR (400 MHz, D₂O) δ 7.50-7.40 (m, 4H), 7.29 (qt, J=5.5, 2.9 Hz, 1H), 3.71 (t, J=6.7 Hz, 2H), 3.42-3.31 (m, 2H), 3.04 (t, J=6.7 Hz, 2H), 2.86 (ddd, J=11.2, 7.2, 4.6 Hz, 1H), 2.33 (t, J=7.1 Hz, 2H), 2.15 (dq, J=14.6, 7.2 Hz, 2H), 1.06 (dd, J=13.8, 3.2 Hz, 2H), 1.05 (s, 2H) ppm.

¹³C NMR (101 MHz, D₂O) δ 174.1, 173.1, 139.3, 132.1, 128.6, 124.7, 58.1, 54.0, 40.4, 33.3, 32.0, 22.3, 7.3 (2 CH₂) ppm. TFA signals are not listed.

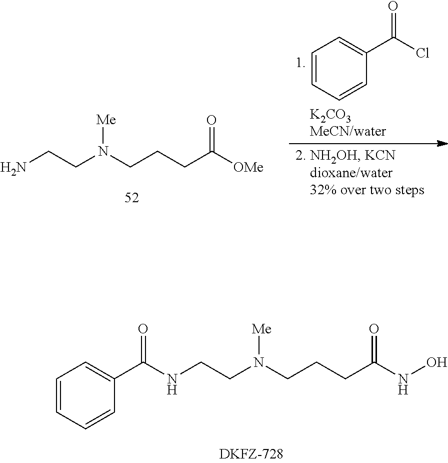

N-(2-((4-(Hydroxyamino)-4-oxobutyl)(methyl)amino)ethyl)benzamide (DKFZ-728): To a stirred solution of 52 2.HCl (321 mg, 1.30 mmol, 1.0 equiv) and K₂CO₃ (629 mg, 4.55 mmol, 3.5 equiv) in 12 mL MeCN/water (10:2) was added BzCl (172 μL, 1.50 mmol, 1.15 equiv) and stirred at rt until TLC indicated complete conversion, then concentrated in vacuo. The residue was dissolved in EtOAc (25 mL) and dilute K₂CO₃ solution (30 mL), phases were separated and the aqueous phase extracted with EtOAc (2×25 mL). Combined organic layers were washed with dilute K₂CO₃ solution (2×25 mL) and brine (25 mL), then dried (MgSO₄) and concentrated. The crude product was purified by MPLC (24 g silica, gradient: 0→10% MeOH in CH₂Cl₂) to provide the corresponding methyl ester of DKFZ-728 (167 mg, m/z: [M+H]⁺: 279.2), which was directly converted to DKFZ-728 according to General Procedure A, and purified by RP-MPLC (43 g C18 silica, gradient: 0% B over 3 CV, then 0→20% over 12 CV) to provide DKFZ-728 as a white solid (115 mg, 0.412 mmol, 32% yield over two steps).

TLC R_{f} 0.26 (20% MeOH in CH₂Cl₂)

¹H NMR (600 MHz, DMSO-d₆) δ 10.34 (s, 1H), 8.68 (s, 1H), 8.38 (t, J=5.7 Hz, 1H), 7.84-7.80 (m, 2H), 7.53-7.49 (m, 1H), 7.47-7.43 (m, 2H), 3.39-3.31 (m, overlapped with water peak), 2.47 (t, J=7.0 Hz, 2H), 2.32 (t, J=7.2 Hz, 2H), 2.19 (s, 3H), 1.96 (t, J=7.4 Hz, 2H), 1.62 (app p, J=7.3 Hz, 2H) ppm.

¹³C NMR (151 MHz, DMSO-d₆) δ 22.9, 30.1, 37.2, 41.9, 56.1, 56.5, 127.1, 128.3, 131.0, 134.6, 166.1, 169.1 ppm.

LC/MS (m/z): [M+H]⁺ 280.2, [M−H]⁻ 278.2.

HR-MS (m/z): [M+H]⁺ calcd for C₁₄H₂₂N₃O₃⁺: 280.1656; found: 280.1657.

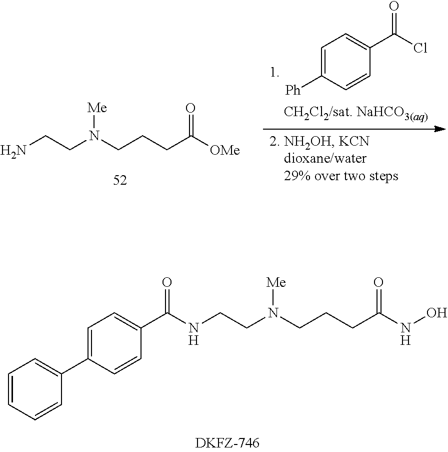

N-(2-((4-(Hydroxyamino)-4-oxobutyl)(methyl)amino)ethyl)-[1,1′-biphenyl]-4-carboxamide (DKFZ-746): To a vigorously stirred solution of 52 2.HCl (82 mg, 0.330 mmol, 1.0 equiv) in sat. NaHCO₃ solution (6 mL) and CH₂Cl₂ (10 mL) was added 4-Phenylbenzoyl chloride (125 mg, 0.577 mmol, 1.8 equiv). The reaction mixture was diluted with water (100 mL) and extracted with CH₂Cl₂ (3×30 mL), combined organic layers were washed with dilute K₂CO₃ solution (twice) and brine (50 mL), then dried (MgSO₄) and concentrated. The residue was directly converted to the hydroxamic acid according to General Procedure A, and purified by HPLC Basic Method (gradient: 1→75% B in 12 min) to provide DKFZ-746 as a white fluffy solid (34.3 mg, 0.097 mmol, 29% yield over two steps).

¹H NMR (400 MHz, MeOD-d₄) δ 7.94-7.87 (m, 2H), 7.73-7.68 (m, 2H), 7.68-7.62 (m, 2H), 7.49-7.42 (m, 2H), 7.40-7.33 (m, 1H), 3.53 (t, J=6.8 Hz, 2H), 2.63 (t, J=6.8 Hz, 2H), 2.50-2.43 (m, 2H), 2.32 (s, 3H), 2.12 (t, J=7.3 Hz, 2H), 1.81 (app p, J=7.3 Hz, 2H) ppm.

¹³C NMR (101 MHz, MeOD-d₄) δ 172.1, 169.9, 145.7, 141.3, 134.3, 130.0, 129.1, 128.9, 128.1, 128.0, 57.9, 57.3, 42.4, 38.5, 31.6, 24.1 ppm.

HR-MS (m/z): [M+H]⁺ calcd for C₂₀H₂₆N₃O₃⁺: 356.1969; found: 356.1972.

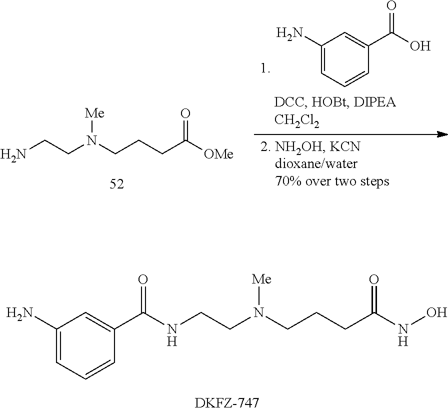 [SMILES: "1.    DCC, HOBt, DIPEA    CH2Cl2" ; Nc1cccc(C(=O)O)c1 ; COC(=O)CCCN(C)CCN ; "2. NH2OH, KCN    dioxane/water    70% over two steps" ; CN(CCCC(=O)NO)CCNC(=O)c1cccc(N)c1]

3-Amino-N-(2-((4-(hydroxyamino)-4-oxobutyl)(methyl)amino)ethyl)benzamide (DKFZ-747): The title compound was prepared from ester 52 2.HCl (80.0 mg, 0.324 mmol, 1.0 equiv) according to General Procedure B, but with DCC as coupling reagent. Precipitating DCU was removed by filtration and the concentrated filtrate was directly converted to the hydroxamic acid according to General Procedure A, and purified by HPLC Basic Method (gradient: 1→30% B in 12 min) to provide DKFZ-747 as an off-white powder (66.8 mg, 0.227 mmol, 70% yield over two steps).

¹H NMR (400 MHz, DMSO-d₆) δ 10.82-8.38 (br, 2H), 8.08 (t, J=5.7 Hz, 1H), 7.05 (t, J=7.8 Hz, 1H), 7.00 (t, J=2.0 Hz, 1H), 6.91 (dt, J=7.6, 1.4 Hz, 1H), 6.70-6.63 (m, 1H), 5.20 (s, 2H), 3.32-3.24 (m, overlapped with water peak), 2.43 (t, J=7.0 Hz, 2H), 2.30 (t, J=7.2 Hz, 2H), 2.17 (s, 3H), 1.96 (t, J=7.4 Hz, 2H), 1.61 (app p, J=7.4 Hz, 2H) ppm.

¹³C NMR (101 MHz, DMSO-d₆) δ 169.1, 166.9, 148.6, 135.6, 128.6, 116.3, 114.2, 112.7, 56.5, 56.2, 41.9, 37.1, 30.1, 22.9 ppm.

HR-MS (m/z): [M+H]⁺ calcd for C₁₄H₂₃N₄O₃⁺: 295.1765; found: 295.1768.

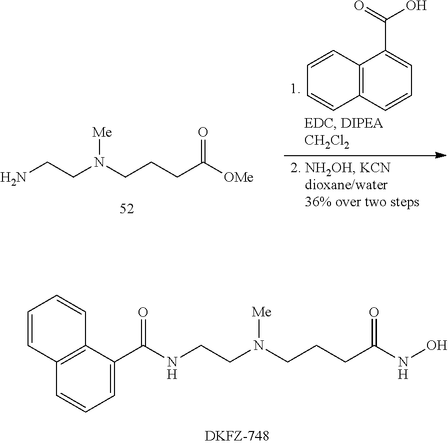

N-(2-((4-(Hydroxyamino)-4-oxobutyl)(methyl)amino)ethyl)-1-naphthamide (DKFZ-748): The title compound was prepared from ester 52 2.HCl (204.1 mg, 0.826 mmol, 1.0 equiv) according to General Procedure B using recrystallized 1-naphthoic acid. The crude product was purified by MPLC (12 g silica, gradient: 0.5% NH₄OH in CH₂Cl₂ for 2 CV, then 0→10% MeOH over 7 CV) to provide the corresponding methyl ester of DKFZ-748 (172 mg, m/z: [M+H]⁺: 329.2), which was directly converted to DKFZ-748 according to General Procedure A, and purified by RP-MPLC (43 g C18 silica, gradient: 0% B over 3 CV, then 0→20% over 12 CV) to provide DKFZ-748 as white fluffy powder (98.8 mg, 0.300 mmol, 36% yield over two steps).

TLC R_{f} 0.28 (20% MeOH in CH₂Cl₂)

¹H NMR (400 MHz, DMSO-d₆) δ 10.37 (br s, 1H), 8.81 (br s, 1H), 8.43 (t, J=5.7 Hz, 1H), 8.27-8.20 (m, 1H), 8.05-7.92 (m, 2H), 7.62-7.49 (m, 4H), 3.42 (app q, J=6.5 Hz, 2H), 2.54 (t, J=6.8 Hz, 2H), 2.35 (t, J=7.2 Hz, 2H), 2.23 (s, 3H), 1.99 (t, J=7.4 Hz, 2H), 1.67 (app p, J=7.3 Hz, 2H) ppm.

¹³C NMR (101 MHz, DMSO-d₆) δ 169.1, 168.5, 135.2, 133.1, 129.7, 129.6, 128.2, 126.6, 126.2, 125.5, 125.0, 125.0, 56.6, 56.3, 41.9, 37.2, 30.2, 23.1 ppm.

HR-MS (m/z): [M+H]⁺ calcd for C₁₈H₂₄N₃O₃⁺: 330.1812; found: 330.1814.

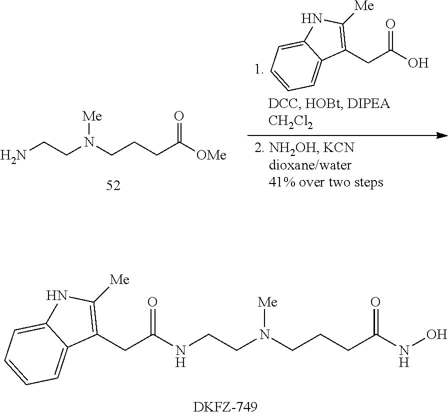

N-Hydroxy-4-(methyl(2-(2-(2-methyl-1H-indol-3-yl)acetamido)ethyl)amino)butanamide (DKFZ-749): The title compound was prepared from ester 52 2.HCl (84.6 mg, 0.342 mmol, 1.0 equiv) according to General Procedure B, but with DCC as coupling reagent. Precipitating DCU was removed by filtration and the concentrated filtrate was directly converted to the hydroxamic acid according to General Procedure A, and purified by HPLC Basic Method (gradient: 1→50% B in 12 min) to provide DKFZ-749 as an off-white solid (48.1 mg, 0.139 mmol, 41% yield over two steps).

TLC R_{f} 0.33 (20% MeOH in CH₂Cl₂)

¹H NMR (400 MHz, DMSO-d₆) δ 10.78 (br s, 1H), 9.45 (br s, 2H), 7.60 (t, J=5.6 Hz, 1H), 7.43 (dt, J=7.6, 1.0 Hz, 1H), 7.22 (dt, J=8.0, 1.0 Hz, 1H), 6.96 (ddd, J=8.0, 7.0, 1.3 Hz, 1H), 6.90 (ddd, J=8.0, 7.0, 1.2 Hz, 1H), 3.43 (s, 2H), 3.09 (app q, J=6.4 Hz, 2H), 2.33 (s, 3H), 2.29 (t, J=6.8 Hz, 2H), 2.22 (t, J=7.2 Hz, 2H), 2.08 (s, 3H), 1.91 (t, J=7.4 Hz, 2H), 1.55 (app p, J=7.3 Hz, 2H) ppm.

¹³C NMR (101 MHz, DMSO-d₆) δ 170.6, 169.0, 135.1, 133.0, 128.4, 119.9, 118.1, 117.8, 110.2, 104.9, 56.5, 56.3, 41.7, 36.7, 31.6, 30.2, 22.9, 11.4 ppm.

HR-MS (m/z): [M+H]⁺ calcd for C₁₈H₂₇N₄O₃⁺: 347.2078; found: 347.2077.

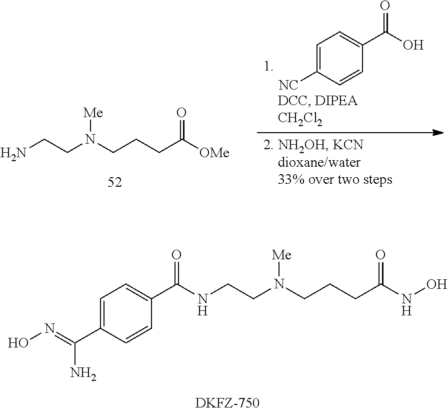

N-(2-((4-(Hydroxyamino)-4-oxobutyl)(methyl)amino)ethyl)-4-(M-hydroxycarbamimidoyl)benzamide (DKFZ-750): The title compound was prepared from ester 52 2.HCl (95.8 mg, 0.388 mmol, 1.0 equiv) according to General Procedure B, but with DCC as coupling reagent. Precipitating DCU was removed by filtration and the concentrated filtrate was directly converted to the hydroxamic acid according to General Procedure A, and purified by HPLC Basic Method (gradient: 1→20% B in 12 min) to provide DKFZ-750 as an off-white solid (42.6 mg, 0.126 mmol, 33% yield over two steps).

TLC R_{f} 0.27 (20% MeOH in CH₂Cl₂)

¹H NMR (400 MHz, DMSO-d₆) δ 9.58 (br s, 3H, —NH—OH and ═NOH), 8.48 (t, J=5.6 Hz, 1H, CONH), 7.86-7.79 (m, 2H, Ar H), 7.77-7.71 (m, 2H, Ar H), 5.88 (s, 2H, NH₂), 3.34 (app q, J=6.4 Hz, 2H, NHCH₂), 2.46 (t, J=6.9 Hz, 2H, NCH₂), 2.30 (t, J=7.1 Hz, 2H, NCH₂), 2.18 (s, 3H, Me), 1.95 (t, J=7.4 Hz, 2H, COCH₂), 1.61 (app p, J=7.3 Hz, 2H, CH₂—CH₂—CH₂) ppm.

¹³C NMR (101 MHz, DMSO-d₆) δ 168.9 (CONHOH), 165.7 (CONH), 150.2 (HON═CNH₂), 135.7 (Ar C), 134.7 (Ar C), 127.0 (2Ar CH), 125.1 (2Ar CH), 56.4 (CH₂), 56.1 (CH₂), 42.0 (Me), 37.3 (CH₂), 30.2 (CH₂), 23.0 (CH₂) ppm.

HR-MS (m/z): [M+Na]⁺ calcd for C₁₅H₂₃N₅NaO₄⁺: 360.1642; found: 360.1646.

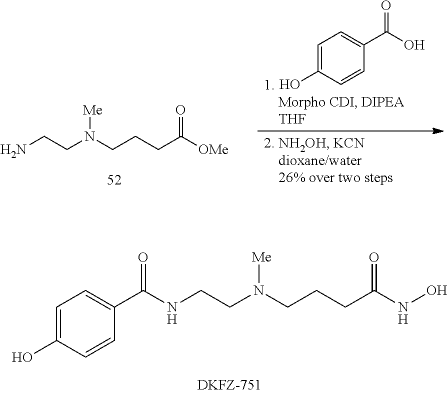

4-Hydroxy-N-(2-((4-(hydroxyamino)-4-oxobutyl)(methyl)amino)ethyl)benzamide (DKFZ-751): The title compound was prepared from ester 52 2.HCl (103.8 mg, 0.420 mmol, 1.0 equiv) according to General Procedure B, but in THF with Morpho CDI as coupling reagent. The crude product was directly converted to the hydroxamic acid according to General Procedure A, and purified by HPLC Acidic Method but with 0.05% formic acid as pH modifier (gradient: 1→8% B in 12 min) to provide the formate salt of DKFZ-751 as an orange-red amorphous solid (37.1 mg, 0.109 mmol, 26% yield over two steps).

¹H NMR (400 MHz, D₂O) δ 8.42 (s, 1H), 7.66 (d, J=7.5 Hz, 2H), 6.91 (d, J=7.5 Hz, 2H), 3.72 (t, J=5.6 Hz, 2H), 3.37 (t, J=5.6 Hz, 2H), 3.29-3.14 (m, 2H), 2.91 (s, 3H), 2.26 (t, J=6.9 Hz, 2H), 2.01 (app p, J=7.5 Hz, 2H) ppm.

¹³C NMR (101 MHz, D₂O) δ 173.9, 173.8 (br, HCO₂⁻), 173.6, 162.5, 132.3, 127.2, 118.3, 58.2, 58.1, 43.1, 37.7, 31.9, 22.4 ppm.

HR-MS (m/z): [M+H]⁺ calcd for C₁₄H₂₂N₃O₄⁺: 296.1605; found: 296.1607.

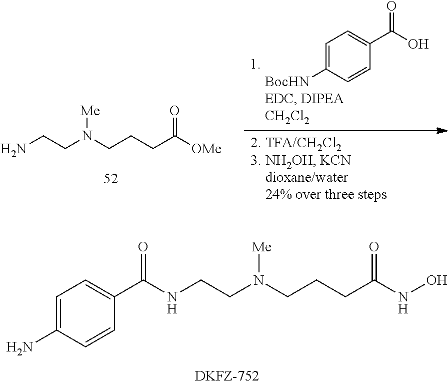 [SMILES: "1.    EDC, DIPEA    CH2Cl2" ; CC(C)(C)OC(=O)Nc1ccc(C(=O)O)cc1 ; COC(=O)CCCN(C)CCN ; "2. TFA/CH2Cl23. NH2OH, KCN    dioxane/water    24% over three steps" ; CN(CCCC(=O)NO)CCNC(=O)c1ccc(N)cc1]

4-Amino-N-(2-((4-(hydroxyamino)-4-oxobutyl)(methyl)amino)ethyl)benzamide (DKFZ-752): The title compound was prepared from ester 52 2.HCl (103.8 mg, 0.420 mmol, 1.0 equiv) according to General Procedure B. The crude product was dissolved in TFA/CH₂Cl₂ (5 mL, 20% TFA in CH₂Cl₂), stirred at rt for 1 h, then concentrated and residual TFA coevaporated with MeOH. The crude TFA salt was directly converted to the hydroxamic acid according to General Procedure A, and purified by HPLC Basic Method (gradient: 1→30% B in 12 min) to provide DKFZ-752 as off-white solid (30.2 mg, 0.103 mmol, 24% yield over three steps).

¹H NMR (400 MHz, DMSO-d₆) δ 8.42 (s, 1H), 10.66-8.26 (m, 2H), 7.87 (t, J=5.6 Hz, 1H), 7.58-7.49 (m, 2H), 6.56-6.48 (m, 2H), 5.57 (s, 2H), 3.27 (app q, J=6.4 Hz, 2H), 2.41 (t, J=7.1 Hz, 2H), 2.29 (t, J=7.2 Hz, 2H), 2.16 (s, 3H), 1.95 (t, J=7.4 Hz, 2H), 1.61 (app p, J=7.2 Hz, 2H) ppm.

¹³C NMR (101 MHz, DMSO-d₆) δ 169.1, 166.1, 151.5, 128.6, 121.3, 112.5, 56.5, 56.4, 42.0, 37.0, 30.1, 22.9 ppm.

HR-MS (m/z): [M+H]⁺ calcd for C₁₄H₂₃N₄O₃⁺: 295.1765; found: 295.1768.

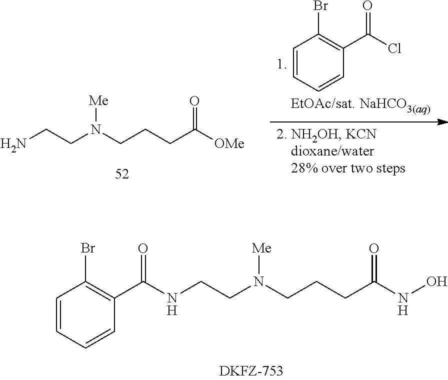

2-Bromo-N-(2-((4-(hydroxyamino)-4-oxobutyl)(methyl)amino)ethyl)benzamide (DKFZ-753): To 52 2.HCl (103.8 mg, 0.420 mmol, 1.0 equiv) dissolved in sat. NaHCO₃ solution (25 mL) and EtOAc (25 mL) in a separatory funnel was added 2-bromo benzoyl chloride (120 μL, 0.924 mmol, 2.2 equiv) in two portions, followed by vigorous shaking. Phases were separated and the organic layer was washed with dilute K₂CO₃ solution (2×25 mL) and brine (25 mL), then dried (MgSO₄) and concentrated. The residue was directly converted to the hydroxamic acid according to General Procedure A, and purified by HPLC Basic Method (gradient: 1→30% B in 15 min) to provide DKFZ-753 as an off-white solid (41.5 mg, 0.116 mmol, 28% yield over two steps).

¹H NMR (400 MHz, DMSO-d₆) δ 10.64-8.57 (br, 2H) 8.31 (t, J=5.7 Hz, 1H), 7.68-7.60 (m, 1H), 7.45-7.39 (m, 1H), 7.39-7.30 (m, 2H), 3.32-3.24 (m, overlapped with water peak), 2.46 (t, J=7.0 Hz, 2H), 2.30 (t, J=7.2 Hz, 2H), 2.18 (s, 3H), 1.97 (t, J=7.4 Hz, 2H), 1.62 (app p, J=7.3 Hz, 2H) ppm.

¹³C NMR (101 MHz, DMSO-d₆) δ 169.1, 167.1, 139.3, 132.7, 130.7, 128.7, 127.5, 118.9, 56.5, 56.0, 41.9, 37.2, 30.1, 23.0 ppm.

HR-MS (m/z): [M+H]⁺ calcd for C₁₄H₂₁BrN₃O₃⁺: 358.0761; found: 358.0764.

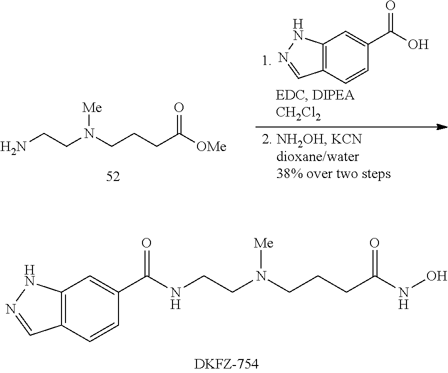

N-(2-((4-(Hydroxyamino)-4-oxobutyl)(methyl)amino)ethyl)-1H-indazole-6-carboxamide (DKFZ-754): The title compound was prepared from ester 52 2.HCl (65.1 mg, 0.263 mmol, 1.0 equiv) according to General Procedure B. The crude product was directly converted to the hydroxamic acid according to General Procedure A, and purified by HPLC Basic Method (gradient: 1→25% B in 12 min) to provide DKFZ-754 as a pale yellow solid (32.3 mg, 0.101 mmol, 38% yield over two steps).

¹H NMR (400 MHz, DMSO-d₆) δ 12.54-9.09 (br, 3H), 8.50 (t, J=5.7 Hz, 1H), 8.13 (d, J=0.9 Hz, 1H), 8.04 (s, 1H), 7.80 (dd, J=8.5, 0.9 Hz, 1H), 7.57 (dd, J=8.5, 1.4 Hz, 1H), 3.38 (app q, J=6.5 Hz, 2H), 2.56-2.45 (m, overlapped with DMSO signal), 2.33 (t, J=7.3 Hz, 2H), 2.20 (s, 3H), 1.97 (t, J=7.3 Hz, 2H), 1.64 (app p, J=7.3 Hz, 2H) ppm.

¹³C NMR (101 MHz, DMSO-d₆) δ 169.1, 166.5, 139.5, 133.4, 132.4, 124.2, 120.2, 119.2, 109.6, 56.5, 56.2, 42.0, 37.4, 30.2, 22.9 ppm.

HR-MS (m/z): [M+Na]⁺ calcd for C₁₅H₂₁N₅NaO₃⁺: 342.1537; found: 342.1541.

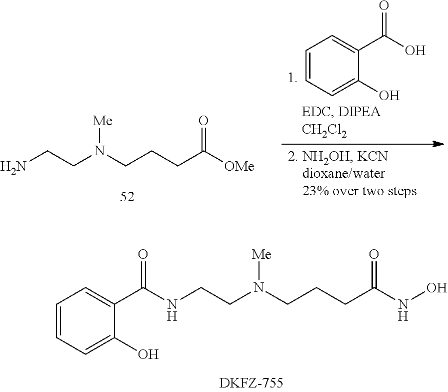

2-Hydroxy-N-(2-((4-(hydroxyamino)-4-oxobutyl)(methyl)amino)ethyl)benzamide (DKFZ-755): The title compound was prepared from ester 52 2.HCl (88.7 mg, 0.359 mmol, 1.0 equiv) according to General Procedure B. The crude product was directly converted to the hydroxamic acid according to General Procedure A, and purified by HPLC Basic Method (gradient: 1→5% B in 9 min) to provide DKFZ-755 as an off-white solid (24.9 mg, 0.084 mmol, 23% yield over two steps).

¹H NMR (400 MHz, DMSO-d₆) δ 10.34 (s, 1H), 8.97-8.42 (m, 1H), 8.77 (t, J=5.5 Hz, 1H), 7.82 (dd, J=7.9, 1.7 Hz, 1H), 7.43-7.34 (m, 1H), 6.93-6.83 (m, 2H), 3.42-3.36 (m, overlapped with water peak), 2.57-2.51 (m, 2H), 2.36 (t, J=7.3 Hz, 2H), 2.23 (s, 3H), 1.96 (t, J=7.4 Hz, 2H), 1.70-1.58 (m, 2H) ppm. The four Ar H show rotamer signals with a ratio of 1:0.16. Only the major rotamer is reported.

¹³C NMR (151 MHz, DMSO-d₆) δ 169.1, 168.5, 159.7, 133.5, 127.9, 118.6, 117.3, 115.8, 56.4, 55.6, 41.7, 36.8, 30.0, 22.7 ppm. Only signals of the major rotamer are reported.

HR-MS (m/z): [M+H]⁺ calcd for C₁₄H₂₂N₃O₄⁺: 296.1605; found: 296.1609.

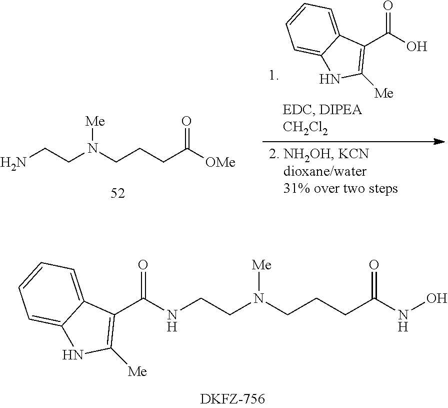

N-(2-((4-(Hydroxyamino)-4-oxobutyl)(methyl)amino)ethyl)-2-methyl-1H-indole-3-carboxamide (DKFZ-756): The title compound was prepared from ester 52 2.HCl (70.4 mg, 0.285 mmol, 1.0 equiv) according to General Procedure B. The crude product was directly converted to the hydroxamic acid according to General Procedure A, and purified by RP-MPLC (15.5 g C18 #69-2203-334, gradient: 0% B over 3 CV, then 0→15% over 12 CV) to provide DKFZ-756 as white, fluffy solid (29.7 mg, 0.089 mmol, 31% yield over two steps).

¹H NMR (400 MHz, DMSO-d₆) δ 11.44 (s, 1H), 10.80-8.18 (br, 2H), 7.78-7.71 (m, 1H), 7.35-7.28 (m, 1H), 7.20 (t, J=5.5 Hz, 1H), 7.11-7.01 (m, 2H), 3.41-3.33 (m, overlapped with water peak), 2.57 (s, 3H), 2.54-2.46 (m, overlapped with DMSO signal), 2.34 (t, J=7.2 Hz, 2H), 2.20 (s, 3H), 1.98 (t, J=7.5 Hz, 2H), 1.66 (app p, J=7.3 Hz, 2H) ppm.

¹³C NMR (101 MHz, DMSO-d₆) δ 169.0, 165.2, 139.3, 134.6, 125.9, 120.9, 119.9, 119.2, 110.9, 107.7, 56.7, 56.5, 41.7, 36.6, 30.2, 23.1, 13.2 ppm.

HR-MS (m/z): [M+H]⁺ calcd for C₁₇H₂₅N₄O₃⁺: 333.1921; found: 333.1921.

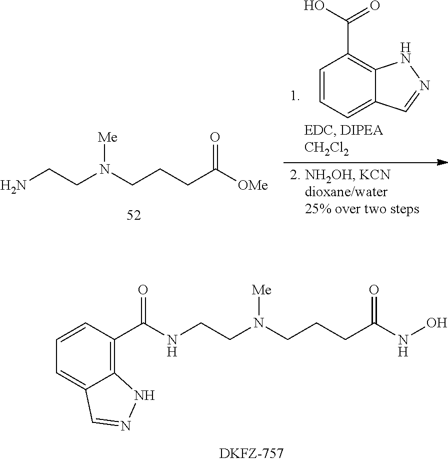

N-(2-((4-(Hydroxyamino)-4-oxobutyl)(methyl)amino)ethyl)-1H-indazole-7-carboxamide (DKFZ-757): The title compound was prepared from ester 52 2.HCl (196.0 mg, 0.793 mmol, 1.0 equiv) according to General Procedure B. The crude product was directly converted to the hydroxamic acid according to General Procedure A, and purified by HPLC Basic Method (gradient: 1→40% B in 12 min) to provide DKFZ-757 as white solid (67.0 mg, 0.202 mmol, 25% yield over two steps).

¹H NMR (400 MHz, DMSO-d₆) δ 8.73 (br s, 1H), 8.15 (s, 1H), 7.96-7.90 (m, 1H), 7.90-7.81 (m, 1H), 7.20-7.12 (m, 1H), 3.44 (app q, J=6.5 Hz, 2H), 2.52 (t, J=7.3 Hz, 2H), 2.34 (t, J=7.1 Hz, 2H), 2.21 (s, 3H), 2.00 (t, J=7.4 Hz, 2H), 1.65 (app p, J=7.4 Hz, 2H) ppm.

¹³C NMR (101 MHz, DMSO-d₆) δ 169.2, 165.7, 138.5, 133.0, 124.5, 124.2, 124.1, 119.4, 117.5, 56.6, 56.2, 41.9, 37.1, 30.1, 22.9 ppm.

HR-MS (m/z): [M+H]⁺ calcd for C₁₅H₂₂N₅O₃⁺: 320.1717; found: 320.1719.

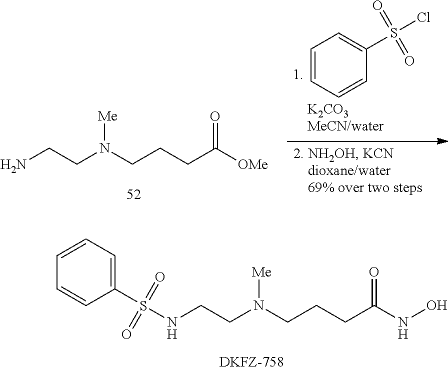

N-Hydroxy-4-(methyl(2-(phenylsulfonamido)ethyl)amino)butanamide (DKFZ-758): To a stirred solution of 52 2.HCl (121.9 mg, 0.493 mmol, 1.0 equiv) and K₂CO₃ (239 mg, 1.726 mmol, 3.5 equiv) in 12 mL MeCN/water (10:2) was added benzenesulfonyl chloride (82 μL, 0.641 mmol, 1.3 equiv) and stirred at rt until TLC indicated complete conversion, then concentrated in vacuo. The residue was dissolved in EtOAc (25 mL) and dilute K₂CO₃ solution (30 mL), phases were separated and the aqueous phase extracted with EtOAc (2×25 mL). Combined organic layers were washed with dilute K₂CO₃ solution (2×25 mL) and brine (25 mL), then dried (MgSO₄) and concentrated. The crude product was directly converted to the hydroxamic acid according to General Procedure A, and purified by HPLC Basic Method (gradient: 1→40% B in 13 min) to provide DKFZ-758 as white solid (112.3 mg, 0.341 mmol, 69% yield over two steps).

TLC R_{f} 0.33 (20% MeOH in CH₂Cl₂)

¹H NMR (400 MHz, DMSO-d₆) δ 8.83 (br s, 2H), 7.81 (d, J=7.2 Hz, 2H), 7.71-7.52 (m, 3H), 2.81 (t, J=7.0 Hz, 2H), 2.28 (t, J=7.0 Hz, 2H), 2.17 (t, J=7.2 Hz, 2H), 2.02 (s, 3H), 1.90 (t, J=7.4 Hz, 2H), 1.52 (app p, J=7.3 Hz, 2H) ppm.

¹³C NMR (101 MHz, DMSO-d₆) δ 169.0, 140.6, 132.3, 129.2, 126.5, 56.4, 56.2, 41.6, 40.6, 30.1, 22.8 ppm.

HR-MS (m/z): [M+H]⁺ calcd for C₁₃H₂₂N₃O₄S⁺: 316.1326; found: 316.1328.

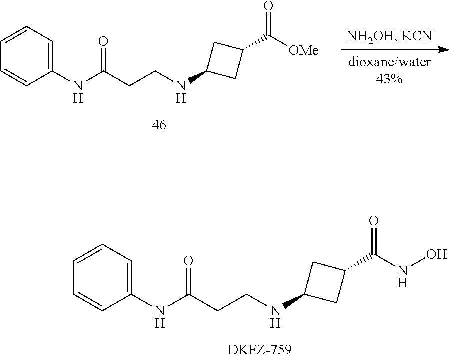

(1r,3r)-N-Hydroxy-3-((3-oxo-3-(phenylamino)propyl)amino)cyclobutanecarboxamide (DKFZ-759): 46 (131.2 mg, 0.475 mmol, 1.0 equiv) was converted to the hydroxamic acid according to General Procedure A, and purified by RP-MPLC (15.5 g C18 #69-2203-334, gradient: 0% B over 3 CV, then 0→20% over 20 CV). The product was triturated with EtOAc (twice) and Et₂O (once) to provide DKFZ-759 as a white solid (56.3 mg, 0.203 mmol, 43% yield).

¹H NMR (400 MHz, DMSO-d₆) δ 10.29 (br s, 1H), 10.02 (s, 1H), 8.66 (br s, 1H), 7.62-7.53 (m, 2H), 7.33-7.23 (m, 2H), 7.01 (tt, J=7.4, 1.2, 1.2 Hz, 1H), 3.43-3.24 (m, overlapped with water peak), 2.79-2.66 (m, 3H), 2.40 (t, J=6.7 Hz, 2H), 2.29-2.18 (m, 2H), 1.91-1.79 (m, 2H) ppm.

¹³C NMR (101 MHz, DMSO-d₆) δ 172.0, 170.5, 139.3, 128.7, 123.0, 119.1, 51.2, 42.6, 37.1, 32.5, 30.7

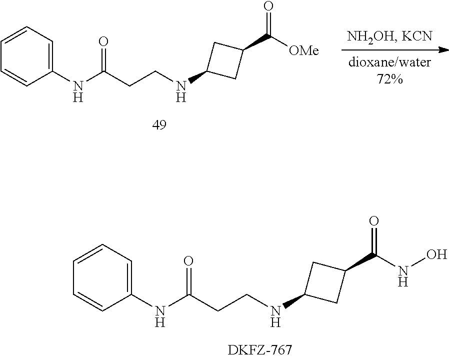

(1s,3s)-N-Hydroxy-3-((3-oxo-3-(phenylamino)propyl)amino)cyclobutanecarboxamide (DKFZ-767): 49 (138.6 mg, 0.502 mmol, 1.0 equiv) was converted to the hydroxamic acid according to General Procedure A, and purified by RP-MPLC (15.5 g C18Aq #69-2203-559, gradient: 0% B over 3 CV, 0→20% over 20 CV, then 20% over 10 CV) to provide DKFZ-767 as a white solid (100.2 mg, 0.361 mmol, 72% yield).

¹H NMR (600 MHz, DMSO-d₆) δ 10.34 (br s, 1H), 10.03 (s, 1H), 9.12-8.28 (br, 1H), 7.60-7.55 (m, 2H), 7.31-7.24 (m, 2H), 7.01 (tt, J=7.4, 1.2 Hz, 1H), 3.12-2.98 (m, 1H), 2.71 (t, J=6.8 Hz, 2H), 2.46-2.41 (m, 1H), 2.41-2.37 (m, 2H), 2.25-2.17 (m, 2H), 1.88-1.76 (m, 2H) ppm.

¹³C NMR (151 MHz, DMSO-d₆) δ 170.5, 170.5, 139.3, 128.7, 122.9, 119.0, 49.5, 42.5, 37.1, 33.6, 29.1 ppm.

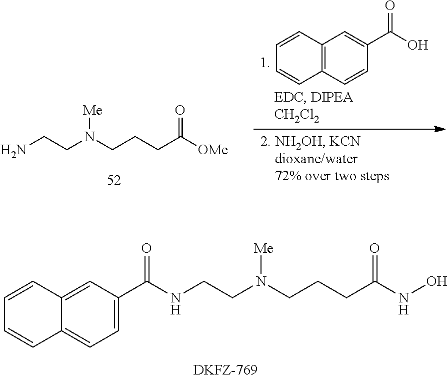

N-(2-((4-(Hydroxyamino)-4-oxobutyl)(methyl)amino)ethyl)-2-naphthamide (DKFZ-769): The title compound was prepared from ester 52 2.HCl (86.1 mg, 0.348 mmol, 1.0 equiv) according to General Procedure B. The crude product was directly converted to the hydroxamic acid according to General Procedure A, and purified by RP-MPLC (15.5 g C18Aq #69-2203-559, gradient: 0% B over 3 CV, then 0→20% over 22 CV, then 50% B for 4 CV) to provide DKFZ-769 as white solid (82.8 mg, 0.251 mmol, 72% yield over two steps).

¹H NMR (600 MHz, DMSO-d₆) δ 9.53 (br s, 2H), 8.58 (t, J=5.7 Hz, 1H), 8.44 (s, 1H), 8.05-7.89 (m, 4H), 7.64-7.56 (m, 2H), 3.41 (app q, J=6.6 Hz, 2H), 2.52 (t, J=7.1 Hz, 2H), 2.34 (t, J=7.2 Hz, 2H), 2.21 (s, 3H), 1.99 (t, J=7.4 Hz, 2H), 1.65 (app p, J=7.3 Hz, 2H) ppm.

¹³C NMR (151 MHz, DMSO-d₆) δ 169.1, 166.2, 134.1, 132.2, 132.0, 128.8, 127.8, 127.6, 127.5, 127.3, 126.7, 124.1, 56.5, 56.2, 42.0, 37.4, 30.2, 22.9 ppm.

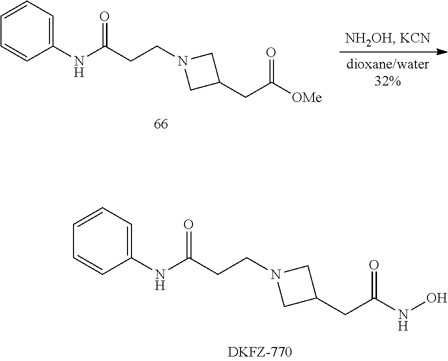

3-(3-(2-(Hydroxyamino)-2-oxoethyl)azetidin-1-yl)-N-phenylpropanamide (DKFZ-770): 66 (235 mg, 0.849 mmol, 1.0 equiv) was converted to the hydroxamic acid according to General Procedure A, and purified by RP-MPLC (15.5 g C18Aq #69-2203-559, gradient: 0% B over 3 CV, then 0→15% over 25 CV) to provide DKFZ-770 as a white solid (76.0 mg, 0.274 mmol, 32% yield).

¹H NMR (600 MHz, DMSO-d₆) δ 9.98 (s, 1H), 7.58 (d, J=8.1 Hz, 2H), 7.28 (t, J=7.8 Hz, 2H), 7.01 (t, J=7.4 Hz, 1H), 3.28 (t, J=7.0 Hz, 2H), 2.75 (t, J=6.6 Hz, 2H), 2.61 (t, J=7.0 Hz, 2H), 2.59-2.52 (m, 1H), 2.27 (t, J=6.9 Hz, 2H), 2.19 (d, J=7.7 Hz, 2H) ppm.

¹³C NMR (151 MHz, DMSO-d₆) δ 170.0, 167.7, 139.3, 128.7, 123.0, 119.0, 59.5, 55.0, 36.9, 35.1, 27.4 ppm.

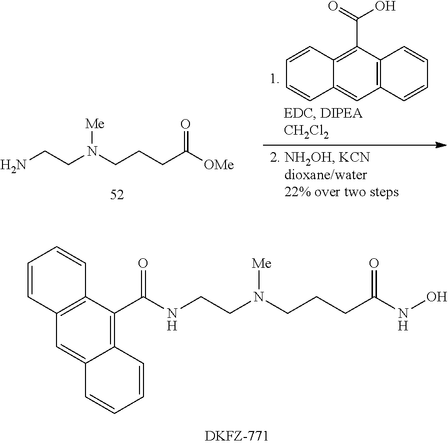

N-(2-((4-(Hydroxyamino)-4-oxobutyl)(methyl)amino)ethyl)anthracene-9-carboxamide (DKFZ-771): The title compound was prepared from ester 52 2.HCl (92.7 mg, 0.375 mmol, 1.0 equiv) according to General Procedure B. The crude product was purified by MPLC (12 g silica, gradient: 0→4% MeOH in CH₂Cl₂ over 13 CV, then 4% MeOH for 20 CV, then 4→8% MeOH over 10 CV) to provide the corresponding methyl ester of DKFZ-771 (62.3 mg, m/z: [M+H]⁺: 379.2), which was directly converted to DKFZ-771 according to General Procedure A, and purified by RP-MPLC (15.5 g C18Aq #69-2203-559, gradient: 0% B over 2 CV, 0→22% B over 12 CV, 22% B for 9 CV, 22→36% B over 5 CV, then 36% B over 5 CV) to provide DKFZ-771 as off-white fluffy powder (31.2 mg, 0.082 mmol, 22% yield over two steps).

¹H NMR (600 MHz, DMSO-d₆) δ 11.09-9.48 (br, 2H partly exchanged), 8.74 (t, J=5.8 Hz, 1H), 8.64 (s, 1H), 8.14-8.10 (m, 2H), 8.09-8.04 (m, 2H), 7.60-7.51 (m, 4H), 3.58 (app q, J=6.3 Hz, 2H), 2.62 (t, J=6.6 Hz, 2H), 2.40 (t, J=7.2 Hz, 2H), 2.29 (s, 3H), 2.01 (t, J=7.5 Hz, 2H), 1.73 (app p, J=7.5 Hz, 2H) ppm.

¹³C NMR (151 MHz, DMSO-d₆) δ 169.1, 168.1, 133.5, 130.7, 128.3, 127.3, 127.0, 126.3, 125.5 (2 Ar CH), 56.8, 56.6, 41.7, 37.2, 30.3, 23.2 ppm.

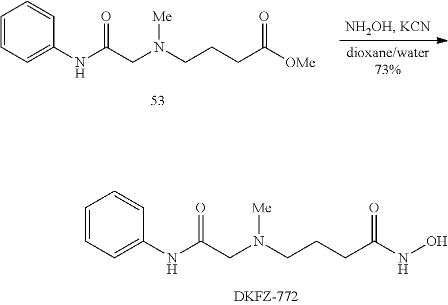

N-Hydroxy-4-(methyl(2-oxo-2-(phenylamino)ethyl)amino)butanamide (DKFZ-772): 53 (165 mg, 0.625 mmol, 1.0 equiv) was converted to the hydroxamic acid according to General Procedure A, and purified by RP-MPLC (15.5 g C18Aq #69-2203-559, gradient: 0→20% B over 25 CV, then 20% B over 10 CV) to provide DKFZ-772 as an off-white solid (121 mg, 0.457 mmol, 73% yield).

¹H NMR (600 MHz, DMSO-d₆) δ 10.11-8.98 (br, 3H), 7.69-7.63 (m, 2H), 7.32-7.27 (m, 2H), 7.05 (tt, J=7.4, 1.2 Hz, 1H), 3.09 (s, 2H), 2.41 (t, J=7.1 Hz, 2H), 2.25 (s, 3H), 2.01 (t, J=7.3 Hz, 2H), 1.69 (app p, J=7.0 Hz, 2H) ppm.

¹³C NMR (151 MHz, DMSO-d₆) δ 169.3, 169.0, 138.6, 128.6, 123.3, 119.4, 61.5, 56.7, 42.4, 30.4, 23.0 ppm.

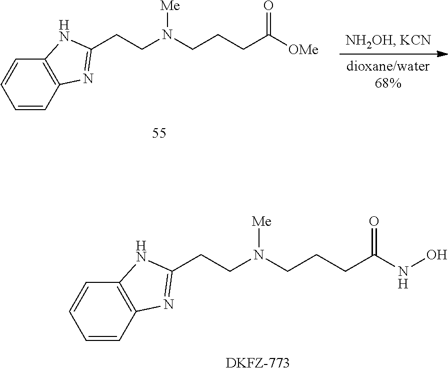

4-((2-(Benzimidazol-2-yl)ethyl)(methyl)amino)-N-hydroxybutanamide (DKFZ-773): The title compound was prepared from ester 55 (178 mg, 0.646 mmol, 1.0 equiv) according to General Procedure A, and purified by RP-MPLC (15.5 g C18Aq #69-2203-559, gradient: 0% B over 6 CV, 0→18% B over 25 CV, then 18% B over 4 CV) to provide DKFZ-773 as white solid (122 mg, 0.442 mmol, 68% yield).

¹H NMR (600 MHz, DMSO-d₆) δ 13.37-9.78 (br, 2H), 9.71-7.75 (br, 1H), 7.50-7.41 (m, 2H, Ar H), 7.13-7.07 (m, 2H, Ar H), 2.93 (t, J=7.5 Hz, 2H, NCH₂), 2.76 (t, J=7.5 Hz, 2H, Ar—CH₂), 2.33 (t, J=7.1 Hz, 2H, NCH₂), 2.18 (s, 3H, Me), 1.95 (t, J=7.4 Hz, 2H, COCH₂), 1.63 (app p, J=7.3 Hz, 2H, CH₂—CH₂—CH₂) ppm.

¹³C NMR (151 MHz, DMSO-d₆) δ 169.2 (CONHOH), 153.9 (N═CNH), 121.1 (Ar CH), 55.9 (CH₂), 55.3 (Ar—CH₂), 41.6 (Me), 30.1 (CH₂), 26.6 (CH₂), 22.8 (CH₂) ppm. Due to dynamics of the benzimidazole NH, some benzimidazole carbon signals are too broad to be identified in ¹³C spectra.

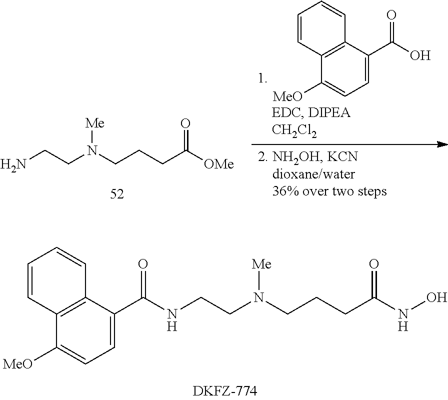

N-(2-((4-(Hydroxyamino)-4-oxobutyl)(methyl)amino)ethyl)-4-methoxy-1-naphthamide (DKFZ-774): The title compound was prepared from ester 52 2.HCl (113.7 mg, 0.460 mmol, 1.0 equiv) according to General Procedure B. The crude product was directly converted to the hydroxamic acid according to General Procedure A, and purified by RP-MPLC (15.5 g C18Aq #69-2203-559, gradient: 0→4% B over 12 CV, 4→20% B over 16 CV, then 20% B over 2 CV) to provide DKFZ-774 as white fluffy powder (59.7 mg, 0.166 mmol, 36% yield over two steps).

¹H NMR (600 MHz, DMSO-d₆) δ 10.26-8.91 (br, 2H), 8.32 (d, J=8.4 Hz, 1H), 8.29 (t, J=5.7 Hz, 1H), 8.20 (d, J=8.4 Hz, 1H), 7.59-7.55 (m, 2H), 7.55-7.50 (m, 1H), 6.98 (d, J=8.0 Hz, 1H), 4.00 (s, 3H), 3.39 (app q, J=6.5 Hz, 2H), 2.52 (t, J=6.9 Hz, 2H), 2.34 (t, J=7.2 Hz, 2H), 2.22 (s, 3H), 1.98 (t, J=7.4 Hz, 2H), 1.66 (app p, J=7.3 Hz, 2H) ppm.

¹³C NMR (151 MHz, DMSO-d₆) δ 169.1, 168.4, 155.9, 131.1, 127.1, 127.0, 126.3, 125.6, 125.5, 124.7, 121.6, 103.1, 56.6, 56.3, 55.8, 41.9, 37.2, 30.2, 23.1 ppm.

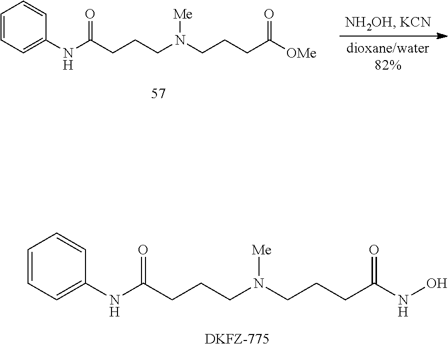

N-Hydroxy-4-(methyl(4-oxo-4-(phenylamino)butyl)amino)butanamide (DKFZ-775): 57 (156 mg, 0.535 mmol, 1.0 equiv) was converted to the hydroxamic acid according to General Procedure A, and purified by RP-MPLC (15.5 g C18Aq #69-2203-559, gradient: 0% B over 4 CV, 0→20% B over 23 CV, then 20% B over 10 CV) to provide DKFZ-775 as a white solid (129 mg, 0.440 mmol, 82% yield).

¹H NMR (600 MHz, DMSO-d₆) δ 11.13-8.04 (br, 2H), 9.87 (s, 1H), 7.62-7.56 (m, 2H), 7.30-7.23 (m, 2H), 7.01 (tt, J=7.3, 1.2 Hz, 1H), 2.36-2.26 (m, 4H), 2.24 (t, J=7.3 Hz, 2H), 2.11 (s, 3H), 1.96 (t, J=7.5 Hz, 2H), 1.70 (app p, J=7.3 Hz, 2H), 1.61 (app p, J=7.4 Hz, 2H) ppm.

¹³C NMR (151 MHz, DMSO-d₆) δ 171.2, 169.2, 139.4, 128.6, 122.9, 119.0, 56.5 (2CH₂ overlapped), 41.7, 34.3, 30.2, 22.9, 22.8 ppm.

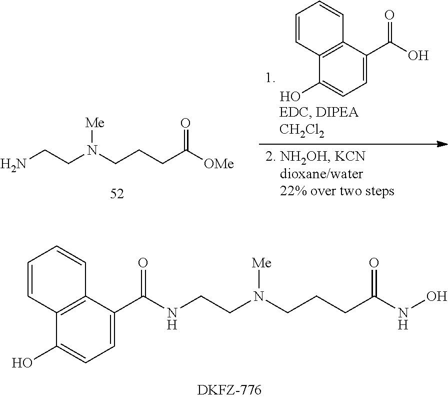

4-Hydroxy-N-(2-((4-(hydroxyamino)-4-oxobutyl)(methyl)amino)ethyl)-1-naphthamide (DKFZ-776): The title compound was prepared from ester 52 2.HCl (71.3 mg, 0.288 mmol, 1.0 equiv) and 4-hydroxy-1-naphthoic acid⁴⁹ according to General Procedure B. The crude product was purified by MPLC (4 g silica, gradient: 0→5% MeOH in CH₂Cl₂ over 20 CV, 5% MeOH for 30 CV, then 5→20% MeOH over 20 CV) to provide the corresponding methyl ester of DKFZ-776 (78.4 mg, m/z: [M+H]⁺: 345.2, [M−H]⁻: 343.2), which was directly converted to DKFZ-776 according to General Procedure A, and purified by RP-MPLC (15.5 g C18Aq #69-2203-559, gradient: 0% B over 6 CV, 0 →14% over 20 CV, 14→17% B over 10 CV, then 17% B over 4 CV) to provide DKFZ-776 as off-white solid (22.4 mg, 0.065 mmol, 22% yield over two steps).

¹H NMR (600 MHz, DMSO-d₆) δ 10.98-9.72 (br, 2H), 8.31 (d, J=8.5 Hz, 1H), 8.21-8.14 (m, 2H), 7.55-7.49 (m, 1H), 7.49-7.43 (m, 2H), 6.84 (d, J=7.8 Hz, 1H), 3.37 (app q, J=6.5 Hz, 2H), 2.54-2.48 (m, overlapped with DMSO signal), 2.34 (t, J=7.2 Hz, 2H), 2.21 (s, 3H), 1.99 (t, J=7.4 Hz, 2H), 1.66 (app p, J=7.4 Hz, 2H) ppm.

¹³C NMR (151 MHz, DMSO-d₆) δ 169.2, 168.6, 155.0, 131.6, 126.7, 126.7, 125.5, 125.2, 124.7, 124.5, 122.2, 106.6, 56.6, 56.3, 41.9, 37.2, 30.2, 23.0 ppm.

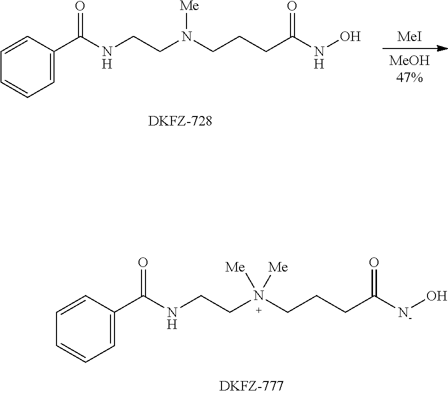

(4-((2-Benzamidoethyl)dimethylammonio)butanoyl)(hydroxy)amide (DKFZ-777): To a stirred solution of DKFZ-728 (40.0 mg, 0.143 mmol, 1.0 equiv) in MeOH (1 mL) was added Mel (90.7 μL, 1.457 mmol, 10.2 equiv) in small portions over 7 h and stirred at rt until LC/MS indicated complete conversion. The reaction mixture was concentrated in vacuo, dissolved in 0.1% aqueous ammonia (0.5 mL) and purified by cation exchange chromatography (500 mg (solute SCX-2 (Biotage), gradient: 2 CV MeOH, then 8 CV 0.2 M aqueous HBr). Aqueous fractions were diluted with water and lyophilized. The product was further purified by HPLC Basic Method (gradient: 1→25% B in 11 min, then 25% B for 7 min) to provide the internal salt of DKFZ-777 as off-white solid (19.8 mg, 0.068 mmol, 42% yield).

Note: HPLC purification was required due to the formation of the carboxylic acid after elution with 0.2 M aqueous HBr. DKFZ-777 and the corresponding carboxylic acid are poorly soluble in water.

¹H NMR (600 MHz, DMSO-d₆) δ 9.36 (br s, 1H), 7.91 (d, J=7.7 Hz, 2H), 7.54 (t, J=7.2 Hz, 1H), 7.48 (t, J=7.2 Hz, 2H), 3.67 (t, J=6.5 Hz, 2H), 3.47 (t, J=6.5 Hz, 2H), 3.43-3.30 (br s, CH₂ under water peak) 3.08 (s, 6H), 2.01-1.73 (m, 4H) ppm.

¹³C NMR (151 MHz, DMSO-d₆) δ 166.6, 165.6, 133.7, 131.5, 128.3, 127.3, 63.0, 61.3, 50.8, 33.3, 29.8, 18.9 ppm.

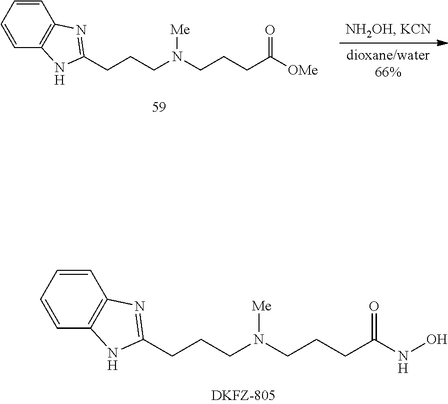

4-((3-(Benzimidazol-2-yl)propyl)(methyl)amino)-N-hydroxybutanamide (DKFZ-805): 59 (118 mg, 0.408 mmol, 1.0 equiv) was converted to the hydroxamic acid according to General Procedure A, and purified by RP-MPLC (15.5 g C18Aq #69-2203-559, gradient: 0% B over 8 CV, 0→19% B over 22 CV, then 19% B over 5 CV) to provide DKFZ-805 as a colorless solid (78.6 mg, 0.271 mmol, 66% yield).

¹H NMR (600 MHz, DMSO-d₆) δ 12.17 (br s, 1H), 10.47 (br s, 1H), 9.43-8.42 (br, 1H), 7.50-7.41 (m, 2H), 7.12-7.08 (m, 2H), 2.81 (t, J=7.7 Hz, 2H), 2.33 (t, J=7.0 Hz, 2H), 2.25 (t, J=7.1 Hz, 2H), 2.12 (s, 3H), 1.98 (t, J=7.4 Hz, 2H), 1.87 (app p, J=7.3 Hz, 2H), 1.62 (app p, J=7.3 Hz, 2H) ppm.

¹³C NMR (151 MHz, DMSO-d₆) δ 169.2, 155.2, 127.41-98.12 (m), 121.1, 56.6, 56.3, 41.7, 30.2, 26.4, 25.4, 22.9 ppm. Due to dynamics of the benzimidazole NH, some benzimidazole carbon signals are too broad to be identified in ¹³C spectra.

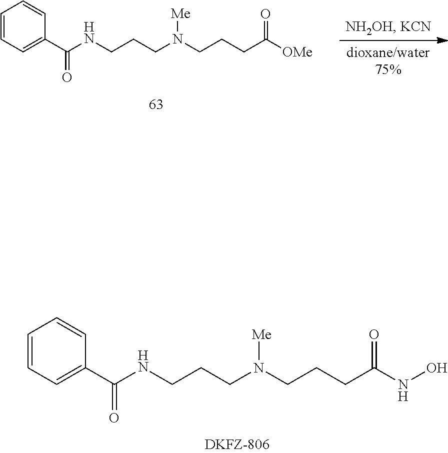

N-(3-((4-(Hydroxyamino)-4-oxobutyl)(methyl)amino)propyl)benzamide (DKFZ-806): 63 (162 mg, 0.552 mmol, 1.0 equiv) was converted to the hydroxamic acid according to General Procedure A, and purified by RP-MPLC (15.5 g C18Aq #69-2203-559, gradient: 0% B over 5 CV, 0→11% B over 15 CV, then 11% B over 20 CV) to provide DKFZ-806 as a white solid (120 mg, 0.412 mmol, 75% yield).

¹H NMR (400 MHz, DMSO-d₆) δ 10.34 (br s, 1H), 8.70 (br s, 1H), 8.48 (t, J=5.6 Hz, 1H), 7.91-7.76 (m, 2H), 7.54-7.41 (m, 3H), 3.27 (td, J=7.1, 5.6 Hz, 2H), 2.32 (t, J=7.1 Hz, 2H), 2.25 (t, J=7.2 Hz, 2H), 2.12 (s, 3H), 1.96 (t, J=7.3 Hz, 2H), 1.73-1.54 (m, 4H) ppm.

¹³C NMR (101 MHz, DMSO-d₆) δ 169.1, 166.1, 134.7, 131.0, 128.2, 127.1, 56.6, 55.0, 41.7, 37.8, 30.2, 26.8, 22.9 ppm.

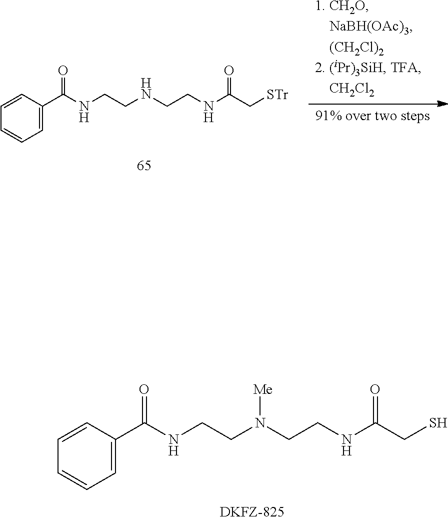

N-(2-((2-(2-Mercaptoacetamido)ethyl)(methyl)amino)ethyl)benzamide (DKFZ-825): To a stirring solution of 65 (342 mg, approx. 0.522 mmol, 1.0 equiv) in (CH₂C1)₂ (8 mL) was added formaldehyde (37-41% aqueous solution, 0.101 mL, 1.312 mmol, 2.5 equiv), then NaBH(OAc)₃ (0.305 mg, 1.437 mmol, 2.7 equiv) and after stirring at rt for 45 min, the reaction was quenched by addition of 1 M NaOH (50 mL) and extracted with CH₂Cl₂ (3×30 mL). The combined organic layers were dried (MgSO₄) and concentrated. The crude product (m/z: [M+H]⁺ 538.2, TLC R_{f} 0.47 10% MeOH and 0.5% NH₄OH in CH₂Cl₂) was dissolved in CH₂Cl₂ (12 mL), TFA (1.26 mL, 16.31 mmol, 31 equiv), then (ⁱPr)₃SiH (0.241 mL, 1.176 mmol, 2.25 equiv) were added and the mixture was stirred until colorless. Then, the reaction mixture was diluted with CH₂Cl₂ (50 mL), sat. NaHCO₃ solution (75 mL) was added and stirred until no more bubbling occurred, then extracted with CH₂Cl₂ (4×50 mL) and the combined organic layers were dried (MgSO₄) and concentrated. The crude product was purified by MPLC (4 g silica, gradient: 0% for 5 CV, 0→20% for 12 CV, then 20% MeOH and 0.5% NH₄OH in CH₂Cl₂ for 15 CV) to provide DKFZ-825 as a colorless amorphous solid (140 mg, 0.474 mmol, approx. 91% yield over two steps).

TLC R_{f} 0.14 (10% MeOH and 0.5% NH₄OH in CH₂Cl₂).

¹H NMR (400 MHz, CDCl₃) δ 7.83-7.76 (m, 2H), 7.53-7.46 (m, 1H), 7.46-7.38 (m, 2H), 7.30 (br s, 1H), 6.98 (br s, 1H), 3.59 (app q, J=5.6 Hz, 2H), 3.40 (app q, J=5.6 Hz, 2H), 3.10 (s, 2H), 2.74 (t, J=5.5 Hz, 2H), 2.67 (t, J=5.6 Hz, 2H), 2.38 (s, 3H), 1.79 (s, 1H) ppm.

¹³C NMR (101 MHz, CDCl₃) δ 171.1, 168.2, 133.3, 132.2, 128.8, 127.3, 57.4, 56.9, 41.8, 35.7, 35.4, 28.1 ppm.

Note: The material was found to form the disulfide when exposed to air, especially in solution.

Synthesis of DTBTA-Eu³⁺-labelled Streptactin: To a solution of ATBTA-Eu³⁺ (39 mM; 60 μL, 2.3 μmol) from TCI, cat #A2083 in aqueous NaOAc (100 mM, pH 4.9) was added a solution of cyanuric chloride (93 mM in acetone; 25 μL, 2.3 μmol), then vortexed and mixed on a tube roller mixer at rt for 30 min. The solution was added dropwise to acetone (1 mL), the resulting suspension was centrifuged (10 000 rpm, 3 min), the precipitate was washed (3×) by resuspending in acetone (1 mL each time), followed by centrifugation, then air dried at 37° C. for 1 h. The precipitate was redissolved in sodium carbonate buffer (100 mM, pH 9.3; 400 μL). To 100 μL of this solution was added a solution of Strep-Tactin XT (5.0 mg, gift from IBA Life Sciences) in sodium carbonate buffer (100 mM, pH 9.3; 400 μL), then vortexed and mixed with a rotary mixer at 4° C. overnight. The reaction mixture was purified with a PD10 desalting column (17-0851-01, GE Healthcare), pre-saturated with BSA, using TBS buffer (50 mM Tris/HCl pH 7.5, 150 mM NaCl, 0.01% NaN₃). Concentration and labelling ratio of the product fraction was determined with a NanoDrop (ThernoFisher) spectrophotometer.

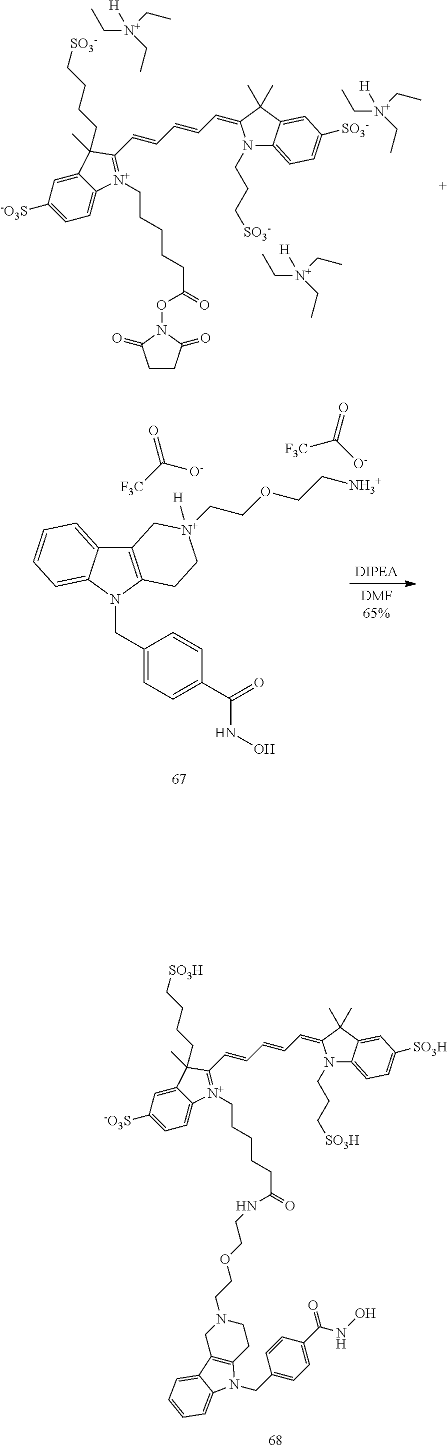

Tubastatin-AF647-Tracer (68): AF647 NHS ester (5.0 mg, 3.93 μmol, 1 equiv.) from Fluoroprobes, cat #1121-1, lot #10022 was mixed with 67⁴¹ (10.0 mg, 16 μmol, 4 equiv.) in 1.5 mL of DMF. To this solution was added DIPEA (5 μL, 27.0 μmol, 7 equiv). After stirring at rt for 30 min the solution was concentrated and then purified by HPLC under basic conditions with a gradient of 1-40% acetonitrile over 18 min to afford 68 (3.5 mg, 2.54 μmol, 65%) as a dark blue solid.

LC-MS m/z: [(M−2H)/2]²⁻ 630.2.

HDAC-Glo Assay for HDAC 1, 2, 3, 6 and 8: HDAC6 and class I inhibition was tested using the HDAC-GIo™ I/II Assay and Screening System (G6421, Promega) with recombinant human HDACs (BPS Bioscience; HDAC1 cat. #50051; HDAC2 cat. #50002; HDAC3/NcoR2 complex cat. #50003; HDAC6 cat. #50006; HDAC8 cat. #50008). The assay was carried out in a 384-well plate (4512, Corning) format according to the manufacturer's description. Inhibitors were tested at eight serial dilutions in triplicates ranging from 50 μM-86.7 μM (HDAC6) or 100 μM-8.67 nM (HDAC1,2,3,8). Drug dosing was performed from 10 mM and 0.1 mM DMSO stock solutions with a D300e Digital Dispenser (Tecan). HDACs (7 ng/mL for HDAC1, 10 ng/mL for HDAC2, 200 ng/mL for HDAC3/Ncor2 complex, 100 ng/mL for HDAC6, 200 ng/mL for HDAC8) and inhibitors were incubated together for 30 min at rt. After addition of the HDAC-Glo™ I/II reagent, plates were shaken (800 rpm orbital shaker, 30 s), centrifuged (300 g, 1 min) and incubated at rt for 30 min. Luminescence was detected with a CLARIOstar (BMG Labtech) plate reader. Luminescence signal was normalized with 100 μM SAHA treated negative controls and uninhibited positive controls. pIC₅₀-values were calculated from normalized BRET ratios using nonlinear regression log(inhibitor) four parameters least squares fit in GraphPad Prism version 7.04 for Windows, GraphPad Software, La Jolla Calif. USA, www.graphpad.com.

HDAC1/6 (ZMAL): Commercial available human recombinant HDAC1 (BPS Bioscience, catalog no. 50051) and human recombinant HDAC6 (BPS Bioscience, catalog no. 50006) were used. Activity assays were performed in OptiPlate™-96 F black microplates (PerkinElmer). Total assay volume of 60 μL contains 52 μL of enzyme solution in incubation buffer (50 mM Tris-HCl, pH 8.0, 137 mM NaCl, 2.7 mM KCl, 1 mM MgCl₂, and 1 mg/mL bovine serum albumin), 3 μL of increasing concentrations of inhibitors in DMSO and 5 μL of the fluorogenic substrate ZMAL (Z-(Ac)Lys-AMC) (126 μM). After incubation step (90 min, 37° C.) 60 μL of stop solution, containing 5 μL Trichostatin A (TSA) (33 μM) and 10 μL trypsin (6 mg/mL) in trypsin buffer (Tris-HCl 50 mM, pH 8.0, NaCl 100 mM), were added and the plate was incubated for 30 min at 37° C. Fluorescence signal was measured on a BMG LABTECH POLARstar OPTIMA plate reader (BMG Labtechnologies, Germany) with an excitation wavelength of 390 nm and an emission wavelength of 460 nm. Inhibition was measured at increasing concentration and IC₅₀ was calculated by nonlinear regression with Origin 9.0G software.

HDAC8 (FDL): For HDAC8 activity testing commercial available Fluor de Lys (FDL) drug discovery kit (BML-K1178) was used. Assay was performed according to the manufacturer's instructions. Enzyme solution (15 μL, obtained from C. Romier⁴⁶), increasing inhibitor concentrations (10 μL) and FDL substrate solution (25 μL) were incubated for 90 min at 37° C. in 1/2 AreaPlate-96 F microplates (PerkinElmer). Developer solution (50 μL) was added and the assay was incubated for 45 min at 30° C. Fluorescence signal and IC₅₀ was determined as mentioned for HDAC1/6.

Expression and purification of TwinStrepII-GST-HDAC10 for HDAC10 TR-FRET assay: A synthetic gene encoding TwinStrepII-GST-HDAC10 (human) was ordered from GeneArt (Thermo Fischer Scientific) and subcloned into the pFastBacl vector. The resulting construct was used for transposition in E. coli DH10EMBacY cells. The isolated bacmid DNA was then utilized to generate the recombinant baculovirus. For protein expression, 10 mL of baculovirus was added to 1 L of Sf21 cells at a density of 1×10⁶ cells/mL. The infected Sf21 cells were grown for 72 h in Sf-900 III SFM medium (Thermo Fischer Scientific) at 27° C. Cells were harvested by centrifugation and resuspended in running buffer (100 mM Tris pH 8.0, 150 mM NaCl, 1 mM EDTA and 1 mM DTT) supplemented with 10 mM MgCl₂, benzonase and cOmplete protease inhibitors (Merck). The cells were lysed using a Dounce homogenizer and the resulting lysate was centrifuged for 30 min at 4° C. at 125000× g in an ultracentrifuge. The clarified lysate was then loaded onto a 5 mL Strep-Tactin Superflow high capacity column (IBA) pre-equilibrated in running buffer. After sample loading and washing, the TwinStrepII-GST-HDAC10 protein was eluted in running buffer supplemented with 5 mM desthiobiotin (IBA). The elution fractions containing TwinStrepII-GST-HDAC10 were pooled and concentrated before being injected onto a HiLoad 16/600 Superdex 200 pg size exclusion chromatography column (GE Healthcare) pre-equilibrated with 25 mM HEPES/NaCl pH 7.5, 150 mM NaCl, 0.5 mM EDTA, 1 mM DTT and 10% glycerol. Samples were eluted from the size exclusion chromatography column in the same buffer, flash-frozen in liquid N₂ and stored at −80° C.

HDAC10 TR-FRET assay: TR-FRET assays were performed in white 384-well plates (4512, Corning) using 50 mM HEPES pH 8.0, 150 mM NaCl, 10 mM MgCl₂, 1 mM EGTA and 0.01% Brij-35 as buffer. The concentrations of reagent in 15 μL final assay volume were 5 nM TwinStrep-GST-HDAC10 (preparation described above), 25 nM “Tubastatin-AF647-Tracer” (synthesis described above) and 0.1 nM DTBTA-Eu³⁺-labelled Streptactin (synthesis described above). Inhibitors were tested at eight serial dilutions in triplicates ranging from 50 μM-86.7 μM and dosed from 10 mM and 0.1 mM DMSO stock solutions with a D300e Digital Dispenser (Tecan). After drug dosing to the premixed assay reagents in buffer, plates were shaken (800 rpm orbital shaker, 30 s), centrifuged (300 g, 1 min) and incubated at rt in the dark for 60 min. TR-FRET was measured with a CLARIOstar (BMG Labtech) plate reader, equipped with TR-FRET filters. Sample wells were excited with 100 flashes and fluorescence emission detected at 665 nm and 620 nm. FRET ratios were calculated from 665 nm/620 nm ratio and normalized for each plate using 50 μM SAHA treated negative controls and uninhibited positive controls. pIC50-values were calculated as described in the HDAC-Glo assay.

Zebrafish HDAC10 Assay (zHDAC10): All stock solutions were prepared in DMSO; Quisinostat (1 mM), NDA (16 mM) and Ac-spermidine-AMC (10 mM). Compounds for testing were solved and diluted to 12-fold higher than test concentration in DMSO. Ac-spermidine-AMC stock solutions was diluted with assay buffer (20 mM Na₂HPO₄, pH 7.9, 100 mM NaCl, 0.25 mM EDTA, 10% (v/v) glycerol, 10 mM Mesna, 0.01% TWEEN 20) to 126 μM. For assay determination stop solution was prepared, containing 5 μl NDA (16 mM), 5 μL Quisinostat (1 mM) and 190 μL borat buffer (100 mM boric acid, pH 9.5) per well. Directly before using enzyme solution (0.0054 mg/ml) was prepared in assay buffer.

The assay was performed in black 96-well plates (PerkinElmer, OptiPlate™-96 F). Assay buffer was presented in the plate, 55 μl for the blank, 45 μl for the blank containing enzyme solution, 50 μl for the negative control and 40 μl for the positive control and test compounds. 5 μl of DMSO were added to the wells of blanks, positive and negative control. Corresponding to the DMSO 5 μl of increasing concentrations of inhibitors in DMSO were added to the relevant wells. After adding 10 μL of zebrafish HDAC10 enzyme solution (0.045 mg/ml, obtained from D. Christianson⁶) to blank containing enzyme, positive control and test compounds, 5 μl Ac-spermidine-AMC solution (126 μM) were added to negative control, positive control and test compounds. The plate was incubated for 25 min at 25° C. Before measuring fluorescence (POLARstar plate reader, λₑₓ=330 nm, λₑₘ=390 nm) each well was filled with 200 μl stop solution.

Production of mono-clones stably expressing HDAC-nanoBRET proteins: Plasmids expressing a fusion of HDAC6 (containing only the 2nd catalytic domain) or HDAC10 with nanoluciferase were obtained from Promega (N2170). HeLa cells (0.75×10⁶) were seeded in a 6 cm dish and after 24 h were transfected with a mix of 10 μg plasmid and 3 μL Fugene in 200 μL OptiMEM. In detail, cells were washed with pre-warmed OptiMEM and subsequently overlaid with 2.3 mL of OptiMEM. After addition of 200 μL transfection mix, cells were incubated for 24 h at 37° C. Cells were than trypsinized and 0.2×10⁵ cells seeded into both 10 cm and 15 cm dishes. Transformants were selected with 1 mg/mL G-418 for 6 days with a media change after 3 days. Clones which formed colonies were picked by rinsing plates with 3 mL Trypsin/EDTA (Sigma T3924) followed by a 2 min incubation with 300 μL Trypsin/EDTA at 37° C. Colonies were than loosened and aspirated with a 10 μL filter tip and transferred to 24-well plates containing selection medium. Clones exhibiting a range of nanoluciferase activities were expanded and selected according to the highest BRET ratio.

Culture of stable BRET cell lines: Stably transfected HeLa cells were cultivated under sterile conditions in polystyrene cell culture flasks (658170, Greiner) at 37° C. and 5% CO₂ in a humidified atmosphere. D-MEM growth medium (D6049, Sigma) was supplemented with 10% FCS (FBS-12A, Capricorn Scientific), 1% Penicillin-Streptomycin (P4333, Sigma) and 1 mg/mL Geneticin (2039.3, Roth). At confluency, cells were passaged by removing old medium, DPBS (14190-094, gibco) wash, trypsination (T4049, Sigma) and seeding in fresh growth medium.

BRET Assay: The intracellular target engagement assay on HDAC6 and HDAC10 was performed as described by the kit manufacturer in a 96-well plate (3600, Corning) format with 1.9×10⁴ cells per well and a tracer concentration of 0.3 μM. Inhibitors were tested at ten 1:4 serial dilutions in triplicates ranging from 129 μM to 40 μM. Drug dosing was performed from 10 mM and 1 mM DMSO stock solutions with a D300e Digital Dispenser (Tecan), DMSO concentrations were normalized to 0.5% for all wells. Note: Due to the dosing increments of the drug printer, the dilution factor is not entirely stable over all dose levels. Assay plates were incubated at 37° C. for 2 h followed by measurement of 450 nm and 650 nm luminescence (80 nm bandwidth) at rt with a CLARIOstar (BMG Labtech) plate reader 2 min after NanoLuc substrate addition. BRET ratios were calculated from 650 nm/450 nm luminescence and normalized for each plate using 50 μM SAHA treated negative controls and uninhibited positive controls. pIC50-values were calculated as described in the HDAC Glo assay as described above.

TABLE 1

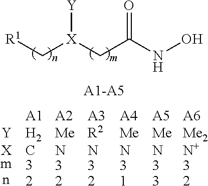

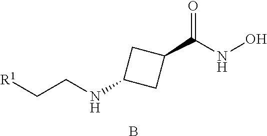

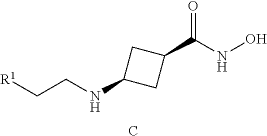

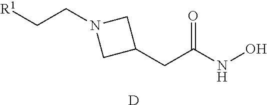

| | | | | | | | | | | | HDAC10/6 |
|---|---|---|---|---|---|---|---|---|---|---|---|
| | | | pIC₅₀ | | | | | | | | fold |
| Com- | R¹ | | HDAC1 | HDAC2 | HDAC3 | HDAC6 | HDAC8 | HDAC10 | HDAC6 | HDAC10 | selectivity |
| pound | (cap group) | Scaffold | Glo | Glo | Glo | Glo | Glo | FRET | BRET | BRET | (BRET) |
| SAHA | 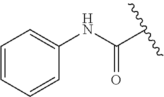 [SMILES: *C(=O)Nc1ccccc1] | A1 | 7.1 | 6.2 | 6.1 | 7.2 | 6.3 | 6.7 | 6.9 | 6.2 | 0.2 |
| DKFZ- 711 | 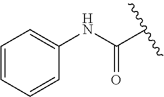 [SMILES: *C(=O)Nc1ccccc1] | A2 | 4.7 | −3.8 | 4.1 | 5.4 | 5.4 | 7.5 | 4.5 | 6.7 | 175 |
| DKFZ- 714 | 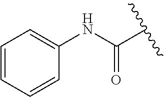 [SMILES: *C(=O)Nc1ccccc1] | A3 R² = Et | 4.3 | <3.7 | ~3.8 | 5.2 | 5.3 | 6.9 | −4.4 | 6.1 | ~200 |
| DKFZ- 715 | 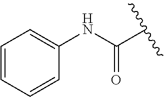 [SMILES: *C(=O)Nc1ccccc1] | A3 R² = i-Pr | 3.4 | <3.7 | >3.7 | 4.9 | 5.3 | 6.2 | <4.4 | 5.7 | >21 |
| DKFZ- 716 | 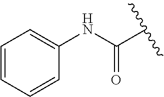 [SMILES: *C(=O)Nc1ccccc1] | A3 R² = n-Pr | 4.1 | <3.7 | ~3.7 | 5.3 | 5.1 | 6.5 | <4.4 | 6.0 | ~366 |
| DKFZ- 717 | 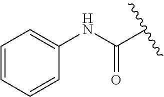 [SMILES: *C(=O)Nc1ccccc1] | A3 R² = n-Bu | 4.1 | <3.7 | ~3.7 | 5.3 | 5.1 | 6.2 | <4.4 | 5.8 | 38 |
| DKFZ- 718 | 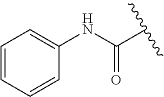 [SMILES: *C(=O)Nc1ccccc1] | A3 R² = benzyl | 4.6 | ~3.9 | ~4.0 | 6.0 | 5.6 | 5.7 | 5.0 | 6.3 | 25 |
| DKFZ- 724 | 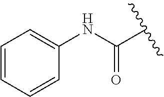 [SMILES: *C(=O)Nc1ccccc1] | A3 R² = cyclo- propyl | 4.4 | <3.7 | 3.9 | 5.5 | 5.7 | 6.6 | <4.4 | 6.4 | >110 |
| DKFZ- 728 | 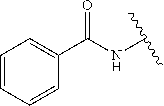 [SMILES: *NC(=O)c1ccccc1] | A2 | 4.5 | 4.3 | <4.0 | 5.1 | 4.8 | 7.9 | ~4.4 | 6.9 | 533 |
| DKFZ- 746 | 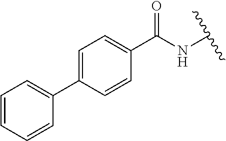 [SMILES: *NC(=O)c1ccc(-c2ccccc2)cc1] | A2 | 5.6 | ~4.4 | ~4.5 | 5.5 | 5.3 | 7.6 | NM | NM | |
| DKFZ- 747 | 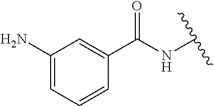 [SMILES: *NC(=O)c1cccc(N)c1] | A2 | 4.6 | <4.0 | <4.0 | 5.2 | 4.9 | 7.4 | NM | NM | |
| DKFZ- 748 | 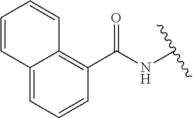 [SMILES: *NC(=O)c1cccc2ccccc12] | A2 | 4.9 | <4.0 | <4.0 | 5.6 | 5.9 | 8.2 | 4.8 | 7.7 | 662 |
| DKFZ- 749 | 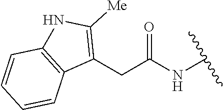 [SMILES: *NC(=O)Cc1c(C)[nH]c2ccccc12] | A2 | 5.0 | <4.0 | <4.0 | 5.5 | 5.6 | 7.1 | NM | NM | |
| DKFZ- 750 | 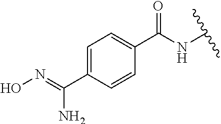 [SMILES: *NC(=O)c1ccc(/C(N)=N/O)cc1] | A2 | 4.8 | <4.0 | <4.0 | 5.2 | ~4.6 | 7.7 | <4.4 | 5.7 | >21 |
| DKFZ- 751 | 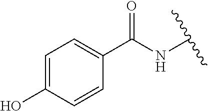 [SMILES: *NC(=O)c1ccc(O)cc1] | A2 | 4.8 | <4.0 | <4.0 | 5.7 | 4.6 | 8.1 | NM | NM | |
| DKFZ- 752 | 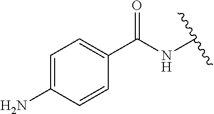 [SMILES: *NC(=O)c1ccc(N)cc1] | A2 | 4.8 | <4.0 | <4.0 | 5.4 | 4.7 | 7.8 | NM | NM | |
| DKFZ- 753 | 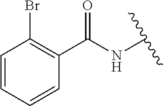 [SMILES: *NC(=O)c1ccccc1Br] | A2 | 4.6 | <4.0 | <4.0 | 5.2 | 5.1 | 7.8 | NM | NM | |
| DKFZ- 754 | 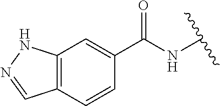 [SMILES: *NC(=O)c1ccc2cn[nH]c2c1] | A2 | 5.1 | <4.0 | <4.0 | 5.5 | 4.9 | 8.1 | NM | NM | |
| DKFZ- 755 | 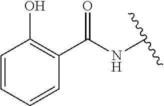 [SMILES: *NC(=O)c1ccccc1O] | A2 | 4.2 | <4.0 | <4.0 | 4.9 | 4.2 | 7.5 | NM | NM | |
| DKFZ- 756 | 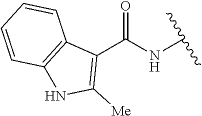 [SMILES: *NC(=O)c1c(C)[nH]c2ccccc12] | A2 | 5.1 | <4.0 | ~4.1 | 5.8 | 5.3 | 7.7 | NM | NM | |
| DKFZ- 757 | 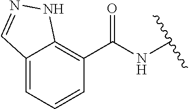 [SMILES: *NC(=O)c1cccc2cn[nH]c12] | A2 | 5.0 | <4.0 | <4.0 | 5.2 | ~4.6 | 8.1 | 4.6 | 7.2 | 423 |
| DKFZ- 758 | 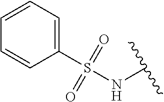 [SMILES: *NS(=O)(=O)c1ccccc1] | A2 | 4.6 | <4.0 | <4.0 | 5.2 | 5.2 | 7.5 | NM | NM | |
| DKFZ- 759 | 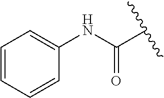 [SMILES: *C(=O)Nc1ccccc1] | C | 4.4 | <4.0 | <4.0 | 5.5 | 4.9 | 6.3 | NM | NM | |
| DKFZ- 767 | 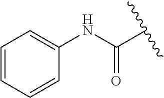 [SMILES: *C(=O)Nc1ccccc1] | D | 4.9 | 4.6 | ~4.0 | 5.7 | 5.1 | 6.9 | NM | NM | |
| DKFZ- 769 | 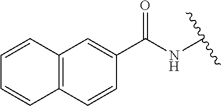 [SMILES: *NC(=O)c1ccc2ccccc2c1] | A2 | 5.6 | 5.3 | ~4.4 | 5.9 | 5.8 | 8.3 | NM | NM | |
| DKFZ- 770 | 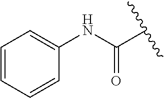 [SMILES: *C(=O)Nc1ccccc1] | B1 | 4.3 | 4.1 | <4.0 | 5.2 | 4.7 | 6.8 | <4.4 | 5.7 | >20 |
| DKFZ- 771 | 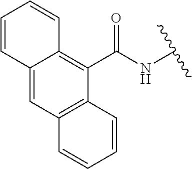 [SMILES: *NC(=O)c1c2ccccc2cc2ccccc12] | A2 | 5.7 | 5.4 | ~4.6 | 6.2 | 6.1 | 8.6 | 5.4 | 7.9 | 316 |
| DKFZ- 772 | 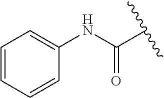 [SMILES: *C(=O)Nc1ccccc1] | A4 | 4.7 | 4.5 | <4.0 | 6.1 | 5.6 | 7.5 | 5.5 | 6.5 | 11 |
| DKFZ- 773 | 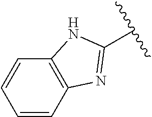 [SMILES: *c1nc2ccccc2[nH]1] | A2 | 5.0 | 4.6 | <4.0 | 6.0 | 5.3 | 8.2 | 4.4 | 6.9 | 315 |
| DKFZ- 774 | 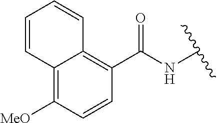 [SMILES: *NC(=O)c1ccc(OC)c2ccccc12] | A2 | 5.2 | 4.9 | <4.0 | 5.6 | 5.9 | 8.5 | ~4.9 | 7.5 | 398 |
| DKFZ- 775 | 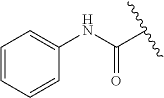 [SMILES: *C(=O)Nc1ccccc1] | A5 | 4.3 | <4.0 | <4.0 | 4.9 | 4.6 | 8.2 | <4.4 | 6.9 | >307 |
| DKFZ- 776 | 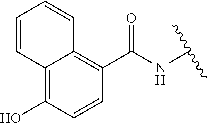 [SMILES: *NC(=O)c1ccc(O)c2ccccc12] | A2 | 5.1 | 4.8 | <4.0 | 6.2 | 5.7 | 8.3 | NM | NM | |
| DKFZ- 777 | 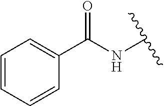 [SMILES: *NC(=O)c1ccccc1] | A6 | <4.0 | <4.0 | <4.0 | <4.0 | <4.0 | 6.4 | <4.4 | 4.5 | >1 |
| DKFZ- 805 | 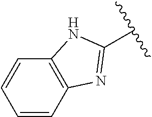 [SMILES: *c1nc2ccccc2[nH]1] | A5 | ~4.4 | 4.3 | <4.0 | 4.9 | 5.0 | 8.1 | NM | NM | |
| DKFZ- 806 | 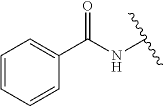 [SMILES: *NC(=O)c1ccccc1] | A5 | 4.3 | 4.1 | <4.0 | 4.6 | 4.6 | 7.7 | NM | NM | |

TABLE 2

| | | HDAC1 | HDAC6 | HDAC8 | HDAC10 | HDAC10 | |
|---|---|---|---|---|---|---|---|
| Cmpd | Structure | (ZMAL) | (ZMAL) | (FDL) | FRET | BRET | zHDAC10 |
| DH22 | 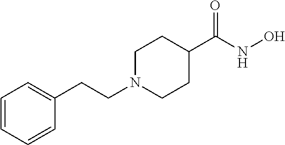 [SMILES: O=C(NO)C1CCN(CCc2ccccc2)CC1] | 10 μM (7%) 1 μM (−1%) | 10 μM (17%) 1 μM (−2%) | 10 μM (56%) 1 μM (27%) | 175 nM 260 nM | NM | 10 μM (80%) 1 μM (20%) 0.1 μM (13%) |
| DH25 | 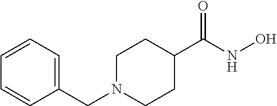 [SMILES: O=C(NO)C1CCN(Cc2ccccc2)CC1] | 10 μM (18%) 1 μM (8%) | 10 μM (46%) 1 μM (22%) | 10 μM (61%) 1 μM (36%) | 2446 nM 3433 nM | NM | 10 μM (50%) 1 μM (15%) 0.1 μM (9%) |
| DH35 | 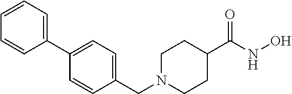 [SMILES: O=C(NO)C1CCN(Cc2ccc(-c3ccccc3)cc2)CC1] | 10 μM (35%) 1 μM (5%) | 10 μM (57%) 1 μM (11%) | 10 μM (70%) 1 μM (37%) | 1781 nM 2560 nM | NM | 10 μM (69%) 1 μM (24%) 0.1 μM (7%) |
| DH40 | 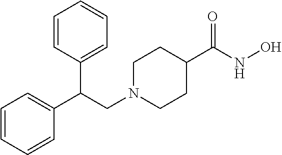 [SMILES: O=C(NO)C1CCN(CC(c2ccccc2)c2ccccc2)CC1] | 50 μM (24%) 10 μM (8%) 1 μM (−7%) | 50 μM (88%) 10 μM (65%) 1 μM (19%) | 10 μM (75%) 1 μM (42%) | 2317 nM 2968 nM | NM | 10 μM (27%) 1 μM (9%) 0.1 μM (1%) |
| DH53 | 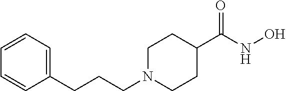 [SMILES: O=C(NO)C1CCN(CCCc2ccccc2)CC1] | 10 μM (29%) 1 μM (2%) | 10 μM (−3%) 1 μM (−23%) | 10 μM (48%) 1 μM (20%) | NM | NM | 10 μM (68%) 1 μM (19%) 0.1 μM (0%) |
| DH67 | 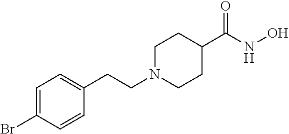 [SMILES: O=C(NO)C1CCN(CCc2ccc(Br)cc2)CC1] | 10 μM (6%) 1 μM (7%) | 10 μM (18%) 1 μM (−4%) | 10 μM (69%) 1 μM (33%) | NM | 375 nM | 10 μM (67%) 1 μM (40%) 0.1 μM (7%) |
| DH71 | 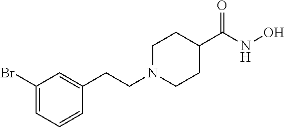 [SMILES: O=C(NO)C1CCN(CCc2cccc(Br)c2)CC1] | 10 μM (6%) 1 μM (−5%) | 10 μM (28%) 1 μM (−2%) | 10 μM (70%) 1 μM (27%) | NM | 391 nM | 10 μM (61%) 1 μM (10%) 0.1 μM (0%) |
| DH79 | 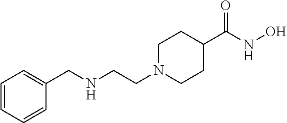 [SMILES: O=C(NO)C1CCN(CCNCc2ccccc2)CC1] | 10 μM (20%) 1 μM (6%) | 10 μM (0%) 1 μM (−15%) | 2.0 ± 0.3 μM | NM | NM | 64 ± 6 nM |
| DH88 | 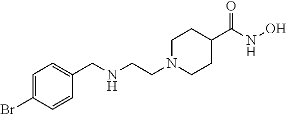 [SMILES: O=C(NO)C1CCN(CCNCc2ccc(Br)cc2)CC1] | 10 μM (10%) 1 μM (−8%) | 15 ± 4 μM | 0.7 ± 0.05 μM | NM | NM | 37 ± 4 nM |

Note:
Numbers in parenthesis indicate percent enzyme inhibition at the given concentration. The HDAC10 FRET values are IC₅₀ values are two separate measurements that were
each made in triplicate. Values that are reported with plus/minus errors are IC₅₀ values.

TABLE 3

| | | pIC₅₀ | | | | | |
|---|---|---|---|---|---|---|---|
| Com- | | HDAC1 | HDAC2 | HDAC3 | HDAC6 | HDAC8 | HDAC10 |
| pound | Structure | Glo | Glo | Glo | Glo | Glo | FRET |
| DKFZ- 825 | 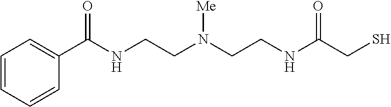 [SMILES: CN(CCNC(=O)CS)CCNC(=O)c1ccccc1] | 4.9 | 4.9 | <4.0 | 5.4 | <4.0 | 7.9 |

### REFERENCES

(1) Taunton, J., et al. A mammalian histone deacetylase related to the yeast transcriptional regulator Rpd3p. Science 1996, 272, 408-411.
(2) Marks, P. A.; Breslow, R. Dimethyl sulfoxide to vorinostat: development of this histone deacetylase inhibitor as an anticancer drug. Nat. Biotechnol. 2007, 25, 84-90.
(3) Falkenberg, K. J.; Johnstone, R. W. Histone deacetylases and their inhibitors in cancer, neurological diseases and immune disorders. Nat. Rev. Drug Disc. 2014, 13, 673-691.
(4) Roche, J.; Bertrand, P. Inside HDACs with more selective HDAC inhibitors. Eur. J. Med. Chem. 2016, 121, 451-483.
(5) Kutil, Z., et al. Histone deacetylase 11 is a fatty-acid deacylase. ACS Chem. Biol. 2018, 13, 685-693.
(6) Hai, Y., et al. Histone deacetylase 10 structure and molecular function as a polyamine deacetylase. Nat. Commun. 2017, 8, 15368.
(7) Aramsangtienchai, P., et al. HDAC8 catalyzes the hydrolysis of long chain fatty acyl lysine. ACS Chem. Biol. 2016, 11, 2685-2692.
(8) Feldman, J. L., et al. Activation of the protein deacetylase SIRT6 by long-chain fatty acids and widespread deacylation by mammalian sirtuins. J. Biol. Chem. 2013, 288, 31350-31356.
(9) Moreno-Yruela, C., et al. Histone Deacetylase 11 Is an ε-N-Myristoyllysine Hydrolase. Cell Chem. Biol. 2018, 25, 849-856.
(10) Witt, O., et al. HDAC family: What are the cancer relevant targets? Cancer Lett. 2009, 277, 8-21.
(11) Balasubramanian, S., et al. Isoform-specific histone deacetylase inhibitors: The next step? Cancer Lett. 2009, 280, 211-221.
(12) Bradner, J. E., et al. Chemical phylogenetics of histone deacetylases. Nat. Chem. Biol. 2010, 6, 238-243.
(13) Boskovic, Z. V., et al. Inhibition of zinc-dependent histone deacetylases with a chemically triggered electrophile. ACS Chem. Biol. 2016, 11, 1844-1851.
(14) Lobera, M., et al. Selective class IIa histone deacetylase inhibition via a nonchelating zinc-binding group. Nat. Chem. Biol. 2013, 9, 319-325.
(15) Mai, A., et al. Class II (IIa)-selective histone deacetylase inhibitors. 1. Synthesis and biological evaluation of novel (aryloxopropenyl)pyrrolyl hydroxyamides. J. Med. Chem. 2005, 48, 3344-3353.
(16) Martin, M. W., et al. Discovery of novel N-hydroxy-2-arylisoindoline-4-carboxamides as potent and selective inhibitors of HDAC11. Bioorg. Med. Chem. Lett. 2018, 28, 2143-2147.
(17) Marek, L., et al. Histone deacetylase (HDAC) inhibitors with a novel connecting unit linker region reveal a selectivity profile for HDAC4 and HDAC5 with improved activity against chemoresistant cancer cells. J. Med. Chem. 2013, 56, 427-436.
(18) Haggarty, S. J., et al. Domain-selective small-molecule inhibitor of histone deacetylase 6 (HDAC6)-mediated tubulin deacetylation. Proc. Nat. Acad. Sci. 2003, 100, 4389-4394.
(19) Butler, K. V., et al. Rational design and simple chemistry yield a superior, neuroprotective HDAC6 inhibitor, tubastatin A. J. Am. Chem. Soc. 2010, 132, 10842-10846.
(20) Jochems, J., et al. Antidepressant-like properties of novel HDAC6-selective inhibitors with improved brain bioavailability. Neuropsychopharmacology 2013, 39, 389-400.
(21) Arrowsmith, C. H., et al. The promise and peril of chemical probes. Nat. Chem. Bio. 2015, 11, 536-541.
(22) Fischer, D. D., et al. Isolation and characterization of a novel class II histone deacetylase, HDAC10. J. Biol. Chem. 2002, 277, 6656-6666.
(23) Guardiola, A. R.; Yao, T. P. Molecular cloning and characterization of a novel histone deacetylase HDAC10. J. Biol. Chem. 2002, 277, 3350-3356.
(24) Kao, H. Y., et al. Isolation and characterization of mammalian HDAC10, a novel histone deacetylase. J. Biol. Chem. 2002, 277, 187-193.
(25) Lai, I. L., et al. Histone deacetylase 10 relieves repression on the melanogenic program by maintaining the deacetylation status of repressors. J. Biol. Chem. 2010, 285, 7187-7196.
(26) Park, B. L., et al. HDAC10 promoter polymorphism associated with development of HCC among chronic HBV patients. Biochem. Biophys. Res. Commun. 2007, 363, 776-781
(27) Kotian, S., et al. Histone deacetylases 9 and 10 are required for homologous recombination. J. Biol. Chem. 2011, 286, 7722-7726.
(28) Park, J. H., et al. Class II histone deacetylases play pivotal roles in heat shock protein 90-mediated proteasomal degradation of vascular endothelial growth factor receptors. Biochem. Biophys. Res. Commun. 2008, 368, 318-322.
(29) Jin, Z., et al. Decreased expression of histone deacetylase 10 predicts poor prognosis of gastric cancer patients. Int. J. Clin. Exp. Pathol. 2014, 7, 5872-5879.
(30) Osada, H., et al. Reduced expression of class II histone deacetylase genes is associated with poor prognosis in lung cancer patients. International journal of cancer 2004, 112, 26-32.
(31) Powers, J., et al. Expression and function of histone deacetylase 10 (HDAC10) in B cell malignancies. Methods in molecular biology 2016, 1436, 129-145.
(32) Song, C., et al. Histone deacetylase (HDAC) 10 suppresses cervical cancer metastasis through inhibition of matrix metalloproteinase (MMP) 2 and 9 expression. J. Biol. Chem. 2013, 288, 28021-28033.
(33) Fan, W., et al. Histone deacetylase 10 suppresses proliferation and invasion by inhibiting the phosphorylation of beta-catenin and serves as an independent prognostic factor for human clear cell renal cell carcinoma. Int. J. Clin. Exp. Med. 2015, 8, 3734-3742.
(34) Islam, M. M., et al. HDAC10 as a potential therapeutic target in ovarian cancer. Gynecol. Oncol. 2017, 144, 613-620.
(35) Oehme, I., et al. Histone deacetylase 10-promoted autophagy as a druggable point of interference to improve the treatment response of advanced neuroblastomas. Autophagy 2013, 9, 2163-2165.
(36) Yang, Y., et al. HDAC10 promotes lung cancer proliferation via AKT phosphorylation. Oncotarget 2016, 7, 59388-59401.
(37) Oehme, I., et al. Histone deacetylase 10 promotes autophagy-mediated cell survival. Proc. Nat. Acad. Sci. 2013, 110, E2592-E2601.
(38) Ridinger, J., et al. Dual role of HDAC10 in lysosomal exocytosis and DNA repair promotes neuroblastoma chemoresistance. Sci. Rep. 2018, 8, 10039.
(39) Bantscheff, M., et al. Chemoproteomics profiling of HDAC inhibitors reveals selective targeting of HDAC complexes. Nat. Biotechnol. 2011, 29, 255-265.
(40) Kolbinger, F. R., et al. The HDAC6/8/10 inhibitor TH34 induces DNA damage-mediated cell death in human high-grade neuroblastoma cell lines. Arch. Toxicol. 2018, 92, 2649-2664.
(41) Géraldy, M., et al. Selective inhibition of histone deacetylase 10: Hydrogen bonding to the gatekeeper residue is implicated. J. Med. Chem. 2019, 62, 4426-4443
(42) Wang, M., et al. A Novel Role for Histone Deacetylase 10 (HDAC10) in the Regulation of PD-L1 Expression and Immune Tolerance Mediated By Antigen Presenting Cells (APCs). Blood 2017 130:3561.
(43) Rajak, H., et al. Appraisal of GABA and PABA as linker: Design and synthesis of novel benzamide based histone deacetylase inhibitors. Eur. J. Med. Chem. 2012, 53, 390-397.
(44) Herbst-Gervasoni, C. and Christianson, D. W. Binding of N⁸-Acetylspermidine Analogues to Histone Deacetylase 10 Reveals Molecular Strategies for Blocking Polyamine Deacetylation. Biochemistry 2019, 58, 4957-4969.
(45) Marek, M.; Kannan, S.; Hauser, A. T.; Moraes Mourao, M.; Caby, S.; Cura, V.; Stolfa, D. A.; Schmidtkunz, K.; Lancelot, J.; Andrade, L.; Renaud, J. P.; Oliveira, G.; Sippl, W.; Jung, M.; Cavarelli, J.; Pierce, R. J.; Romier, C., Structural basis for the inhibition of histone deacetylase 8 (HDAC8), a key epigenetic player in the blood fluke Schistosoma mansoni. PLoS Pathog. 2013, 9, e1003645.
(46) Dahiya, S. et al. HDAC10 deletion promotes Foxp3+ T-regulatory cell function. Sci. Rep. 2020, 10, 424.
(47) Kozikowski, A. P.; Chen, Y.; Gaysin, A.; Chen, B.; D'Annibale, M. A.; Suto, C. M.; Langley, B. C. Functional differences in epigenetic modulators-superiority of mercaptoacetamide-based histone deacetylase inhibitors relative to hydroxamates in cortical neuron neuroprotection studies. J. Med. Chem. 2007, 50, 3054-3061. DOI: 10.1021/jm070178x.
(48) Kang, S. O.; Powell, D.; Day, V. W.; Bowman-James, K. Trapped bifluoride. Angew. Chem. Int. Ed. Engl. 2006, 45, 1921-1925. DOI: 10.1002/anie.200504066.
(49) O'Meara, J. A.; Yoakim, C.; Bonneau, P. R.; Bös, M.; Cordingley, M. G.; Déziel, R.; Doyon, L.; Duan, J.; Garneau, M.; Guse, I.; Landry, S.; Malenfant, E.; Naud, J.; Ogilvie, W. W.; Thavonekham, B.; Simoneau, B. Novel 8-substituted dipyridodiazepinone inhibitors with a broad-spectrum of activity against HIV-1 strains resistant to non-nucleoside reverse transcriptase inhibitors. J. Med. Chem. 2005, 48, 5580-5588. DOI:
10.1021/jm050255t.

1. An HDAC10 inhibitor of Formula (I)
CAP-(CR^{y*}R^{y*′})ₙ—NR²—CR³R^{3′}—CR⁴R^{4′}—CR⁵R^{5′}—ZBD (I)
wherein:
CAP is a capping group selected from the groups of aryl-X— and heteroaryl-X—, wherein X is absent or is selected from —C(═O)—NR¹—, —NR—C(═O)—, —S(═O)₂—NR¹—, —NR—S(═O)₂—, —S(═O)(═NR)—NR′—, —NR—S(═O)(═NR)—, —C(═NR)—, —O—, —S—, —S(═O)—, —S(═O)₂—, and —C(═O)—,
wherein
R and R¹ are each independently a residue selected from H and a substituent selected from linear or branched C₁₋₄-alkyl, cyclopropyl, benzyl, aryl and heteroaryl, wherein said substituent is optionally further substituted, in particular by a further substituent selected from the list of —F, —OH, —OR, and —NR₂;
n is an integer selected from 1, 2 and 3;
y is an integer taking the values from the range of 1 to n;
ZBD is a zinc-binding domain selected from the group of:
—C(═O)—NH—OH, —C(═S)—NH—OH, —C(═N—OH)—NHOH, —C(═O)NH—R⁶, —C(═N—OH)—C(═O)NH—R⁶, —C(═O)CF₃, —C(═O)CH₂SH, C(═S)CH₂SH, —SH, —C(═NH)—NH—OH, and —C(═N—OH)—NH₂, wherein R⁶ is a residue selected from H, linear or branched C₁₋₄-alkyl, -cyclopropyl, and benzyl;
and
R² is a residue selected from H and a substituent selected from linear or branched C₁₋₄-alkyl, cyclopropyl, benzyl, aryl and heteroaryl, wherein said substituent is optionally further substituted, in particular by a further substituent selected from the list of —F, —OH, —OR, and —NR₂, and each R^{y*}, each R^{y*′}, R³, R^{3′}, R⁴, R^{4′}, R⁵, and R^{5′} are independently selected from residues H, CH₃, F, CFH₂, CF₂H and CF₃, provided that in total not more than three of said residues are different from —H;
or
two residues selected from the R¹, R^{y*}, R^{y*′} and R² residues, together with the atoms they are attached to, form a three to six-membered ring, wherein said three to six-membered ring is optionally further substituted, in particular by a further substituent selected from the list of —F, —OH, —OR, and —NR₂, and the remaining residues R¹, each R^{y*}, each R^{y*′}, R³, R^{3′}, R⁴, R^{4′}, R⁵, and R^{5′} are independently selected from residues H, CH₃, F, CFH₂, CF₂H and CF₃, provided that in total not more than three of said residues are different from —H;
or
two residues selected from the R², R³, R^{3′}, R⁴, R^{4′}, R⁵, and R^{5″} residues, together with the atoms they are attached to, form a three to six-membered ring, and the remaining residues R¹, each R^{y*}, each R^{y*′}, R³, R^{3′}, R⁴, R^{4′}, R⁵, and R^{5′} are independently selected from residues H, CH₃, F, CFH₂, CF₂H and CF₃, provided that in total not more than three of said residues are different from —H.

2. An HDAC10 inhibitor of Formula (Ia)
CAP-(CR^{y*}R^{y*′})ₙ—NR²—CR³R^{3′}—CR⁴R^{4′}—CR⁵R^{5′}—ZBD (Ia)
wherein:
CAP is a capping group selected from the groups of aryl-X— and heteroaryl-X—, wherein X is —CH₂—NR¹—,
and wherein the other residues are as defined in claim 1.

3. The HDAC10 inhibitor of claim 1 or 2, wherein ZBD is —C(═O)—NH—OH.

4. An HDAC10 inhibitor of Formula (Ib)
CAP-(CR^{y*}R^{y*′})ₙ—NR²—CR³R^{3′}—CR⁴R^{4′}—NR⁵—ZBD (Ib)
wherein:
CAP is a capping group selected from the groups of aryl-X— and heteroaryl-X—, wherein X is absent or is selected from —C(═O)—NR¹—, —NR—C(═O)—, —S(═O)₂—NR¹—, —NR—S(═O)₂—, —S(═O)(═NR)—NR′—, —NR—S(═O)(═NR)—, —C(═NR)—, —O—, —S—, —S(═O)—, —S(═O)₂—, —C(═O)—, and —CH₂—NR¹—,
wherein
R and R¹ are each independently a residue selected from H and a substituent selected from linear or branched C₁₋₄-alkyl, cyclopropyl, benzyl, aryl and heteroaryl, wherein said substituent is optionally further substituted, in particular by a further substituent selected from the list of —F, —OH, —OR, and —NR₂;
n is an integer selected from 1, 2 and 3;
y is an integer taking the values from the range of 1 to n;
ZBD is a zinc-binding domain selected from the group of:
—C(═O)—NH—OH, —C(═S)—NH—OH, —C(═N—OH)—NHOH, —C(═O)NH—R⁶, —C(═N—OH)—C(═O)NH—R⁶, —C(═O)CF₃, —C(═O)CH₂SH, C(═S)CH₂SH, —C(═NH)—NH—OH, and —C(═N—OH)—NH₂, wherein R⁶ is a residue selected from H, linear or branched C₁₋₄-alkyl, -cyclopropyl, and benzyl, and
and
R² and R⁵ are each a residue independently selected from H and a substituent selected from linear or branched C₁₋₄-alkyl, cyclopropyl, benzyl, aryl and heteroaryl, wherein said substituent is optionally further substituted, in particular by a further substituent selected from the list of —F, —OH, —OR, and —NR₂, and each R^{y*}, each R^{y*′}, R³, R^{3′}, R⁴, and R^{4′} are independently selected from residues H, CH₃, F, CFH₂, CF₂H and CF₃, provided that in total not more than three of said residues are different from —H;
or
two residues selected from the R¹, R^{y*}, R^{y*′} and R² residues, together with the atoms they are attached to, form a three to six-membered ring, wherein said three to six-membered ring is optionally further substituted, in particular by a further substituent selected from the list of —F, —OH, —OR, and —NR₂, and the remaining residues R¹, each R^{y*}, each R^{y*}, R³, R^{3′}, R⁴, and R^{4′} are independently selected from residues H, CH₃, F, CFH₂, CF₂H and CF₃, provided that in total not more than three of said residues are different from —H;
or
two residues selected from the R², R³, R^{3′}, R⁴, R^{4′}, and R⁵ residues, together with the atoms they are attached to, form a three to six-membered ring, and the remaining residues R¹, each R^{y*}, each R^{y*′}, R³, R^{3′}, R⁴, R^{4′}, and R⁵ are independently selected from residues H, CH₃, F, CFH₂, CF₂H and CF₃, provided that in total not more than three of said residues are different from —H.

5. The HDAC10 inhibitor of claim 4, wherein ZBD is —C(═O)CH₂SH.

6. The HDAC10 inhibitor of any one of claims 1 to 5, wherein CAP is a capping group selected from the group of: aryl-C(═O)—NH—, heteroaryl-C(═O)—NH—, and aryl-NH—C(═O)—, and heteroaryl-NH—C(═O)—.

7. The HDAC10 inhibitor of claim 6, wherein CAP is a capping group selected from the group of phenyl-NH—C(═O)—, phenyl-C(═O)—NH—, 1-naphthyl-C(═O)—NH—, and 7-indazolyl-C(═O)—NH—.

8. The HDAC10 inhibitor of claim 7, wherein CAP is phenyl-NH—C(═O)— or phenyl-C(═O)—NH—.

9. The HDAC10 inhibitor of claim 8, wherein CAP is phenyl-NH—C(═O)—.

10. The HDAC10 inhibitor of claim 8, wherein CAP is phenyl-C(═O)—NH—.

11. The HDAC10 inhibitor of any one of claims 1 to 5, wherein CAP is benzimidazol-2-yl.

12. The HDAC10 inhibitor of any one of claims 1 to 12, wherein n is 2.

13. The HDAC10 inhibitor of any one of claims 1 to 12, wherein n is 3.

14. The HDAC10 inhibitor of any one of claims 1 to 13, wherein R² is a residue selected from H, methyl, ethyl, n-propyl, i-propyl, n-butyl, cyclopropyl, and benzyl.

15. The HDAC10 inhibitor of claim 14, wherein R² is methyl,

16. The HDAC10 inhibitor of any one of claims 1 to 15, wherein each R^{y*}, each R^{y*′}, R³, R^{3′}, R⁴, R^{4′}, R⁵, and R^{5′} are each —H.

17. The HDAC10 inhibitor of any one of claims 1 to 16, wherein the HDAC10 inhibitor is selected from the group of DKFZ-711, DKFZ-775, DKFZ-772, DKFZ-728, DKFZ-777, DKFZ-773, DKFZ-748, DKFZ-750, DKFZ-757, DKFZ-771, DKFZ-774, DKFZ-746, DKFZ-747, DKFZ-749, DKFZ-751, DKFZ-752, DKFZ-753, DKFZ-754, DKFZ-755, DKFZ-756, DKFZ-769, DKFZ-776, DKFZ-758, DKFZ-759, DKFZ-767, DKFZ-770, DKFZ-714, DKFZ-715, DKFZ-716, DKFZ-717, DKFZ-718, DKFZ-724, DH22, DH25, DH35. DH40, DH53, DH67, DH71, DH79, DH88, and DKFZ-825

18. The HDAC10 inhibitor of claim 17, wherein the HDAC10 inhibitor is selected from the group of DKFZ-711, DKFZ-714, DKFZ-715, DKFZ-716, DKFZ-717, DKFZ-718, DKFZ-724 and DKFZ-728.

19. A pharmaceutically acceptable salt form of the HDAC10 inhibitor of any one of claims 1 to 18.

20. The pharmaceutically acceptable salt form of claim 19, wherein the HDAC10 of any one of claims 1 to 18 is reacted with an acid selected from the group of: hydrochloric, hydrobromic, sulfuric, sulfamic, phosphoric, nitric; acetic, propionic, succinic, glycolic, stearic, lactic, malic, tartaric, citric, ascorbic, palmoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic acid, in particular hydrochloric acid.

21. A pharmaceutical composition comprising an HDAC10 inhibitor of any one of claims 1 to 18, or a pharmaceutically acceptable salt form of an HDAC10 inhibitor of claim 19 or 20.

22. An HDAC10 inhibitor of any one of claims 1 to 18, a pharmaceutically acceptable salt form of an HDAC10 inhibitor of claim 19 or 20, or the pharmaceutical composition of claim 21 for use in the treatment of a disease selected from the list of cancer, autoimmune disorders and neurodegeneration.

23. An HDAC10 inhibitor of any one of claims 1 to 18, a pharmaceutically acceptable salt form of an HDAC10 inhibitor of claim 19 or 20, or a pharmaceutical composition of claim 21 for the use of claim 22, wherein autophagy is upregulated in the cells of said disease.
